(19) United States
(12) Patent Application Publication
(10) Pub. No.: US 2011/0207701 A1
(43) Pub. Date: Aug. 25, 2011

(54) BORON-CONTAINING SMALL MOLECULES AS ANTIPROTOZOAL AGENTS

(75) Inventors: Huchen Zhou, Shanghai (CN); Dazhong Ding, Hanzhong (CN); Yasheen Zhou, Moraga, CA (US); Yong-Kang Zhang, San Jose, CA (US); Jacob J. Plattner, Orinda, CA (US)

(73) Assignee: Anacor Pharmaceuticals, Inc., Palo Alto, CA (US)

(21) Appl. No.: 12/857,305

(22) Filed: Aug. 16, 2010

Related U.S. Application Data
(60) Provisional application No. 61/235,296, filed on Aug. 19, 2009.

(51) Int. Cl. A61K 31/69; C07F 5/04; A01N 55/08; A61P 33/02; A61P 33/06; A01P 1/00

(52) U.S. Cl. 514/64; 558/288; 548/110; 544/229; 544/69; 546/13; 548/405

(57) ABSTRACT

This invention provides, among other things, novel compounds useful for treating protozoal infections, pharmaceutical compositions containing such compounds, as well as combinations of these compounds with at least one additional therapeutically effective agent.

### CROSS-REFERENCE TO RELATED APPLICATIONS

This application claims the benefit of U.S. Provisional Pat. App. No. 61/235,296, filed Aug. 19, 2009, which is incorporated by reference in its entirety for all purposes.

### BACKGROUND OF THE INVENTION

The global rise of protozoa resistant to antimicrobials in general, poses a major threat. Deployment of massive quantities of antimicrobial agents into the ecosphere during the past 60 years has introduced a powerful selective pressure for the emergence and spread of antimicrobial-resistant pathogens. Thus, there is a need to discover new broad spectrum antimicrobials, such as antiprotozoals, useful in combating microorganisms, especially those with multidrug-resistance.

Boron-containing molecules, such as oxaboroles, useful as antimicrobials have been described previously, such as in U.S. Pat. Pubs. US20060234981 and US20070155699. Generally speaking, an oxaborole has the following structure and substituent numbering system:

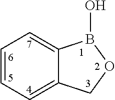

It has now been discovered that certain classes of oxaboroles which are surprisingly effective antiprotozoals. This, and other uses of these oxaboroles are described herein.

### SUMMARY OF THE INVENTION

This invention provides, among other things, novel compounds useful for treating protozoa infections, pharmaceutical compositions containing such compounds, as well as combinations of these compounds with at least one additional therapeutically effective agent.

### BRIEF DESCRIPTION OF THE DRAWINGS

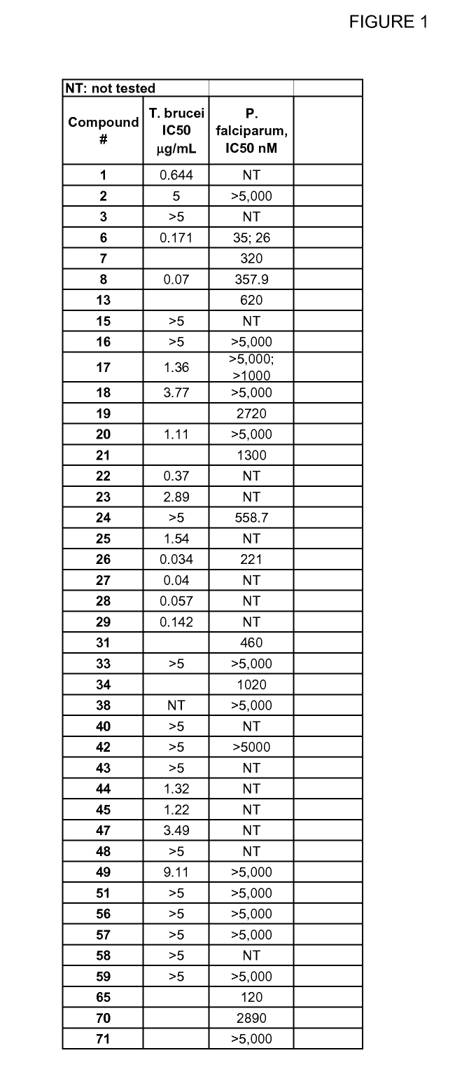

Biological data for exemplary compounds of the invention is provided in FIG. 1.

### DETAILED DESCRIPTION OF THE INVENTION

### I. Definitions and Abbreviations

As used herein, the singular forms “a,” “an”, and “the” include plural references unless the context clearly dictates otherwise. For example, reference to “an active agent” includes a single active agent as well as two or more different active agents in combination. It is to be understood that present teaching is not limited to the specific dosage forms, carriers, or the like, disclosed herein and as such may vary.

The abbreviations used herein generally have their conventional meaning within the chemical and biological arts.

The following abbreviations have been used: Ac is acetyl; AcOH is acetic acid; ACTBr is cetyltrimethylammonium bromide; AIBN is azobisisobutyronitrile or 2,2 azobisisobutyronitrile; aq. is aqueous; Ar is aryl; B₂pin₂ is bis(pinacolato)diboron; Bn is, in general, benzyl [see Cbz for one example of an exception]; (BnS)₂ is benzyl disulfide; BnSH is benzyl thiol or benzyl mercaptan; BnBr is benzyl bromide; Boc is tert-butoxy carbonyl; Boc₂O is di-tert-butyl dicarbonate; Bz is, in general, benzoyl; BzOOH is benzoyl peroxide; Cbz or Z is benzyloxycarbonyl or carboxybenzyl; Cs₂CO₃ is cesium carbonate; CSA is camphor sulfonic acid; CTAB is cetyltrimethylammonium bromide; Cy is cyclohexyl; DABCO is 1,4-diazabicyclo[2.2.2]octane; DCM is dichloromethane or methylene chloride; DHP is dihydropyran; DIAD is diisopropyl azodicarboxylate; DIEA or DIPEA is N,N-diisopropylethylamine; DMAP is 4-(dimethylamino)pyridine; DME is 1,2-dimethoxyethane; DMF is N,N-dimethylformamide; DMSO is dimethylsulfoxide; equiv or eq. is equivalent; EtOAc is ethyl acetate; EtOH is ethanol; Et₂O is diethyl ether; EDCI is N-(3-dimethylaminopropyl)-N′-ethylcarbodiimide hydrochloride; ELS is evaporative light scattering; equiv or eq is equivalent; h is hours; HATU is O-(7-azabenzotriazol-1-yl)-N,N,N′,N′-tetramethyluronium hexafluorophosphate; HOBt is N-hydroxybenzotriazole; HCl is hydrochloric acid; HPLC is high pressure liquid chromatography; ISCO Companion is automated flash chromatography equipment with fraction analysis by UV absorption available from Presearch; KOAc or AcOK is potassium acetate; K₂CO₃ is potassium carbonate; LiAlH₄ or LAH is lithium aluminum hydride; LDA is lithium diisopropylamide; LHMDS is lithium bis(trimethylsilyl) amide; KHMDS is potassium bis(trimethylsilyl) amide; LiOH is lithium hydroxide; m-CPBA is 3-chloroperoxybenzoic acid; MeCN or ACN is methyl cyanide or cyanomethane or ethanenitrile or acetonitrile which are all names for the same compound; MeOH is methanol; MgSO₄ is magnesium sulfate; mins or min is minutes; Mp or MP is melting point; NaCNBH₃ is sodium cyanoborohydride; NaOH is sodium hydroxide; Na₂SO₄ is sodium sulfate; NBS is N-bromosuccinimide; NH₄Cl is ammonium chloride; NIS is N-iodosuccinimide; N₂ is nitrogen; NMM is N-methylmorpholine; n-BuLi is n-butyllithium; overnight is O/N; PdCl₂(pddf) is 1,1′-Bis(diphenylphosphino) ferrocene]dichloropalladium(II); Pd/C is the catalyst known as palladium on carbon; Pd₂(dba)₃ is an organometallic catalyst known as tris(dibenzylideneacetone) dipalladium(0); Ra Ni or Raney Ni is Raney nickel; Ph is phenyl; PMB is p-methoxybenzyl; PrOH is 1-propanol; iPrOH is 2-propanol; POCl₃ is phosphorus chloride oxide; PTSA is para-toluene sulfonic acid; Pyr. or Pyr or Py as used herein means pyridine; RT or rt or r.t. is room temperature; sat. is saturated; Si-amine or Si—NH₂ is amino-functionalized silica, available from SiliCycle; Si-pyr is pyridyl-functionalized silica, available from SiliCycle; TEA or Et₃N is triethylamine; TFA is trifluoroacetic acid; Tf₂O is trifluoromethanesulfonic anhydride; THF is tetrahydrofuran; TFAA is trifluoroacetic anhydride; THP is tetrahydropyranyl; TMSI is trimethylsilyl iodide; H₂O is water; diNO₂PhSO₂Cl is dinitrophenyl sulfonyl chloride; 3-F-4-NO₂-PhSO₂Cl is 3-fluoro-4-nitrophenylsulfonyl chloride; 2-MeO-4-NO₂-PhSO₂Cl is 2-methoxy-4-nitrophenylsulfonyl chloride; and (EtO)₂POCH₂COOEt is a triethylester of phosphonoacetic acid known as triethyl phosphonoacetate.

“Compound of the invention,” as used herein refers to the compounds discussed herein, salts (e.g. pharmaceutically acceptable salts), prodrugs, solvates and hydrates of these compounds.

“Combination of the invention,” as used herein refers to the compounds and antiprotozoals discussed herein as well as acids, bases, salt forms (such as pharmaceutically acceptable salts), prodrugs, solvates and hydrates of these compounds and antiprotozoals.

“Boron containing compounds”, as used herein, refers to the compounds of the invention that contain boron as part of their chemical formula.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “poly” as used herein means at least 2. For example, a polyvalent metal ion is a metal ion having a valency of at least 2.

“Moiety” refers to a radical of a molecule that is attached to the remainder of the molecule.

The symbol [symbol] , whether utilized as a bond or displayed perpendicular to a bond, indicates the point at which the displayed moiety is attached to the remainder of the molecule.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). In some embodiments, the term “alkyl” means a straight or branched chain, or combinations thereof, which may be fully saturated, mono- or polyunsaturated and can include di- and multivalent radicals. Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the invention. A “lower alkyl” or “lower alkylene” is a shorter chain alkyl or alkylene group, generally having eight or fewer carbon atoms.

The term “alkenylene” by itself or as part of another substituent means a divalent radical derived from an alkene.

The term “cycloalkylene” by itself or as part of another substituent means a divalent radical derived from a cycloalkane.

The term “heteroalkylene” by itself or as part of another substituent means a divalent radical derived from an heteroalkane.

The term “heterocycloalkylene” by itself or as part of another substituent means a divalent radical derived from an heterocycloalkane.

The term “arylene” by itself or as part of another substituent means a divalent radical derived from an aryl.

The term “heteroarylene” by itself or as part of another substituent means a divalent radical derived from heteroaryl.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom. In some embodiments, the term “heteroalkyl,” by itself or in combination with another term, means a stable straight or branched chain, or combinations thereof, consisting of the stated number of carbon atoms and at least one heteroatom. In an exemplary embodiment, the heteroatoms can be selected from the group consisting of B, O, N and S, and wherein the nitrogen and sulfur atoms may optionally be oxidized and the nitrogen heteroatom may optionally be quaternized. The heteroatom(s) B, O, N and S may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —CH₂—CH═N—OCH₃, and —CH═CH—N(CH₃)—CH₃. Up to two heteroatoms may be consecutive, such as, for example, —CH₂—NH—OCH₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′ C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 or 2 or 3 rings), which are fused together or linked covalently. The term “heteroaryl” refers to aryl groups (or rings) that contain from one to four heteroatoms. In an exemplary embodiment, the heteroatom is selected from B, N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —R′, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR′″″-C(NR′R″R′″)═NR″″, —NR″″—C(NR′R″)═NR′″, —S(O)R′, —S(O)₂R′, —S(O)₂NR′R″, —NR″SO₂R′, —CN, —NO₂, —N₃, —CH(Ph)₂, fluoro(C₁-C₄)alkoxy, and fluoro(C₁-C₄)alkyl, in a number ranging from zero to (2 m′+1), where m′ is the total number of carbon atoms in such radical. R′, R″, R′″, R″″ and R′″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″, R″″ and R′″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: —R′, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR′″″-C(NR′R″R′″)═NR″″, —NR″″-C(NR′R″)═NR′″, —S(O)R′, —S(O)₂R′, —S(O)₂NR′R″, —NR″SO₂R′, —CN, —NO₂, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, R″″ and R′″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″, R″″ and R′″″ groups when more than one of these groups is present.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted C₁ or C₂ or C₃ or C₄ or C₅ or C₆ alkyl.

“Ring” as used herein, means a substituted or unsubstituted cycloalkyl, substituted or unsubstituted heterocycloalkyl, substituted or unsubstituted aryl, or substituted or unsubstituted heteroaryl. A ring includes fused ring moieties. The number of atoms in a ring is typically defined by the number of members in the ring. For example, a “5- to 7-membered ring” means there are 5 or 6 or 7 atoms in the encircling arrangement. Unless otherwise specified, the ring optionally includes a heteroatom. Thus, the term “5 to 7-membered ring” or “5 or 6 or 7 membered ring” includes, for example phenyl, pyridinyl and piperidinyl. The term “5 to 7-membered heterocycloalkyl ring” “5 or 6 or 7-membered heterocycloalkyl ring”, on the other hand, would include pyridinyl and piperidinyl, but not phenyl. The term “ring” further includes a ring system comprising more than one “ring”, wherein each “ring” is independently defined as above.

As used herein, the term “heteroatom” includes atoms other than carbon (C) and hydrogen (H). Examples include oxygen (O), nitrogen (N) sulfur (S), silicon (Si), germanium (Ge), aluminum (Al) and boron (B).

The term “leaving group” means a functional group or atom which can be displaced by another functional group or atom in a substitution reaction, such as a nucleophilic substitution reaction. By way of example, representative leaving groups include triflate, chloro, bromo and iodo groups; sulfonic ester groups, such as mesylate, tosylate, brosylate, nosylate and the like; and acyloxy groups, such as acetoxy, trifluoroacetoxy and the like.

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, substituted or unsubstituted cycloalkyl and substituted or unsubstituted heterocycloalkyl groups.

By “effective” amount of a drug, formulation, or permeant is meant a sufficient amount of an active agent to provide the desired local or systemic effect. A “Topically effective,” “pharmaceutically effective,” or “therapeutically effective” amount refers to the amount of drug needed to effect the desired therapeutic result.

“Topically effective” refers to a material that, when applied to the skin, nail, hair, claw or hoof produces a desired pharmacological result either locally at the place of application or systemically as a result of transdermal passage of an active ingredient in the material.

The term “pharmaceutically acceptable salt” is meant to include a salt of a compound of the invention which is prepared with relatively nontoxic acids or bases, depending on the particular substituents found on the compounds described herein. When compounds of the invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino (such as choline or diethylamine or amino acids such as d-arginine, 1-arginine, d-lysine, or 1-lysine), or magnesium salt, or a similar salt. When compounds of the invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science 66: 1-19 (1977)). Certain specific compounds of the invention contain both basic and acidic functionalities that allow the compounds to be converted into either base or acid addition salts.

The neutral forms of the compounds are preferably regenerated by contacting the salt with a base or acid and isolating the parent compounds in the conventional manner. The parent form of the compound differs from the various salt forms in certain physical properties, such as solubility in polar solvents.

In addition to salt forms, the invention provides compounds which are in a prodrug form. Prodrugs of the compounds described herein readily undergo chemical changes under physiological conditions to provide the compounds of the invention. Additionally, prodrugs can be converted to the compounds of the invention by chemical or biochemical methods in an ex vivo environment.

Certain compounds of the invention can exist in unsolvated forms as well as solvated forms, including hydrated forms. In general, the solvated forms are equivalent to unsolvated forms and are encompassed within the scope of the invention. Certain compounds of the invention may exist in multiple crystalline or amorphous forms.

Certain compounds of the invention possess asymmetric carbon atoms (optical centers) or double bonds; the racemates, diastereomers, geometric isomers and individual isomers are encompassed within the scope of the invention. The graphic representations of racemic, ambiscalemic and scalemic or enantiomerically pure compounds used herein are taken from Maehr, J. Chem. Ed. 1985, 62: 114-120. Solid and broken wedges are used to denote the absolute configuration of a stereocenter unless otherwise noted. When the compounds described herein contain olefinic double bonds or other centers of geometric asymmetry, and unless specified otherwise, it is intended that the compounds include both E and Z geometric isomers. Likewise, all tautomeric forms are included.

Compounds of the invention can exist in particular geometric or stereoisomeric forms. The invention contemplates all such compounds, including cis- and trans-isomers, (−)- and (+)-enantiomers, (R)- and (S)-enantiomers, diastereomers, (D)-isomers, (L)-isomers, the racemic mixtures thereof, and other mixtures thereof, such as enantiomerically or diastereomerically enriched mixtures, as falling within the scope of the invention. Additional asymmetric carbon atoms can be present in a substituent such as an alkyl group. All such isomers, as well as mixtures thereof, are intended to be included in this invention.

Optically active (R)- and (S)-isomers and d and/isomers can be prepared using chiral synthons or chiral reagents, or resolved using conventional techniques. If, for instance, a particular enantiomer of a compound of the invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

The compounds of the invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the invention, whether radioactive or not, are intended to be encompassed within the scope of the invention.

The term “pharmaceutically acceptable carrier” or “pharmaceutically acceptable vehicle” refers to any formulation or carrier medium that provides the appropriate delivery of an effective amount of an active agent as defined herein, does not interfere with the effectiveness of the biological activity of the active agent, and that is sufficiently non-toxic to the host or patient. Representative carriers include water, oils, both vegetable and mineral, cream bases, lotion bases, ointment bases and the like. These bases include suspending agents, thickeners, penetration enhancers, and the like. Their formulation is well known to those in the art of cosmetics and topical pharmaceuticals. Additional information concerning carriers can be found in Remington: The Science and Practice of Pharmacy, 21st Ed., Lippincott, Williams & Wilkins (2005) which is incorporated herein by reference.

“Pharmaceutically acceptable topical carrier” and equivalent terms refer to pharmaceutically acceptable carriers, as described herein above, suitable for topical application. An inactive liquid or cream vehicle capable of suspending or dissolving the active agent(s), and having the properties of being nontoxic and non-inflammatory when applied to the skin, nail, hair, claw or hoof is an example of a pharmaceutically-acceptable topical carrier. This term is specifically intended to encompass carrier materials approved for use in topical cosmetics as well.

The term “pharmaceutically acceptable additive” refers to preservatives, antioxidants, fragrances, emulsifiers, dyes and excipients known or used in the field of drug formulation and that do not unduly interfere with the effectiveness of the biological activity of the active agent, and that is sufficiently non-toxic to the host or patient. Additives for topical formulations are well-known in the art, and may be added to the topical composition, as long as they are pharmaceutically acceptable and not deleterious to the epithelial cells or their function. Further, they should not cause deterioration in the stability of the composition. For example, inert fillers, anti-irritants, tackifiers, excipients, fragrances, opacifiers, antioxidants, gelling agents, stabilizers, surfactant, emollients, coloring agents, preservatives, buffering agents, other permeation enhancers, and other conventional components of topical or transdermal delivery formulations as are known in the art.

The terms “enhancement,” “penetration enhancement” or “permeation enhancement” relate to an increase in the permeability of the skin, nail, hair, claw or hoof to a drug, so as to increase the rate at which the drug permeates through the skin, nail, hair, claw or hoof. The enhanced permeation effected through the use of such enhancers can be observed, for example, by measuring the rate of diffusion of the drug through animal skin, nail, hair, claw or hoof using a diffusion cell apparatus. A diffusion cell is described by Merritt et al. Diffusion Apparatus for Skin Penetration, J of Controlled Release, 1 (1984) pp. 161-162. The term “permeation enhancer” or “penetration enhancer” intends an agent or a mixture of agents, which, alone or in combination, act to increase the permeability of the skin, nail, hair or hoof to a drug.

The term “excipients” is conventionally known to mean carriers, diluents and/or vehicles used in formulating drug compositions effective for the desired use.

The term “topical administration” refers to the application of a pharmaceutical agent to the external surface of the skin, nail, hair, claw or hoof, such that the agent crosses the external surface of the skin, nail, hair, claw or hoof and enters the underlying tissues. Topical administration includes application of the composition to intact skin, nail, hair, claw or hoof, or to a broken, raw or open wound of skin, nail, hair, claw or hoof. Topical administration of a pharmaceutical agent can result in a limited distribution of the agent to the skin and surrounding tissues or, when the agent is removed from the treatment area by the bloodstream, can result in systemic distribution of the agent.

The term “transdermal delivery” refers to the diffusion of an agent across the barrier of the skin, nail, hair, claw or hoof resulting from topical administration or other application of a composition. The stratum corneum acts as a barrier and few pharmaceutical agents are able to penetrate intact skin. In contrast, the epidermis and dermis are permeable to many solutes and absorption of drugs therefore occurs more readily through skin, nail, hair, claw or hoof that is abraded or otherwise stripped of the stratum corneum to expose the epidermis. Transdermal delivery includes injection or other delivery through any portion of the skin, nail, hair, claw or hoof or mucous membrane and absorption or permeation through the remaining portion. Absorption through intact skin, nail, hair, claw or hoof can be enhanced by placing the active agent in an appropriate pharmaceutically acceptable vehicle before application to the skin, nail, hair, claw or hoof. Passive topical administration may consist of applying the active agent directly to the treatment site in combination with emollients or penetration enhancers. As used herein, transdermal delivery is intended to include delivery by permeation through or past the integument, i.e. skin, nail, hair, claw or hoof.

The terms “effective amount” or a “therapeutically effective amount” of a drug or pharmacologically active agent refers to a nontoxic but sufficient amount of the drug or agent to provide the desired effect. In the oral dosage forms of the present disclosure, an “effective amount” of one active of the combination is the amount of that active that is effective to provide the desired effect when used in combination with the other active of the combination. The amount that is “effective” will vary from subject to subject, depending on the age and general condition of the individual, the particular active agent or agents, and the appropriate “effective” amount in any individual case may be determined by one of ordinary skill in the art using routine experimentation.

The phrases “active ingredient”, “therapeutic agent”, “active”, or “active agent” mean a chemical entity which can be effective in treating a targeted disorder, disease or condition.

The phrase “pharmaceutically acceptable” means moieties or compounds that are, within the scope of medical judgment, suitable for use in humans without causing undesirable biological effects such as undue toxicity, irritation, allergic response, and the like, for example.

The phrase “oral dosage form” means any pharmaceutical composition administered to a subject via the oral cavity. Exemplary oral dosage forms include tablets, capsules, films, powders, sachets, granules, solutions, solids, suspensions or as more than one distinct unit (e.g., granules, tablets, and/or capsules containing different actives) packaged together for co-administration, and other formulations known in the art. An oral dosage form can be one, two, three, four, five or six units. When the oral dosage form has multiple units, all of the units are contained within a single package, (e.g. a bottle or other form of packaging such as a blister pack). When the oral dosage form is a single unit, it may or may not be in a single package. In a preferred embodiment, the oral dosage form is one, two or three units. In a particularly preferred embodiment, the oral dosage form is one unit.

The phrase “unit”, as used herein, refers to the number of discrete objects to be administered which comprise the dosage form. In some embodiments, the dosage form includes a compound of the invention in one capsule. This is a single unit. In some embodiments, the dosage form includes a compound of the invention as part of a therapeutically effective dosage of a cream or ointment. This is also a single unit. In some embodiments, the dosage form includes a compound of the invention and another active ingredient contained within one capsule, or as part of a therapeutically effective dosage of a cream or ointment. This is a single unit, whether or not the interior of the capsule includes multiple discrete granules of the active ingredient. In some embodiments, the dosage form includes a compound of the invention in one capsule, and the active ingredient in a second capsule. This is a two unit dosage form, such as two capsules or tablets, and so such units are contained in a single package. Thus the term ‘unit’ refers to the object which is administered to the animal, not to the interior components of the object.

The term, “prodrug”, as defined herein, is a derivative of a parent drug molecule that exerts its pharmacological effect only after chemical and/or enzymatic conversion to its active form in vivo. Prodrugs include those designed to circumvent problems associated with delivery of the parent drug. This may be due to poor physicochemical properties, such as poor chemical stability or low aqueous solubility, and may also be due to poor pharmacokinetic properties, such as poor bioavailability or poor half-life. Thus, certain advantages of prodrugs may include improved chemical stability, absorption, and/or PK properties of the parent carboxylic acids. Prodrugs may also be used to make drugs more “patient friendly,” by minimizing the frequency (e.g., once daily) or route of dosing (e.g., oral), or to improve the taste or odor if given orally, or to minimize pain if given parenterally.

In some embodiments, the prodrugs are chemically more stable than the active drug, thereby improving formulation and delivery of the parent drug, compared to the drug alone.

Prodrugs for carboxylic acid analogs of the invention may include a variety of esters. In an exemplary embodiment, the pharmaceutical compositions of the invention include a carboxylic acid ester. In an exemplary embodiment, the prodrug is suitable for treatment/prevention of those diseases and conditions that require the drug molecule to cross the blood brain barrier. In an exemplary embodiment, the prodrug enters the brain, where it is converted into the active form of the drug molecule. In one embodiment, a prodrug is used to enable an active drug molecule to reach the inside of the eye after topical application of the prodrug to the eye. Additionally, a prodrug can be converted to its parent compound by chemical or biochemical methods in an ex vivo environment. For example, a prodrug can be slowly converted to its parent compound when placed in a transdermal patch reservoir with a suitable enzyme or chemical reagent.

“Antibiotic”, as used herein, is a compound which can kill or inhibit the growth of bacteria. The term antibiotic is broad enough to encompass acids, bases, salt forms (such as pharmaceutically acceptable salts), prodrugs, solvates and hydrates of the antibiotic compound.

“Antiprotozoal” or “antiprotozoa”, as used herein, is a compound which can kill or inhibit the growth of protozoa. The term anti-protozoal or anti-protozoa is broad enough to encompass acids, bases, salt forms (such as pharmaceutically acceptable salts), prodrugs, solvates and hydrates of the antiprotozoal or antiprotozoa compound.

The term “microbial infection” or “infection by a microorganism” refers to any infection of a host by an infectious agent including, but not limited to, viruses, bacteria, mycobacteria, fungus and parasites (see, e.g., Harrison's Principles of Internal Medicine, pp. 93-98 (Wilson et al., eds., 12th ed. 1991); Williams et al., J. of Medicinal Chem. 42:1481-1485 (1999), herein each incorporated by reference in their entirety).

“Biological medium,” as used herein refers to both in vitro and in vivo biological milieus. Exemplary in vitro “biological media” include, but are not limited to, cell culture, tissue culture, homogenates, plasma and blood. In vivo applications are generally performed in mammals, preferably humans.

“Inhibiting” and “blocking,” are used interchangeably herein to refer to the partial or full blockade of an enzyme, such as a beta-lactamase or a leucyl t-RNA synthetase.

Boron is able to form additional covalent or dative bonds with oxygen, sulfur or nitrogen under some circumstances in this invention.

Embodiments of the invention also encompass compounds that are poly- or multi-valent species, including, for example, species such as dimers, trimers, tetramers and higher homologs of the compounds of use in the invention or reactive analogues thereof.

“Salt counterion”, as used herein, refers to positively charged ions that associate with a compound of the invention when the boron is fully negatively or partially negatively charged. Examples of salt counterions include H⁺, H₃O⁺, ammonium, potassium, calcium, magnesium, organic amino (such as choline or diethylamine or amino acids such as d-arginine, 1-arginine, d-lysine, 1-lysine), and sodium.

The compounds comprising a boron bonded to a carbon and three heteroatoms (such as three oxygens described in this section) can optionally contain a fully negatively charged boron or partially negatively charged boron. Due to the negative charge, a positively charged counterion may associate with this compound, thus forming a salt. Examples of positively charged counterions include H⁺, H₃O⁺, ammonium, potassium, calcium, magnesium, organic amino (such as choline or diethylamine or amino acids such as d-arginine, 1-arginine, d-lysine, 1-lysine), and sodium. These salts of the compounds are implicitly contained in descriptions of these compounds.

### II. Introduction

The invention provides novel boron compounds. The novel compounds, as well as pharmaceutical compositions containing such compounds or combinations of these compounds with at least one additional therapeutically effective agent, can be used for, among other things, treating protozoal infections.

### III. The Compounds

### III. a) Cyclic Boronic Esters

In one aspect, the invention provides a compound of the invention. In an exemplary embodiment, the invention is a compound described herein. In an exemplary embodiment, the invention is a compound according to a formula described herein.

In another aspect, the invention provides a compound having a structure according to the following formula:

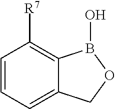

wherein R⁷ is a member selected from any of the possibilities described in this section, or a salt thereof.

In an exemplary embodiment, the compound has a structure according to the following formula:

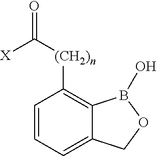

wherein n is 0 or 1 or 2 or 3 or 4 or 5 or 6 or 7 or 8 or 9 or 10, X is selected from the group consisting of substituted or unsubstituted alkyl, OR¹⁰, and NR¹⁰R¹¹, wherein R¹⁰ is H or substituted or unsubstituted alkyl or substituted or unsubstituted aryl and R¹¹ is H or substituted or unsubstituted alkyl, with the proviso that R¹⁰ and R¹¹ along with the nitrogen to which they are attached are optionally combined to form a 4 or 5 or 6 or 7 or 8 membered ring, or a salt thereof. In an exemplary embodiment, n is 0 and X is as described herein. In an exemplary embodiment, n is 1 or 2 or 3 or 4 or 5 or 6 or 7 or 8 or 9 or 10 and X is as described herein. In an exemplary embodiment, n is 1 or 2 or 3 or 4 or 5 and X is as described herein. In an exemplary embodiment, n is as described herein, X is OR¹⁰ and R¹⁰ is methyl or ethyl or propyl or isopropyl or t-butyl or phenyl or benzyl. In an exemplary embodiment, n is as described herein, X is unsubstituted C₁ or C₂ or C₃ or C₄ or C₅ or C₆ alkyl. In an exemplary embodiment, n is 1 or 2 or 3, X is unsubstituted C₁ or C₂ or C₃ or C₄ or C₅ or C₆ alkyl. In an exemplary embodiment, n is 2, X is unsubstituted C₁ or C₂ or C₃ or C₄ or C₅ or C₆ alkyl. In an exemplary embodiment, n is 2, X is unsubstituted C₁ or C₂ alkyl.

In an exemplary embodiment, the compound has a structure according to the following formula:

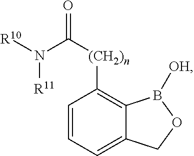

wherein n, R¹⁰ and R¹¹ is as described herein. In an exemplary embodiment, the compound has a structure according to the following formula:

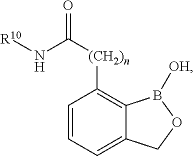

wherein n and R¹⁰ is as described herein. In an exemplary embodiment, n is as described herein, and R¹⁰ is methyl or ethyl or propyl or isopropyl or t-butyl or phenyl or benzyl. In an exemplary embodiment, n is as described herein, and R¹⁰ is ethyl. In an exemplary embodiment, n is 2 and R¹⁰ is ethyl. In an exemplary embodiment, n is as described herein, and R¹⁰ is phenyl. In an exemplary embodiment, n is 2 and R¹⁰ is phenyl. In an exemplary embodiment, n is as described herein, and R¹⁰ is t-butyl. In an exemplary embodiment, n is 2 and R¹⁰ is t-butyl. In an exemplary embodiment, n is as described herein, and R¹⁰ is alkoxyphenyl. In an exemplary embodiment, n is as described herein, and R¹⁰ is methoxyphenyl. In an exemplary embodiment, n is as described herein, and R¹⁰ is p-alkoxyphenyl. In an exemplary embodiment, n is as described herein, and R¹⁰ is p-methoxyphenyl. In an exemplary embodiment, n is 2 and R¹⁰ is p-methoxyphenyl. In an exemplary embodiment, n is as described herein, R¹⁰ is alkoxy-carbonylaminoalkylphenyl. In an exemplary embodiment, n is 2, R¹⁰ is alkoxy-carbonylaminoalkylphenyl. In an exemplary embodiment, n is as described herein, R¹⁰ is butoxy-carbonylaminoalkylphenyl. In an exemplary embodiment, n is as described herein, R¹⁰ is butoxy-carbonylaminomethylphenyl. In an exemplary embodiment, n is 2, R¹⁰ is butoxy-carbonylaminomethylphenyl. In an exemplary embodiment, n is as described herein, R¹⁰ is alkoxy-carbonylaminomethylphenyl. In an exemplary embodiment, n is 2, R¹⁰ is alkoxy-carbonylaminomethylphenyl. In an exemplary embodiment, n is as described herein, and R¹⁰ is aminoalkylphenyl. In an exemplary embodiment, n is as described herein, R¹⁰ is aminomethylphenyl. In an exemplary embodiment, n is 2, R¹⁰ is aminomethylphenyl. In an exemplary embodiment, n is as described herein, and R¹⁰ is imidazolylalkyl. In an exemplary embodiment, n is as described herein and R¹⁰ is imidazolylalkyl. In an exemplary embodiment, n is 2, R¹⁰ is imidazolylpropyl. In an exemplary embodiment, n is as described herein and R¹⁰ is dialkylaminoalkyl. In an exemplary embodiment, n is as described herein and R¹⁰ is (methyl)alkylaminoalkyl. In an exemplary embodiment, n is as described herein and R¹⁰ is dimethylaminoalkyl. In an exemplary embodiment, n is as described herein and R¹⁰ is dialkylaminoethyl. In an exemplary embodiment, n is as described herein and R¹⁰ is dimethylaminoethyl. In an exemplary embodiment, n is 2 and R¹⁰ is dimethylaminoethyl. In an exemplary embodiment, n is as described herein and R¹⁰ is H. In an exemplary embodiment, n is 0 or 1 or 2 or 3 or 4 or 5 or 6 and R¹⁰ is H. In an exemplary embodiment, n is 1 or 2 or 3 and R¹⁰ is H. In an exemplary embodiment, n is 2 and R¹⁰ is H. In an exemplary embodiment, n is 0 or 1 or 2 or 3 or 4 or 5 or 6 and R¹⁰ is S(O)₂R^{10a}, wherein R^{10a} is C₁ or C₂ or C₃ or C₄ or C₅ or C₆ unsubstituted alkyl or C₃ or C₄ or C₅ or C₆ unsubstituted cycloalkyl or NH₂. In an exemplary embodiment, n is 1 or 2 or 3 or 4 or 5 or 6 and R¹⁰ is S(O)₂R^{10a}, wherein R^{10a} is C₁ or C₂ or C₃ or C₄ or C₅ or C₆ unsubstituted alkyl or C₃ or C₄ or C₅ or C₆ unsubstituted cycloalkyl or NH₂. In an exemplary embodiment, n is 1 or 2 or 3 and R¹⁰ is S(O)₂R^{10a}, wherein R^{10a} is C₁ or C₂ or C₃ or C₄ or C₅ or C₆ unsubstituted alkyl or C₃ or C₄ or C₅ or C₆ unsubstituted cycloalkyl or NH₂. In an exemplary embodiment, n is 1 or 2 or 3 and R¹⁰ is S(O)₂R^{10a}, wherein R^{10a} is C₁ or C₂ or C₃ unsubstituted alkyl. In an exemplary embodiment, n is 2 and R¹⁰ is S(O)₂R^{10a}, wherein R^{10a} is C₁ unsubstituted alkyl. In an exemplary embodiment, n is 1 or 2 or 3 or 4 or 5 or 6 and R¹⁰ is S(O)₂R^{10a}, wherein R^{10a} is C₃ or C₄ or C₅ or C₆ unsubstituted cycloalkyl. In an exemplary embodiment, n is 1 or 2 or 3 and R¹⁰ is S(O)₂R^{10a}, wherein R^{10a} is C₃ or C₄ or C₅ or C₆ unsubstituted cycloalkyl. In an exemplary embodiment, n is 2 and R¹⁰ is S(O)₂R^{10a}, wherein R^{10a} is unsubstituted cyclopropyl. In an exemplary embodiment, n is 1 or 2 or 3 or 4 or 5 or 6 and R¹⁰ is S(O)₂NH₂. In an exemplary embodiment, n is 1 or 2 or 3 and R¹⁰ is S(O)₂NH₂. In an exemplary embodiment, n is 2 and R¹⁰ is S(O)₂NH₂.

In an exemplary embodiment, the compound has a structure according to the following formula:

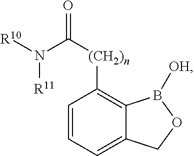

wherein n is as described herein and R¹⁰ and R¹¹ along with the nitrogen to which they are attached are combined to form a 4 or 5 or 6 or 7 or 8 membered ring. In an exemplary embodiment, n is as described herein, and R¹⁰ and R¹¹ along with the nitrogen to which they are attached are combined to form alkylsubstituted or unsubstituted piperazinyl. In an exemplary embodiment, n is as described herein, and R¹⁰ and R¹¹ along with the nitrogen to which they are attached are combined to form methylpiperazinyl. In an exemplary embodiment, n is as described herein, and R¹⁰ and R¹¹ along with the nitrogen to which they are attached are combined to form N-methylpiperazinyl. In an exemplary embodiment, n is 2, and R¹⁰ and R¹¹ along with the nitrogen to which they are attached are combined to form N-methylpiperazinyl.

In an exemplary embodiment, the compound has a structure according to the following formula:

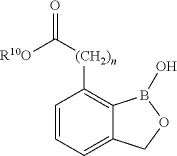

wherein n is 0 or 1 or 2 or 3 or 4 or 5, and R¹⁰ is C₁-C₆ alkyl. In an exemplary embodiment, n is as described herein, R¹⁰ is C₁ alkyl. In an exemplary embodiment, n is as described herein, R¹⁰ is C₂ alkyl. In an exemplary embodiment, n is as described herein, R¹⁰ is C₃ alkyl. In an exemplary embodiment, n is as described herein, R¹⁰ is C₄ alkyl. In an exemplary embodiment, n is as described herein, R¹⁰ is C₅ alkyl. In an exemplary embodiment, n is as described herein, R¹⁰ is C₆ alkyl. In an exemplary embodiment, n is 2 and R¹⁰ is C₂ alkyl. In an exemplary embodiment, n is 0 and R¹⁰ is C₁ alkyl. In an exemplary embodiment, n is 0 and R¹⁰ is as described herein. In an exemplary embodiment, n is 1 and R¹⁰ is as described herein. In an exemplary embodiment, n is 2 and R¹⁰ is as described herein. In an exemplary embodiment, n is 2 and R¹⁰ is C₁ alkyl. In an exemplary embodiment, n is 2 and R¹⁰ is C₃ or C₄ or C₅ or C₆ alkyl.

In an exemplary embodiment, the compound has a structure according to the following formula:

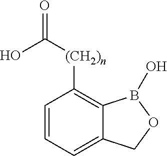

wherein n is 0 or 1 or 2 or 3 or 4 or 5. In an exemplary embodiment, n is 0. In an exemplary embodiment, n is 1. In an exemplary embodiment, n is 2. In an exemplary embodiment, n is 3. In an exemplary embodiment, n is 4. In an exemplary embodiment, n is 5.

In an exemplary embodiment, the compound has a structure according to the following formula:

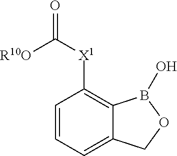

wherein X¹ is halosubstituted C₁ or C₂ or C₃ or C₄ or C₅ or C₆ alkyl or aminosubstituted C₁ or C₂ or C₃ or C₄ or C₅ or C₆ alkyl or hydroxysubstituted C₁ or C₂ or C₃ or C₄ or C₅ or C₆ alkyl and R¹⁰ is C₁ or C₂ or C₃ or C₄ or C₅ or C₆ unsubstituted alkyl. In an exemplary embodiment, X¹ is as described herein, R¹⁰ is C₁ alkyl. In an exemplary embodiment, X¹ is as described herein, R¹⁰ is C₂ alkyl. In an exemplary embodiment, X¹ is as described herein, R¹⁰ is C₃ alkyl. In an exemplary embodiment, X¹ is as described herein, R¹⁰ is C₄ alkyl. In an exemplary embodiment, X¹ is as described herein, R¹⁰ is C₅ alkyl. In an exemplary embodiment, X¹ is as described herein, R¹⁰ is C₆ alkyl. In an exemplary embodiment, X¹ is halosubstituted C₁ or C₂ or C₃ alkyl and R¹⁰ is as described herein. In an exemplary embodiment, X¹ is C₁ or C₂ or C₃ alkyl substituted with one or two halogens and R¹⁰ is as described herein. In an exemplary embodiment, X¹ is C₁ or C₂ or C₃ alkyl substituted with one or two fluorines and R¹⁰ is as described herein. In an exemplary embodiment, X¹ is C₁ or C₂ or C₃ alkyl substituted with two halogens and R¹⁰ is as described herein. In an exemplary embodiment, X¹ is halosubstituted C₂ alkyl and R¹⁰ is C₁ or C₂ or C₃ unsubstituted alkyl. In an exemplary embodiment, X¹ is aminosubstituted C₂ alkyl and R¹⁰ is C₁ or C₂ or C₃ unsubstituted alkyl. In an exemplary embodiment, X¹ is hydroxysubstituted C₁ or C₂ or C₃ alkyl and R¹⁰ is C₁ or C₂ or C₃ unsubstituted alkyl.

In an exemplary embodiment, the compound has a structure according to the following formula:

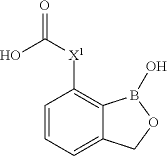

wherein X¹ is halosubstituted C₁ or C₂ or C₃ or C₄ or C₅ or C₆ alkyl or aminosubstituted C₁ or C₂ or C₃ or C₄ or C₅ or C₆ alkyl or hydroxysubstituted C₁ or C₂ or C₃ or C₄ or C₅ or C₆ alkyl. In an exemplary embodiment, X¹ is halosubstituted C₁ or C₂ or C₃ alkyl. In an exemplary embodiment, X¹ is C₁ or C₂ or C₃ alkyl substituted with one or two halogens. In an exemplary embodiment, X¹ is C₁ or C₂ or C₃ alkyl substituted with one or two fluorines. In an exemplary embodiment, X¹ is C₁ or C₂ or C₃ alkyl substituted with two halogens. In an exemplary embodiment, X¹ is halosubstituted C₂ alkyl. In an exemplary embodiment, X¹ is aminosubstituted C₂ alkyl. In an exemplary embodiment, X¹ is hydroxysubstituted C₁ or C₂ or C₃ alkyl.

In an exemplary embodiment, the compound has a structure according to the following formula:

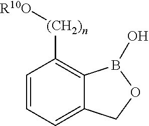

wherein n is 0 or 1 or 2 or 3 or 4 or 5 or 6 or 7 or 8 or 9 or 10, R¹⁰ is H or substituted or unsubstituted alkyl. In an exemplary embodiment, n is 0 and R¹⁰ is H or substituted or unsubstituted alkyl. In an exemplary embodiment, n is 1 or 2 or 3 or 4 or 5 or 6 or 7 or 8 or 9 or 10 and R¹⁰ is H or substituted or unsubstituted alkyl. In an exemplary embodiment, n is 1 or 2 or 3 or 4 or 5 and R¹⁰ is H or substituted or unsubstituted alkyl. In an exemplary embodiment, n is 2 or 3 or 4 or 5 and R¹⁰ is H or substituted or unsubstituted alkyl. In an exemplary embodiment, n is 3 or 4 or 5 and R¹⁰ is H or substituted or unsubstituted alkyl. In an exemplary embodiment, R¹⁰ is H and n is 1 or 5 or 6 or 7 or 8 or 9 or 10. In an exemplary embodiment, n is as described herein, and R¹⁰ is methyl or ethyl or propyl or isopropyl or t-butyl or phenyl or benzyl. In an exemplary embodiment, n is 3 and R¹⁰ is H.

In an exemplary embodiment, the compound has a structure according to the following formula:

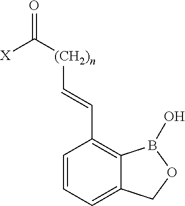

wherein n is 1 or 2 or 3 or 4 or 5 or 6 or 7 or 8 or 9 or 10, X is selected from the group consisting of substituted or unsubstituted alkyl, unsubstituted aryl, OR¹⁰, and NR¹⁰R¹¹, wherein R¹⁰ is H or substituted or unsubstituted alkyl or unsubstituted aryl, R¹¹ is H or substituted or unsubstituted alkyl, or a salt thereof. In an exemplary embodiment, n is as described herein, X is selected from the group consisting of phenyl, OR¹⁰ and N¹⁰R¹¹, wherein R¹⁰ and R¹¹ are as described herein. In an exemplary embodiment, n is as described herein, X is OR¹⁰ and R¹⁰ is methyl or ethyl or propyl or isopropyl or t-butyl or phenyl or benzyl. In an exemplary embodiment, n is as described herein, X is OH. In an exemplary embodiment, n is as described herein, X is ethoxy. In an exemplary embodiment, n is as described herein, X is methoxy. In an exemplary embodiment, n is as described herein, X is OR¹⁰ and R¹⁰ is C₃ or C₄ or C₅ or C₆ alkyl. In an exemplary embodiment, n is as described herein, X is phenyl. In an exemplary embodiment, X is as described herein and n is 1 or 2 or 3 or 4 or 5. In an exemplary embodiment, X is OH and n is 1 or 2 or 3 or 4 or 5. In an exemplary embodiment, X is OH and n is 1 or 2 or 3. In an exemplary embodiment, X is OH and n is 2.

In an exemplary embodiment, the compound has a structure according to the following formula:

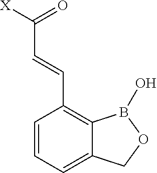

wherein X is selected from the group consisting of substituted or unsubstituted alkyl, unsubstituted aryl, OR¹⁰, and NR¹⁰R¹¹, wherein R¹⁰ is H or substituted or unsubstituted alkyl or unsubstituted aryl, R¹¹ is H or substituted or unsubstituted alkyl, or a salt thereof. In an exemplary embodiment, X is selected from the group consisting of phenyl, OR¹⁰ and N¹⁰R¹¹, wherein R¹⁰ and R¹¹ are as described herein. In an exemplary embodiment, X is OR¹⁰ and R¹⁰ is methyl or ethyl or propyl or isopropyl or t-butyl or phenyl or benzyl. In an exemplary embodiment, X is OH. In an exemplary embodiment, X is ethoxy. In an exemplary embodiment, X is methoxy. In an exemplary embodiment, X is OR¹⁰ and R¹⁰ is C₃ or C₄ or C₅ or C₆ alkyl. In an exemplary embodiment, X is phenyl.

In an exemplary embodiment, the compound has a structure according to the following formula:

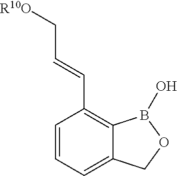

wherein R¹⁰ is H or substituted or unsubstituted alkyl or unsubstituted aryl, or a salt thereof. In an exemplary embodiment, R¹⁰ is H. In an exemplary embodiment, R¹⁰ is C₃ or C₄ or C₅ or C₆ alkyl. In an exemplary embodiment, R¹⁰ is methyl or ethyl or propyl or isopropyl or t-butyl or phenyl or benzyl.

In an exemplary embodiment, the compound has a structure according to the following formula:

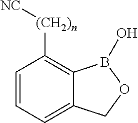

wherein n is 0 or 1 or 2 or 3 or 4 or 5 or 6 or 7 or 8 or 9 or 10, or a salt thereof. In an exemplary embodiment, n is 0. In an exemplary embodiment, n is 1 or 2 or 3 or 4 or 5 or 6 or 7 or 8 or 9 or 10. In an exemplary embodiment, n is 1 or 2 or 3 or 4 or 5. In an exemplary embodiment, n is 1 or 2 or 3. In an exemplary embodiment, n is 2. In an exemplary embodiment, n is 3 or 4 or 5.

In an exemplary embodiment, the compound has a structure according to the following formula:

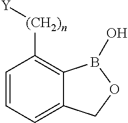

wherein n is 0 or 1 or 2 or 3 or 4 or 5, Y is unsubstituted tetrazolyl, or a salt thereof. In an exemplary embodiment, Y is unsubstituted 1H-tetrazolyl. In an exemplary embodiment, Y is unsubstituted 1H-tetrazol-5-yl. In an exemplary embodiment, Y is as described herein and n is 0. In an exemplary embodiment, Y is as described herein and n is 1 or 2 or 3 or 4 or 5. In an exemplary embodiment, Y is as described herein and n is 1 or 2 or 3. In an exemplary embodiment, Y is as described herein and n is 2. In an exemplary embodiment, Y is as described herein and n is 3 or 4 or 5.

In an exemplary embodiment, the compound has a structure according to the following formula:

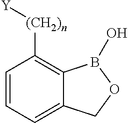

wherein n is 0 or 1 or 2 or 3 or 4 or 5, Y is unsubstituted thiazolidinyl, or a salt thereof. In an exemplary embodiment, n is 0 or 1 or 2 or 3 or 4 or 5, and Y is thiazolidinyl substituted with one or two ketone moieties. In an exemplary embodiment, n is 0 or 1 or 2 or 3 or 4 or 5, and Y is thiazolidinyl 2,4 dione, or a salt thereof. In an exemplary embodiment, Y is as described herein and n is 0. In an exemplary embodiment, Y is as described herein and n is 1 or 2 or 3 or 4 or 5. In an exemplary embodiment, Y is as described herein and n is 1 or 2 or 3. In an exemplary embodiment, Y is as described herein and n is 2. In an exemplary embodiment, Y is as described herein and n is 3 or 4 or 5.

In an exemplary embodiment, the compound has a structure according to the following formula:

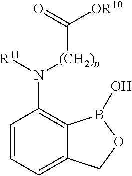

wherein n is 0 or 1 or 2 or 3 or 4 or 5 or 6, R¹⁰ is H or C₁ or C₂ or C₃ or C₄ or C₅ or C₆ unsubstituted alkyl, and R¹¹ is H or C₁ or C₂ or C₃ or C₄ or C₅ or C₆ unsubstituted alkyl or carbonylunsubstituted alkyl, or a salt thereof. In an exemplary embodiment, R¹¹ is H and R¹⁰ and n are as described herein. In an exemplary embodiment, R¹¹ is H and R¹⁰ and n are as described herein. In an exemplary embodiment, R¹¹ is unsubstituted C₁ alkyl and R¹⁰ and n are as described herein. In an exemplary embodiment, R¹¹ is acetyl and R¹⁰ and n are as described herein. In an exemplary embodiment, R¹⁰ is H and R¹¹ and n are as described herein. In an exemplary embodiment, n is 1, R¹⁰ and R¹¹ are as described herein. In an exemplary embodiment, n is 1, R¹⁰ is H and R¹¹ is as described herein. In an exemplary embodiment, n is 1, R¹¹ is H and R¹⁰ are as described herein. In an exemplary embodiment, n is 1, R¹¹ is unsubstituted C₁ alkyl and R¹⁰ are as described herein. In an exemplary embodiment, n is 1, R¹¹ is acetyl and R¹⁰ are as described herein. In an exemplary embodiment, n is 1, R¹¹ is H and R¹⁰ is H. In an exemplary embodiment, n is 1, R¹¹ is unsubstituted C₁ alkyl and R¹⁰ is H. In an exemplary embodiment, n is 1, R¹¹ is acetyl and R¹⁰ is H.

In an exemplary embodiment, the compound has a structure according to the following formula:

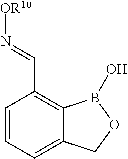

wherein R¹⁰ is H or phenylsubstituted alkyl or unsubstituted alkyl, or a salt thereof. In an exemplary embodiment, R¹⁰ is unsubstituted C₁ alkyl. In an exemplary embodiment, R¹⁰ is C₂ alkyl. In an exemplary embodiment, R¹⁰ is C₃ alkyl. In an exemplary embodiment, R¹⁰ is C₄ alkyl. In an exemplary embodiment, R¹⁰ is C₅ alkyl. In an exemplary embodiment, R¹⁰ is C₆ alkyl. In an exemplary embodiment, R¹⁰ is benzyl.

In an exemplary embodiment, the compound has a structure according to the following formula:

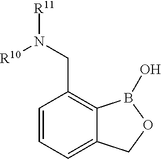

wherein R¹⁰ is H or phenylsubstituted alkyl or unsubstituted alkyl or hydroxysubstituted alkyl or aminosubstituted alkyl or alkylcarbonyl, and R¹¹ is H or phenylsubstituted alkyl or unsubstituted alkyl or hydroxysubstituted alkyl or aminosubstituted alkyl or alkylcarbonyl, wherein R¹⁰ and R¹¹ along with the nitrogen to which they are attached are combined to form a 4 to 8 membered ring, or a salt thereof. In an exemplary embodiment, R¹¹ is H and R¹⁰ is H. In an exemplary embodiment, R¹¹ is H and R¹⁰ is unsubstituted C₁ alkyl. In an exemplary embodiment, R¹¹ is H and R¹⁰ is C₂ alkyl. In an exemplary embodiment, R¹¹ is H and R¹⁰ is C₃ alkyl. In an exemplary embodiment, R¹¹ is H and R¹⁰ is C₄ alkyl. In an exemplary embodiment, R¹¹ is H and R¹⁰ is C₅ alkyl. In an exemplary embodiment, R¹¹ is H and R¹⁰ is C₆ alkyl. In an exemplary embodiment, R¹¹ is H and R¹⁰ is hydroxysubstituted C₁ alkyl. In an exemplary embodiment, R¹¹ is H and R¹⁰ is hydroxysubstituted C₂ alkyl. In an exemplary embodiment, R¹¹ is H and R¹⁰ is 2-hydroxyethyl. In an exemplary embodiment, R¹¹ is H and R¹⁰ is hydroxysubstituted C₃ alkyl. In an exemplary embodiment, R¹¹ is H and R¹⁰ is hydroxysubstituted C₄ alkyl. In an exemplary embodiment, R¹¹ is H and R¹⁰ is hydroxysubstituted C₅ alkyl. In an exemplary embodiment, R¹¹ is H and R¹⁰ is hydroxysubstituted C₆ alkyl. In an exemplary embodiment, R¹¹ is H and R¹⁰ is aminosubstituted C₁ alkyl. In an exemplary embodiment, R¹¹ is H and R¹⁰ is aminosubstituted C₂ alkyl. In an exemplary embodiment, R¹¹ is H and R¹⁰ is 2-aminoethyl. In an exemplary embodiment, R¹¹ is H and R¹⁰ is aminosubstituted C₃ alkyl. In an exemplary embodiment, R¹¹ is H and R¹⁰ is aminosubstituted C₄ alkyl. In an exemplary embodiment, R¹¹ is H and R¹⁰ is aminosubstituted C₅ alkyl. In an exemplary embodiment, R¹¹ is H and R¹⁰ is aminosubstituted C₆ alkyl. In an exemplary embodiment, R¹¹ is H and R¹⁰ is C₁ alkoxy. In an exemplary embodiment, R¹¹ is H and R¹⁰ is C₂ alkoxy. In an exemplary embodiment, R¹¹ is H and R¹⁰ is ethoxy. In an exemplary embodiment, R¹ is H and R¹⁰ is C₃ alkoxy. In an exemplary embodiment, R¹¹ is H and R¹⁰ is C₄ alkoxy. In an exemplary embodiment, R¹¹ is H and R¹⁰ is C₅ alkoxy. In an exemplary embodiment, R¹¹ is H and R¹⁰ is C₆ alkoxy. In an exemplary embodiment, R¹¹ is methyl and R¹⁰ is aminosubstituted C₁ alkyl. In an exemplary embodiment, R¹¹ is methyl and R¹⁰ is aminosubstituted C₂ alkyl. In an exemplary embodiment, R¹¹ is methyl and R¹⁰ is 2-aminoethyl. In an exemplary embodiment, R¹¹ is methyl and R¹⁰ is aminosubstituted C₃ alkyl. In an exemplary embodiment, R¹¹ is methyl and R¹⁰ is aminosubstituted C₄ alkyl. In an exemplary embodiment, R¹¹ is methyl and R¹⁰ is aminosubstituted C₅ alkyl. In an exemplary embodiment, R¹¹ is methyl and R¹⁰ is aminosubstituted C₆ alkyl. In an exemplary embodiment, R¹¹ is methyl and R¹⁰ is cyclobutyl. In an exemplary embodiment, R¹¹ is methyl and R¹⁰ is aminosubstituted cyclopentyl. In an exemplary embodiment, R¹¹ is methyl and R¹⁰ is cyclohexyl. In an exemplary embodiment, R¹¹ is methyl and R¹⁰ is cycloheptanyl. In an exemplary embodiment, R¹¹ is methyl and R¹⁰ is cyclooctanyl. In an exemplary embodiment, R¹¹ is acetyl and R¹⁰ is as described herein. In an exemplary embodiment, R¹¹ is acetyl and R¹⁰ is unsubstituted C₁ alkyl. In an exemplary embodiment, R¹¹ is acetyl and R¹⁰ is unsubstituted C₂ alkyl. In an exemplary embodiment, R¹¹ is acetyl and R¹⁰ is unsubstituted C₃ alkyl. In an exemplary embodiment, R¹¹ is acetyl and R¹⁰ is propyl. In an exemplary embodiment, R¹¹ is acetyl and R¹⁰ is unsubstituted C₄ alkyl. In an exemplary embodiment, R¹¹ is acetyl and R¹⁰ is unsubstituted C₅ alkyl. In an exemplary embodiment, R¹¹ is acetyl and R¹⁰ is unsubstituted C₆ alkyl. In an exemplary embodiment, R¹¹ is acetyl and R¹⁰ is aminosubstituted C₁-C₆ alkyl. In an exemplary embodiment, R¹¹ is acetyl and R¹⁰ is aminoethyl. In an exemplary embodiment, R¹¹ is acetyl and R¹⁰ is N-acetylamino C₁ alkyl. In an exemplary embodiment, R¹¹ is acetyl and R¹⁰ is N-acetylamino C₂ alkyl. In an exemplary embodiment, R¹¹ is acetyl and R¹⁰ is N-acetylamino C₃ alkyl. In an exemplary embodiment, R¹¹ is acetyl and R¹⁰ is N-acetylamino C₄ alkyl. In an exemplary embodiment, R¹¹ is acetyl and R¹⁰ is N-acetylamino C₅ alkyl. In an exemplary embodiment, R¹¹ is acetyl and R¹⁰ is N-acetylamino C₆ alkyl. In an exemplary embodiment, R¹¹ is t-butoxycarbonyl and R¹⁰ is as described herein. In an exemplary embodiment, R¹¹ is t-butoxycarbonyl and R¹⁰ is unsubstituted C₁ alkyl. In an exemplary embodiment, R¹¹ is t-butoxycarbonyl and R¹⁰ is unsubstituted C₂ alkyl. In an exemplary embodiment, R¹¹ is t-butoxycarbonyl and R¹⁰ is unsubstituted C₃ alkyl. In an exemplary embodiment, R¹¹ is t-butoxycarbonyl and R¹⁰ is propyl. In an exemplary embodiment, R¹¹ is t-butoxycarbonyl and R¹⁰ is unsubstituted C₄ alkyl. In an exemplary embodiment, R¹¹ is t-butoxycarbonyl and R¹⁰ is unsubstituted C₅ alkyl. In an exemplary embodiment, R¹¹ is t-butoxycarbonyl and R¹⁰ is unsubstituted C₆ alkyl. In an exemplary embodiment, R¹¹ is t-butoxycarbonyl and R¹⁰ is alkoxysubstituted C₁-C₆ alkyl. In an exemplary embodiment, R¹¹ is t-butoxycarbonyl and R¹⁰ is alkoxyethyl. In an exemplary embodiment, R¹¹ is t-butoxycarbonyl and R¹⁰ is 2-methoxyethyl. In an exemplary embodiment, R¹¹ is t-butoxycarbonyl and R¹⁰ is methoxyalkyl. In an exemplary embodiment, R¹¹ is H and R¹⁰ is alkoxysubstituted C₁-C₆ alkyl. In an exemplary embodiment, R¹¹ is H and R¹⁰ is alkoxyethyl. In an exemplary embodiment, R¹¹ is H and R¹⁰ is 2-methoxyethyl. In an exemplary embodiment, R¹¹ is H and R¹⁰ is methoxyalkyl. In an exemplary embodiment, R¹⁰ and R¹¹ along with the nitrogen to which they are attached are combined to form a 4 to 8 membered ring. In an exemplary embodiment, R¹⁰ and R¹¹ along with the nitrogen to which they are attached are combined to form alkoxycarbonylsubstituted or hydroxyalkylsubstituted or unsubstituted piperidinyl. In an exemplary embodiment, R¹⁰ and R¹¹ along with the nitrogen to which they are attached are combined to form 4-alkyloxycarbonylpiperazinyl. In an exemplary embodiment, R¹⁰ and R¹¹ along with the nitrogen to which they are attached are combined to form ethoxycarbonylpiperazinyl. In an exemplary embodiment, R¹⁰ and R¹¹ along with the nitrogen to which they are attached are combined to form 4-ethoxycarbonylpiperazinyl. In an exemplary embodiment, R¹⁰ and R¹¹ along with the nitrogen to which they are attached are combined to form N-alkyl-piperazinyl. In an exemplary embodiment, R¹⁰ and R¹¹ along with the nitrogen to which they are attached are combined to form N-methylpiperazinyl. In an exemplary embodiment, R¹⁰ and R¹¹ along with the nitrogen to which they are attached are combined to form 4-hydroxyalkylpiperazinyl. In an exemplary embodiment, R¹⁰ and R¹¹ along with the nitrogen to which they are attached are combined to form hydroxymethylpiperazinyl. In an exemplary embodiment, R¹⁰ and R¹¹ along with the nitrogen to which they are attached are combined to form 4-hydroxymethylpiperazinyl. In an exemplary embodiment, R¹⁰ and R¹¹ along with the nitrogen to which they are attached are combined to form hydroxyalkylsubstituted or unsubstituted pyrrolidinyl. In an exemplary embodiment, R¹⁰ and R¹¹ along with the nitrogen to which they are attached are combined to form 4-hydroxyalkylpyrrolidinyl. In an exemplary embodiment, R¹⁰ and R¹¹ along with the nitrogen to which they are attached are combined to form hydroxymethylpyrrolidinyl. In an exemplary embodiment, R¹⁰ and R¹¹ along with the nitrogen to which they are attached are combined to form 2-hydroxymethylpyrrolidinyl. In an exemplary embodiment, R¹⁰ and R¹¹ along with the nitrogen to which they are attached are combined to form 2S-hydroxymethylpyrrolidinyl. In an exemplary embodiment, R¹⁰ and R¹¹ along with the nitrogen to which they are attached are combined to form 2R-hydroxymethylpyrrolidinyl. In an exemplary embodiment, R¹⁰ and R¹¹ along with the nitrogen to which they are attached are combined to form morpholino.

In an exemplary embodiment, the compound of the invention has the following structure:

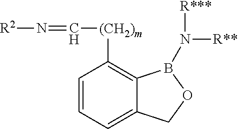

wherein R² is substituted heteroalkyl; m is 0 or 1 or 2 or 3 or 4 or 5 or 6; R** is —C(O)OR²⁰, wherein R²⁰ is unsubstituted alkyl; and R*** is —(CH₂)—NH₂, wherein n is 0or 1 or 2 or 3 or 4 or 5 or 6. In an exemplary embodiment, R² is

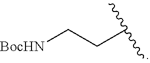

In an exemplary embodiment, m is 0. In an exemplary embodiment, n is 2. In an exemplary embodiment, R²⁰ is C₄ alkyl. In an exemplary embodiment, R²⁰ is t-butyl. In an exemplary embodiment, the compound has a structure which is:

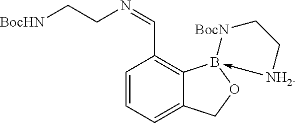

In an exemplary embodiment, the compound has a structure according to the following formula:

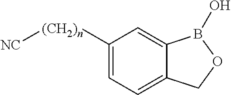

wherein n is 0 or 1 or 2 or 3 or 4 or 5 or 6 or 7 or 8 or 9 or 10, or a salt thereof. In an exemplary embodiment, n is 0. In an exemplary embodiment, n is 1 or 2 or 3 or 4 or 5 or 6 or 7 or 8 or 9 or 10. In an exemplary embodiment, n is 1 or 2 or 3 or 4 or 5. In an exemplary embodiment, n is 1 or 2 or 3. In an exemplary embodiment, n is 2. In an exemplary embodiment, n is 3 or 4 or 5.

In an exemplary embodiment, the compound has a structure according to the following formula:

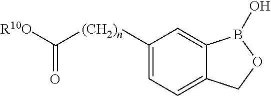

wherein n is 0 or 1 or 2 or 3 or 4 or 5, and R¹⁰ is C₁-C₆ alkyl. In an exemplary embodiment, n is as described herein, R¹⁰ is C₁ alkyl. In an exemplary embodiment, n is as described herein, R¹⁰ is C₂ alkyl. In an exemplary embodiment, n is as described herein, R¹⁰ is C₃ alkyl. In an exemplary embodiment, n is as described herein, R¹⁰ is C₄ alkyl. In an exemplary embodiment, n is as described herein, R¹⁰ is C₅ alkyl. In an exemplary embodiment, n is as described herein, R¹⁰ is C₆ alkyl. In an exemplary embodiment, n is 2 and R¹⁰ is C₂ alkyl. In an exemplary embodiment, n is 0 and R¹⁰ is C₁ alkyl. In an exemplary embodiment, n is 0 and R¹⁰ is as described herein. In an exemplary embodiment, n is 1 and R¹⁰ is as described herein. In an exemplary embodiment, n is 2 and R¹⁰ is as described herein. In an exemplary embodiment, n is 2 and R¹⁰ is C₁ alkyl. In an exemplary embodiment, n is 2 and R¹⁰ is C₃ or C₄ or C₅ or C₆ alkyl.

In an exemplary embodiment, the compound has a structure according to the following formula:

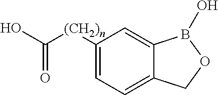

wherein n is 0 or 1 or 2 or 3 or 4 or 5. In an exemplary embodiment, n is 0. In an exemplary embodiment, n is 1. In an exemplary embodiment, n is 2. In an exemplary embodiment, n is 3. In an exemplary embodiment, n is 4. In an exemplary embodiment, n is 5.

In an exemplary embodiment, the compound has a structure according to the following formula:

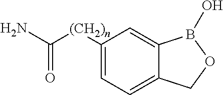

wherein n is 0 or 1 or 2 or 3 or 4 or 5. In an exemplary embodiment, n is 0. In an exemplary embodiment, n is 1. In an exemplary embodiment, n is 2. In an exemplary embodiment, n is 3. In an exemplary embodiment, n is 4. In an exemplary embodiment, n is 5.

In an exemplary embodiment, the compound has a structure according to the following formula:

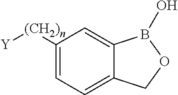

wherein n is 0 or 1 or 2 or 3 or 4 or 5, Y is unsubstituted tetrazolyl, or a salt thereof. In an exemplary embodiment, Y is unsubstituted 1H-tetrazolyl. In an exemplary embodiment, Y is unsubstituted 1H-tetrazol-5-yl. In an exemplary embodiment, Y is as described herein and n is 0. In an exemplary embodiment, Y is as described herein and n is 1 or 2 or 3 or 4 or 5. In an exemplary embodiment, Y is as described herein and n is 1 or 2 or 3. In an exemplary embodiment, Y is as described herein and n is 2. In an exemplary embodiment, Y is as described herein and n is 3 or 4 or 5.

In an exemplary embodiment, the compound has a structure according to the following formula:

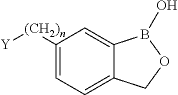

wherein n is 0 or 1 or 2 or 3 or 4 or 5, Y is unsubstituted thiazolidinyl, or a salt thereof. In an exemplary embodiment, n is 0 or 1 or 2 or 3 or 4 or 5, and Y is thiazolidinyl substituted with one or two ketone moieties. In an exemplary embodiment, n is 0 or 1 or 2 or 3 or 4 or 5, and Y is thiazolidinyl 2,4 dione, or a salt thereof. In an exemplary embodiment, Y is as described herein and n is 0. In an exemplary embodiment, Y is as described herein and n is 1 or 2 or 3 or 4 or 5. In an exemplary embodiment, Y is as described herein and n is 1 or 2 or 3. In an exemplary embodiment, Y is as described herein and n is 2. In an exemplary embodiment, Y is as described herein and n is 3 or 4 or 5.

In an exemplary embodiment, the compound has a structure according to the following formula:

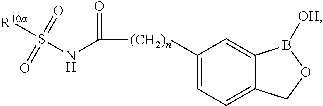

wherein n is 0 or 1 or 2 or 3 or 4 or 5 or 6 and R^{10a} is C₁ or C₂ or C₃ or C₄ or C₅ or C₆ unsubstituted alkyl or C₃ or C₄ or C₅ or C₆ unsubstituted cycloalkyl or NH₂. In an exemplary embodiment, n is 1 or 2 or 3 or 4 or 5 or 6 and R^{10a} is C₁ or C₂ or C₃ or C₄ or C₅ or C₆ unsubstituted alkyl or C₃ or C₄ or C₅ or C₆ unsubstituted cycloalkyl or NH₂. In an exemplary embodiment, n is 1 or 2 or 3 and R^{10a} is C₁ or C₂ or C₃ or C₄ or C₅ or C₆ unsubstituted alkyl or C₃ or C₄ or C₅ or C₆ unsubstituted cycloalkyl or NH₂. In an exemplary embodiment, n is 1 or 2 or 3 and R^{10a} is C₁ or C₂ or C₃ unsubstituted alkyl. In an exemplary embodiment, n is 2 and R^{10a} is C₁ unsubstituted alkyl. In an exemplary embodiment, n is 1 or 2 or 3 or 4 or 5 or 6 and R^{10a} is C₃ or C₄ or C₅ or C₆ unsubstituted cycloalkyl. In an exemplary embodiment, n is 1 or 2 or 3 and R^{10a} is C₃ or C₄ or C₅ or C₆ unsubstituted cycloalkyl. In an exemplary embodiment, n is 2 and R^{10a} is unsubstituted cyclopropyl. In an exemplary embodiment, n is 1 or 2 or 3 or 4 or 5 or 6 and R^{10a} is NH₂. In an exemplary embodiment, n is 1 or 2 or 3 and R^{10a} is NH₂. In an exemplary embodiment, n is 2 and R^{10a} is NH₂.

In an exemplary embodiment, the compound has a structure according to the following formula:

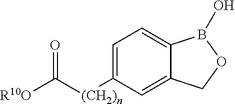

wherein n is 0 or 1 or 2 or 3 or 4 or 5, and R¹⁰ is C₁-C₆ alkyl. In an exemplary embodiment, n is as described herein, R¹⁰ is C₁ alkyl. In an exemplary embodiment, n is as described herein, R¹⁰ is C₂ alkyl. In an exemplary embodiment, n is as described herein, R¹⁰ is C₃ alkyl. In an exemplary embodiment, n is as described herein, R¹⁰ is C₄ alkyl. In an exemplary embodiment, n is as described herein, R¹⁰ is C₅ alkyl. In an exemplary embodiment, n is as described herein, R¹⁰ is C₆ alkyl. In an exemplary embodiment, n is 2 and R¹⁰ is C₂ alkyl. In an exemplary embodiment, n is 0 and R¹⁰ is C₁ alkyl. In an exemplary embodiment, n is 0 and R¹⁰ is as described herein. In an exemplary embodiment, n is 1 and R¹⁰ is as described herein. In an exemplary embodiment, n is 2 and R¹⁰ is as described herein. In an exemplary embodiment, n is 2 and R¹⁰ is C₁ alkyl. In an exemplary embodiment, n is 2 and R¹⁰ is C₃ or C₄ or C₅ or C₆ alkyl.

In an exemplary embodiment, the compound has a structure according to the following formula:

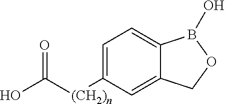

wherein n is 0 or 1 or 2 or 3 or 4 or 5. In an exemplary embodiment, n is 0. In an exemplary embodiment, n is 1. In an exemplary embodiment, n is 2. In an exemplary embodiment, n is 3. In an exemplary embodiment, n is 4. In an exemplary embodiment, n is 5.

In an exemplary embodiment, the compound has a structure according to the following formula:

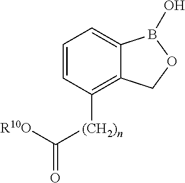

wherein n is 0 or 1 or 2 or 3 or 4 or 5, and R¹⁰ is C₁-C₆ alkyl. In an exemplary embodiment, n is as described herein, R¹⁰ is C₁ alkyl. In an exemplary embodiment, n is as described herein, R¹⁰ is C₂ alkyl. In an exemplary embodiment, n is as described herein, R¹⁰ is C₃ alkyl. In an exemplary embodiment, n is as described herein, R¹⁰ is C₄ alkyl. In an exemplary embodiment, n is as described herein, R¹⁰ is C₅ alkyl. In an exemplary embodiment, n is as described herein, R¹⁰ is C₆ alkyl. In an exemplary embodiment, n is 2 and R¹⁰ is C₂ alkyl. In an exemplary embodiment, n is 0 and R¹⁰ is C₁ alkyl. In an exemplary embodiment, n is 0 and R¹⁰ is as described herein. In an exemplary embodiment, n is 1 and R¹⁰ is as described herein. In an exemplary embodiment, n is 2 and R¹⁰ is as described herein. In an exemplary embodiment, n is 2 and R¹⁰ is C₁ alkyl. In an exemplary embodiment, n is 2 and R¹⁰ is C₃ or C₄ or C₅ or C₆ alkyl.

In an exemplary embodiment, the compound has a structure according to the following formula:

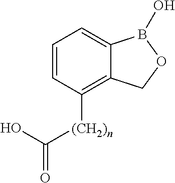

wherein n is 0 or 1 or 2 or 3 or 4 or 5. In an exemplary embodiment, n is 0. In an exemplary embodiment, n is 1. In an exemplary embodiment, n is 2. In an exemplary embodiment, n is 3. In an exemplary embodiment, n is 4. In an exemplary embodiment, n is 5.

In an exemplary embodiment, the compound has a structure according to the following formula:

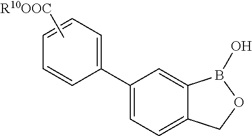

wherein R¹⁰ is C₁-C₆ alkyl. In an exemplary embodiment, R¹⁰ is C₁ alkyl. In an exemplary embodiment, n is as described herein, R¹⁰ is C₂ alkyl. In an exemplary embodiment, n is as described herein, R¹⁰ is C₃ alkyl. In an exemplary embodiment, n is as described herein, R¹⁰ is C₄ alkyl. In an exemplary embodiment, n is as described herein, R¹⁰ is C₅ alkyl. In an exemplary embodiment, n is as described herein, R¹⁰ is C₆ alkyl. In an exemplary embodiment, the compound has a structure which is

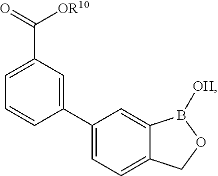

wherein R¹⁰ is as described herein.

In an exemplary embodiment, the compound has a structure according to the following formula:

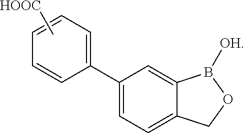

In an exemplary embodiment, the compound has a structure which is

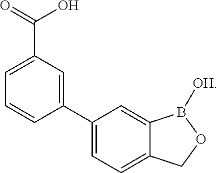

In an exemplary embodiment, alkyl is linear alkyl. In another exemplary embodiment, alkyl is branched alkyl.

In an exemplary embodiment, heteroalkyl is linear heteroalkyl. In another exemplary embodiment, heteroalkyl is branched heteroalkyl.

In an exemplary embodiment, the invention provides a compound described herein, or a salt, hydrate or solvate thereof, or a combination thereof. In an exemplary embodiment, the invention provides a compound described herein, or a salt, hydrate or solvate thereof. In an exemplary embodiment, the invention provides a compound described herein, or a salt thereof. In an exemplary embodiment, the salt is a pharmaceutically acceptable salt. In an exemplary embodiment, the invention provides a compound described herein, or a hydrate thereof. In an exemplary embodiment, the invention provides a compound described herein, or a solvate thereof. In an exemplary embodiment, the invention provides a compound described herein, or a prodrug thereof. In an exemplary embodiment, the invention provides a salt of a compound described herein. In an exemplary embodiment, the invention provides a pharmaceutically acceptable salt of a compound described herein. In an exemplary embodiment, the invention provides a hydrate of a compound described herein. In an exemplary embodiment, the invention provides a solvate of a compound described herein. In an exemplary embodiment, the invention provides a prodrug of a compound described herein.

### III.b) Compositions Involving Stereoisomers

As used herein, the term “chiral”, “enantiomerically enriched” or “diastereomerically enriched” refers to a composition having an enantiomeric excess (ee) or a diastereomeric excess (de) of greater than about 50%, preferably greater than about 70% and more preferably greater than about 90%. In general, higher than about 90% enantiomeric or diastereomeric excess is particularly preferred, e.g., those compositions with greater than about 95%, greater than about 97% and greater than about 99% ee or de.

When a first compound and a second compound are present in a composition, and the first compound is a non-superimposable mirror image of the second compound, and the first compound is present in the composition in a greater amount than the second compound, then the first compound is referred to herein as being present in “enantiomeric excess”.

The term “enantiomeric excess” of a compound z, as used herein, is defined as:

$${ee}_{z} = ( \frac{conc . of z - conc . of y}{conc . of z + conc . of y} ) \times 100$$

wherein z is a first compound in a composition, y is a second compound in the composition, and the first compound is a non-superimposable mirror image of the second compound.

The term “enantiomeric excess” is related to the older term “optical purity” in that both are measures of the same phenomenon. The value of ee will be a number from 0 to 100, zero being racemic and 100 being enantiomerically pure. A composition which in the past might have been called 98% optically pure is now more precisely characterized by 96% ee. A 90% ee reflects the presence of 95% of one enantiomer and 5% of the other(s) in the material in question.

When a first compound and at least one additional compound are present in a composition, and the first compound and each of the additional compounds are stereoisomers, but not mirror images, of one another, and the first compound is present in the composition in a greater amount than each of the additional compounds, then the first compound is referred to herein as being present in “diastereomeric excess”.

When dealing with mixtures of diastereomers, the term “diastereomeric excess” or “de” is defined analogously to enantiomeric excess. Thus:

$${de}_{w} = ( \frac{conc . of major diasteomer - conc . of minor diastereomer ( s )}{conc . of major diasteomer + conc . of minor diastereomer ( s )} ) \times 100$$

wherein the major diastereomer is a first compound in a composition, and the minor diastereomer(s) is at least one additional compound in the composition, and the major diastereomer and minor diastereomer(s) are stereoisomers, but not mirror images, of one another.

The value of de will likewise be a number from 0 to 100, zero being an equal mixture of a first diastereomer and the remaining diastereomer(s), and 100 being 100% of a single diastereomer and zero % of the other(s)—i.e. diastereomerically pure. Thus, 90% de reflects the presence of 95% of one diastereomer and 5% of the other diastereomer(s) in the material in question.

Hence, in one embodiment, the invention provides a composition including a first compound of the invention, wherein the first compound of the invention has at least one stereocenter, and at least one stereoisomer of the first compound of the invention. In another embodiment, the invention provides a composition including a first compound of the invention, wherein the first compound of the invention has at least one stereocenter, and a second compound of the invention, wherein the first compound of the invention is a stereoisomer of the second compound of the invention. In another embodiment, the invention provides a composition including a first compound of the invention, wherein the first compound of the invention has at least one stereocenter, and only one stereoisomer of the first compound of the invention.

In another embodiment, the invention provides a composition including a first compound of the invention, wherein the first compound of the invention has only one stereocenter, and an enantiomer of the first compound of the invention. In another embodiment, the invention provides a composition including a first compound of the invention, wherein the first compound of the invention has two stereocenters, and an enantiomer of the first compound of the invention. In another embodiment, the invention provides a composition including a first compound of the invention, wherein the first compound of the invention has two stereocenters, and at least one diastereomer of the first compound of the invention. In another embodiment, the invention provides a composition including a first compound of the invention, wherein the first compound of the invention has two stereocenters, and only one diastereomer of the first compound of the invention.

In situations where the first compound of the invention and its enantiomer are present in a composition, the first compound of the invention can be present in an enantiomeric excess of at least about 80%, or at least about 90%, or at least about 92% or at least about 95%. In another embodiment, where the first compound of the invention and its enantiomer are present in a composition, the first compound of the invention can be present in an enantiomeric excess of at least about 96%, at least about 97%, at least about 98%, at least about 99% or at least about 99.5%. In another embodiment, the first compound of the invention has at least one stereocenter and is enantiomerically pure (enantiomeric excess is about 100%).

In situations where the first compound of the invention and at least one diastereomer of the first compound of the invention are present in a composition, the first compound of the invention can be present in a diastereomeric excess of at least about 80%, or at least about 90%, or at least about 92% or at least about 95%. In situations where the first compound of the invention and at least one diastereomer of the first compound of the invention are present in a composition, the first compound of the invention can be present in a diastereomeric excess of at least about 96%, at least about 97%, at least about 98%, at least about 99% or at least about 99.5%. In another embodiment, the first compound of the invention has at least two stereocenters and is diastereomerically pure (diastereomeric excess is about 100%).

Enantiomeric or diastereomeric excess can be determined relative to exactly one other stereoisomer, or can be determined relative to the sum of at least two other stereoisomers. In an exemplary embodiment, enantiomeric or diastereomeric excess is determined relative to all other detectable stereoisomers, which are present in the mixture. Stereoisomers are detectable if a concentration of such stereoisomer in the analyzed mixture can be determined using common analytical methods, such as chiral HPLC.

As used herein, and unless otherwise indicated, a composition that is “substantially free” of a compound means that the composition contains less than about 20% by weight, or less than about 15% by weight, or less than about 10% by weight, or less than about 5% by weight, or less than about 3% by weight, or less than about 2% by weight, or less than about 1% by weight of the compound.

As used herein, the term “substantially free of the (or its) enantiomer” means that a composition contains a significantly greater proportion of a first compound of the invention than a second compound of the invention, wherein the first compound is a non-superimposable mirror image of the second compound. In one embodiment of the invention, the term “substantially free of the enantiomer” means that the composition is made up of at least about 90% by weight of a first compound of the invention, and about 10% by weight or less of a second compound of the invention, wherein the first compound is a non-superimposable mirror image of the second compound. In one embodiment of the invention, the term “substantially free of the (R) enantiomer” means that the composition is made up of at least about 90% by weight of a first compound of the invention which has only one stereocenter and the stereocenter is in an (S) configuration, and about 10% by weight or less of a second compound of the invention, wherein the second compound is the enantiomer of the first compound. In one embodiment of the invention, the term “substantially free of the enantiomer” means that the composition is made up of at least about 95% by weight of a first compound of the invention, and about 5% by weight or less of a second compound of the invention, wherein the first compound is a non-superimposable mirror image of the second compound. In one embodiment of the invention, the term “substantially free of the (R) enantiomer” means that the composition is made up of at least about 95% by weight of a first compound of the invention which has only one stereocenter and the stereocenter is in an (S) configuration, and about 5% by weight or less of a second compound of the invention, wherein the second compound is the enantiomer of the first compound. In one embodiment of the invention, the term “substantially free of the enantiomer” means that the composition is made up of at least about 98% by weight of a first compound of the invention, and about 2% by weight or less of a second compound of the invention, wherein the first compound is a non-superimposable mirror image of the second compound. In one embodiment of the invention, the term “substantially free of the (R) enantiomer” means that the composition is made up of at least about 98% by weight of a first compound of the invention which has only one stereocenter and the stereocenter is in an (S) configuration, and about 2% by weight or less of a second compound of the invention, wherein the second compound is the enantiomer of the first compound. In one embodiment of the invention, the term “substantially free of the enantiomer” means that the composition is made up of at least about 99% by weight of a first compound of the invention, and about 1% by weight or less of a second compound of the invention, wherein the first compound is a non-superimposable mirror image of the second compound. In one embodiment of the invention, the term “substantially free of the (R) enantiomer” means that the composition is made up of at least about 99% by weight of a first compound of the invention which has only one stereocenter and the stereocenter is in an (S) configuration, and about 1% by weight or less of a second compound of the invention, wherein the second compound is the enantiomer of the first compound.

In an exemplary embodiment, the invention provides a composition comprising a) first compound described herein; and b) the enantiomer of the first compound, wherein the first compound described herein is present in an enantiomeric excess of at least 80%. In an exemplary embodiment, the enantiomeric excess is at least 92%.

### III.b) Combinations Comprising Additional Therapeutic Agents

The compounds of the invention may also be used in combination with additional therapeutic agents. The invention thus provides, in a further aspect, a combination comprising a compound described herein or a pharmaceutically acceptable salt thereof together with at least one additional therapeutic agent. In an exemplary embodiment, the additional therapeutic agent is a compound of the invention. In an exemplary embodiment, the additional therapeutic agent includes a boron atom. In an exemplary embodiment, the additional therapeutic agent does not contain a boron atom.

When a compound of the invention is used in combination with a second therapeutic agent active against the same disease state, the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. In an exemplary embodiment, the additional therapeutic agent is berenil. In an exemplary embodiment, the additional therapeutic agent is diminazene. In an exemplary embodiment, the additional therapeutic agent is an antiprotozoa. In an exemplary embodiment, the additional therapeutic agent is selected from the group consisting of benznidazole, buparvaquone, carbarsone, clioquinol, disulfiram, eflornithine, emetine, etofamide, furazolidone, meglumine antimoniate, melarsoprol, metronidazole, miltefosine, nifurtimox, nimorazole, nitazoxanide, ornidazole, paromomycin sulfate, pentamidine, pyrimethamine, secnidazole and tinidazole. In an exemplary embodiment, the additional therapeutic agent is pentamidine. In an exemplary embodiment, the additional therapeutic agent is suramin. In an exemplary embodiment, the additional therapeutic agent is eflornithine. In an exemplary embodiment, the additional therapeutic agent is melarsoprol. In an exemplary embodiment, the additional therapeutic agent is nifurtimox. In an exemplary embodiment, the additional therapeutic agent contains a 5-nitrofuran moiety. In an exemplary embodiment, the additional therapeutic agent contains a 5-nitroimidazolyl moiety. In an exemplary embodiment, the additional therapeutic agent is fexinidazole. In an exemplary embodiment, the additional therapeutic agent is an antiparasitic. In an exemplary embodiment, the additional therapeutic agent is selected from the group consisting of amitraz, avermectin, carbadox, diethylcarbamazine, dimetridazole, diminazene, ivermectin, macrofilaricide, malathion, mitaban, organophosphate, oxamniquine, permethrin, praziquantel, pyrantel pamoate, selamectin, sodium stibogluconate and thiabendazole. In an exemplary embodiment, the additional therapeutic agent is selected from the group consisting of antimony, meglumine antimoniate, sodium stibogluconate, amphotericin, miltefosine and paromomycin.

In an exemplary embodiment, the additional therapeutic agent is an antimalarial. In an exemplary embodiment, the additional therapeutic agent is artemisinin. In an exemplary embodiment, the additional therapeutic agent is an artemisinin derivative. In an exemplary embodiment, the additional therapeutic agent is an artemisinin derivative which is artesunate or artemether or artemotil or dihydroartemisinin. In an exemplary embodiment, the additional therapeutic agent is a member selected from lumefantrine, artemether-lumefantrine, amodiaquine, artesunate-amodiaquine, artesunate-mefloquine, artesunate-sulfadoxine/pyrimethamine, atovaquone-proguanil, quinine, chloroquine, cotrifazid, doxycycline, mefloquine, primaquine, proguanil, sulfadoxine-pyrimethamine, hydroxychloroquine, sulfalene-pyrimethamine, dapsone, proguanil-dapsone and chloroproguanil-dapsone. In an exemplary embodiment, the additional therapeutic agent is a member selected from amodiaquine, chloroquine and sulfadoxine-pyrimethamine. In an exemplary embodiment, the additional therapeutic agent is mefloquine. In an exemplary embodiment, the additional therapeutic agent is a member selected from halofantrine, dihydroartemisinin-piperaquine, piperaquine, pyronaridine and tetracycline.

The compounds of the invention, or pharmaceutical formulations thereof may also be used in combination with other therapeutic agents, for example immune therapies [e.g. interferon, such as interferon alfa-2a (ROFERON®-A; Hoffmann-La Roche), interferon alpha-2b (INTRON®-A; Schering-Plough), interferon alfacon-1 (INFERGEN®; Intermune), peginterferon alpha-2b (PEGINTRON™; Schering-Plough) or peginterferon alpha-2a (PEGASYS®; Hoffmann-La Roche)], therapeutic vaccines, antifibrotic agents, anti-inflammatory agents [such as corticosteroids or NSAIDs], bronchodilators [such as beta-2 adrenergic agonists and xanthines (e.g. theophylline)], mucolytic agents, anti-muscarinics, anti-leukotrienes, inhibitors of cell adhesion [e.g. ICAM antagonists], anti-oxidants [e.g. N-acetylcysteine], cytokine agonists, cytokine antagonists, lung surfactants and/or antimicrobial. The compositions according to the invention may also be used in combination with gene replacement therapy.

The individual components of such combinations may be administered either simultaneously or sequentially in a unit dosage form. The unit dosage form may be a single or multiple unit dosage forms. In an exemplary embodiment, the invention provides a combination in a single unit dosage form. An example of a single unit dosage form is a capsule wherein both the compound of the invention and the additional therapeutic agent are contained within the same capsule. In an exemplary embodiment, the invention provides a combination in a two unit dosage form. An example of a two unit dosage form is a first capsule which contains the compound of the invention and a second capsule which contains the additional therapeutic agent. Thus the term ‘single unit’ or ‘two unit’ or ‘multiple unit’ refers to the object which the patient ingests, not to the interior components of the object. Appropriate doses of known therapeutic agents will be readily appreciated by those skilled in the art.

The combinations referred to herein may conveniently be presented for use in the form of a pharmaceutical formulation. Thus, an exemplary embodiment of the invention is a pharmaceutical formulation comprising a) a compound of the invention; b) an additional therapeutic agent and c) a pharmaceutically acceptable excipient. In an exemplary embodiment, the pharmaceutical formulation is a unit dosage form. In an exemplary embodiment, the pharmaceutical formulation is a single unit dosage form. In an exemplary embodiment, the pharmaceutical formulation is a two unit dosage form. In an exemplary embodiment, the pharmaceutical formulation is a two unit dosage form comprising a first unit dosage form and a second unit dosage form, wherein the first unit dosage form includes a) a compound of the invention and b) a first pharmaceutically acceptable excipient; and the second unit dosage form includes c) an additional therapeutic agent and d) a second pharmaceutically acceptable excipient.

It is to be understood that the invention covers all combinations of aspects and/or embodiments, as well as suitable, convenient and preferred groups described herein.

### III.c) Preparation of Boron-Containing Compounds

Compounds of use in the invention can be prepared using commercially available starting materials, known intermediates, or by using the synthetic methods described herein, or published in references described and incorporated by reference herein, such as U.S. Prov. Pat. App. 60/654,060; Filed Feb. 16, 2005 (Attorney Docket No. 064507-5014PR); U.S. patent application Ser. No. 11/357,687, Filed Feb. 16, 2006 (Attorney Docket No. 064507-5014US); U.S. patent application Ser. No. 11/505,591, Filed Aug. 16, 2006 (Attorney Docket No. 064507-5014US01), U.S. Prov. Pat. App. 60/823,888 filed on Aug. 29, 2006 and 60/774,532 filed on Feb. 16, 2006 (Attorney Docket No. 064507-5016PR and 064507-5016PR01, respectively); U.S. patent application Ser. No. 11/676,120, Filed Feb. 16, 2007 (Attorney Docket No. 064507-5016US); U.S. patent application Ser. No. 12/142,692, Filed Jun. 19, 2008 (Attorney Docket No. 064507-5026US); U.S. patent application Ser. No. 12/399,015, Filed Mar. 5, 2009 (Attorney Docket No. 064507-5029US); U.S. patent application Ser. No. 12/464,829, Filed May 12, 2009 (Attorney Docket No. 064507-5033US); which are herein incorporated by reference in their entirety for all purposes. Methods of producing the compounds of the invention are also described in these patent applications.

The compounds in this invention can be prepared as shown in the reactions schemes below. To make the α, β-unsaturated carbonyl derivatives C, aldehyde A can be reacted under basic conditions (such as those shown below) with an aromatic ketone B (Z¹=alkyl) as shown below.

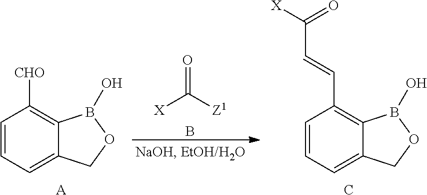

Unsaturated esters such as E can be prepared by a standard Wittig reaction with aldehyde A and alkyl halide D, while the saturated esters F can be obtained by subjecting E to reducing conditions, such as catalytic reduction of E with hydrogen in the presence of platinum oxide catalyst.

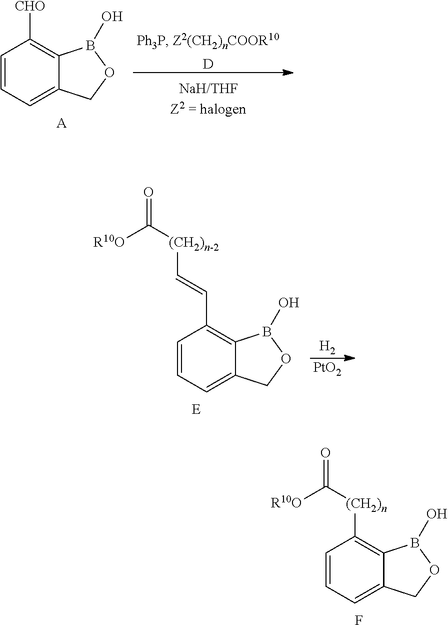

The corresponding carboxylic acids, such as G, can be obtained by subjecting F to hydrolysis conditions such as those shown below.

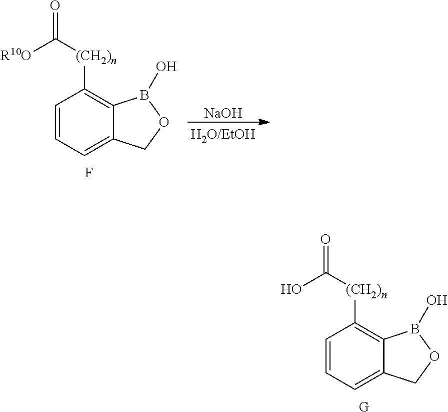

Amides such as H can be prepared from the corresponding acids by standard peptide coupling conditions as shown below.

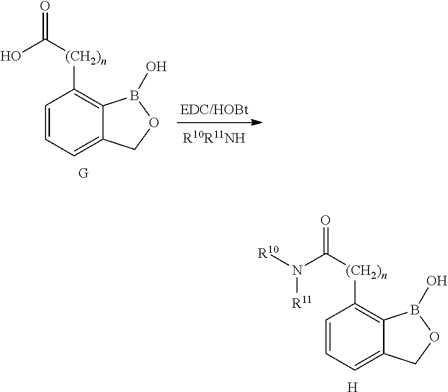

Primary alcohols such as I can be prepared by reduction of the ester F with DIBAH.

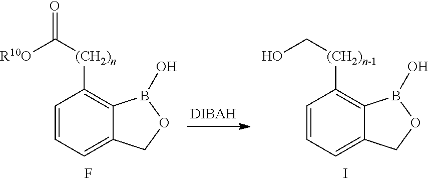

Oxime ethers J can be prepared by reaction of aldehyde A with hydroxylamine ethers as shown below.

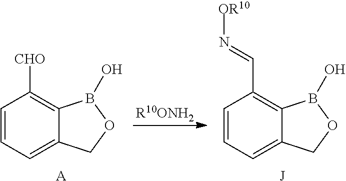

Aminomethyl derivatives K can be prepared by subjecting A to reductive amination conditions such as sodium triacetoxy borohydride and an appropriate amine.

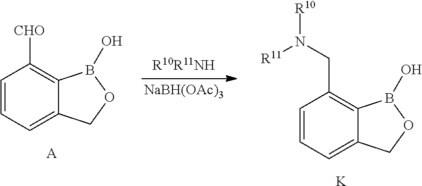

Compounds described herein can be converted into hydrates and solvates by methods similar to those described herein.

### IV. Methods of Inhibiting Microorganism Growth or Killing Microorganisms

The compounds of the invention exhibit potency against microorganisms, such as protozoa, and therefore have the potential to kill and/or inhibit the growth of microorganisms.

In a further aspect, the invention provides a method of killing and/or inhibiting the growth of a microorganism, said method comprising: contacting said microorganism with an effective amount of a compound of the invention, thereby killing and/or inhibiting the growth of the microorganism. In an exemplary embodiment, the microorganism is a protozoa. In an exemplary embodiment, the microorganism is a kinetoplastid. In another exemplary embodiment, the protozoa is a Trypanosoma. In an exemplary embodiment, the Trypanosoma is a member selected from T. avium, T. boissoni, T. brucei, T carassii, T cruzi, T congolense, T. equinum, T. equiperdum, T. evansi, T hosei, T. levisi, T. melophagium, T. parroti, T. percae, T. rangeli, T rotatorium, T. rugosae, T. sergenti, T. simiae, T. sinipercae, T. suis, T. theileri, T. triglae and T. vivax. In another exemplary embodiment, the protozoa is a Trypanosoma brucei. In another exemplary embodiment, the protozoa is a member selected from Trypanosoma brucei brucei, Trypanosoma brucei rhodesiense and Trypanosoma brucei gambiense. In another exemplary embodiment, the protozoa is a member selected from Trypanosoma brucei rhodesiense and Trypanosoma brucei gambiense. In another exemplary embodiment, the protozoa is Trypanosoma cruzi. In another exemplary embodiment, the protozoa is a member of the genus Leishmania. In another exemplary embodiment, the protozoa is a member of Leishmania Viannia. In an exemplary embodiment, the protozoa is a member selected from L. donovani, L. infantum, L. chagasi; L. mexicana, L. amazonensis, L. venezuelensis, L. tropica, L. major, L. aethiopica, L. (V.) braziliensis, L. (V.) guyanensis, L. (V.) panamensis, and L. (V.) peruviana. In an exemplary embodiment, the protozoa is L. donovani. In an exemplary embodiment, the protozoa is L. infantum. In another exemplary embodiment, the protozoa is a member of the genus Plasmodium. In another exemplary embodiment, the protozoa is a member selected from Plasmodium falciparum, Plasmodium vivax, Plasmodium ovale, Plasmodium vivax, Plasmodium malariae and Plasmodium knowlesi. In another exemplary embodiment, the protozoa is a member selected from Plasmodium vivax, Plasmodium ovale, Plasmodium vivax and Plasmodium malariae. In another exemplary embodiment, the protozoa is Plasmodium falciparum. In another exemplary embodiment, the protozoa is transmitted to the animal described herein by a mosquito infected with the protozoa. In another exemplary embodiment, wherein the protozoa is transmitted to the animal described herein by an Anopheles mosquito containing the protozoa. In an exemplary embodiment, the compound is described herein, or a salt, prodrug, hydrate or solvate thereof, or a combination thereof. In an exemplary embodiment, the invention provides a compound described herein, or a salt, hydrate or solvate thereof. In an exemplary embodiment, the invention provides a compound described herein, or a prodrug thereof. In an exemplary embodiment, the invention provides a compound described herein, or a salt thereof. In another exemplary embodiment, the compound of the invention is a compound described herein, or a pharmaceutically acceptable salt thereof. In another exemplary embodiment, the compound is described by a formula listed herein, or a pharmaceutically acceptable salt thereof. In an exemplary embodiment, the compound is part of a pharmaceutical formulation described herein. In another exemplary embodiment, the contacting occurs under conditions which permit entry of the compound into the organism. In another exemplary embodiment, the compound of the invention is 7-(2-carboxyethyl)-1,3-dihydro-1-hydroxy-2,1-benzoxaborole, or a salt thereof. Such conditions are known to one skilled in the art and specific conditions are set forth in the Examples appended hereto.

In another aspect, the microorganism is inside, or on the surface of an animal. In an exemplary embodiment, the animal is a member selected from human, cattle, deer, reindeer, goat, honey bee, pig, sheep, horse, cow, bull, dog, guinea pig, gerbil, rabbit, cat, camel, yak, elephant, ostrich, otter, chicken, duck, goose, guinea fowl, pigeon, swan, and turkey. In another exemplary embodiment, the animal is a human.

In an exemplary embodiment, the microorganism is killed or its growth is inhibited through oral administration of the compound of the invention. In an exemplary embodiment, the microorganism is killed or its growth is inhibited through intravenous administration of the compound of the invention. In an exemplary embodiment, the microorganism is killed or its growth is inhibited through topical administration of the compound of the invention. In an exemplary embodiment, the microorganism is killed or its growth is inhibited through intraperitoneal administration of the compound of the invention. In an exemplary embodiment, the compound is administered in a topically effective amount. In an exemplary embodiment, the compound is administered in a cosmetically effective amount. In an exemplary embodiment, the pharmaceutical formulation is administered in an orally effective amount.

V. Methods of Treating and/or Preventing Disease

The compounds of the invention exhibit potency against microorganisms, such as protozoa, and therefore have the potential to achieve therapeutic efficacy in the animals described herein.

In another aspect, the invention provides a method of treating and/or preventing a disease. The method includes administering to the animal a therapeutically effective amount of the compound of the invention, sufficient to treat and/or prevent the disease. In an exemplary embodiment, the compound of the invention can be used in human or veterinary medical therapy, particularly in the treatment or prophylaxis of protozoa-associated disease. In an exemplary embodiment, the compound of the invention can be used in human or veterinary medical therapy, particularly in the treatment or prophylaxis of kinetoplastid-associated disease. In an exemplary embodiment, the disease is associated with a Trypanosoma. In an exemplary embodiment, the Trypanosoma is a member selected from T. avium, T. boissoni, T. brucei, T. carassii, T. cruzi, T. congolense, T equinum, T. equiperdum, T evansi, T. hosei, T. levisi, T. melophagium, T. parroti, T. percae, T rangeli, T. rotatorium, T. rugosae, T. sergenti, T. simiae, T sinipercae, T. suis, T. theileri, T. triglae and T. vivax. In an exemplary embodiment, the disease is associated with a Trypanosoma brucei. In an exemplary embodiment, the disease is associated with a member selected from Trypanosoma brucei brucei, Trypanosoma brucei rhodesiense and Trypanosoma brucei gambiense. In an exemplary embodiment, the disease is associated with Trypanosoma brucei rhodesiense. In an exemplary embodiment, the disease is associated with Trypanosoma brucei gambiense. In an exemplary embodiment, the disease is associated with Trypanosoma cruzi. In an exemplary embodiment, the disease is a trypanosomiasis. In an exemplary embodiment, the disease is a human trypanosomiasis. In an exemplary embodiment, the disease is an animal trypanosomiasis. In an exemplary embodiment, the disease is a member selected from nagana, surra, mal de caderas, murrina de caderas, dourine, cachexial fevers, Gambian horse sickness, baleri, kaodzera, tahaga, galziekte or galzietzke and peste-boba. In an exemplary embodiment, the disease is a member selected from Chagas disease (or Human American trypanosomiasis), nagana, surra, Covering sickness (or dourine) and sleeping sickness (or African sleeping sickness or Human African trypanosomiasis). In an exemplary embodiment, the disease is Chagas disease. In an exemplary embodiment, the disease is sleeping sickness (or African sleeping sickness). In an exemplary embodiment, the disease is acute phase sleeping sickness. In an exemplary embodiment, the disease is chronic phase sleeping sickness. In an exemplary embodiment, the disease is an acute phase of a trypanosomiasis. In an exemplary embodiment, the disease is a chronic phase of a trypanosomiasis. In an exemplary embodiment, the disease is the non-CNS form of a trypanosomiasis. In an exemplary embodiment, the disease is the CNS form of a trypanosomiasis. In an exemplary embodiment, the disease is the non-CNS form of sleeping sickness. In an exemplary embodiment, the disease is the CNS form of sleeping sickness. In an exemplary embodiment, the disease is early stage Human African trypanosomiasis. In an exemplary embodiment, the disease is late stage Human African trypanosomiasis. In another exemplary embodiment, the disease is associated with a member of the genus Leishmania. In another exemplary embodiment, the disease is associated with a member of Leishmania Viannia. In an exemplary embodiment, the disease is associated with a member selected from L. donovani, L. infantum, L. chagasi; L. mexicana, L. amazonensis, L. venezuelensis, L. tropica, L. major, L. aethiopica, L. (V.) braziliensis, L. (V.) guyanensis, L. (V.) panamensis, and L. (V.) peruviana. In an exemplary embodiment, the disease is associated with L. donovani. In an exemplary embodiment, the disease is associated with L. infantum. In an exemplary embodiment, the disease is leishmaniasis. In an exemplary embodiment, the disease is visceral leishmaniasis. In an exemplary embodiment, the disease is cutaneous leishmaniasis. In an exemplary embodiment, the disease is diffuse cutaneous leishmaniasis and/or mucocutaneous leishmaniasis. In another exemplary embodiment, the disease is associated with a member of the genus Plasmodium. In another exemplary embodiment, the disease is associated with a member selected from Plasmodium falciparum, Plasmodium vivax, Plasmodium ovale, Plasmodium vivax, Plasmodium malariae and Plasmodium knowlesi. In another exemplary embodiment, the disease is associated with a member selected from Plasmodium vivax, Plasmodium ovale, Plasmodium vivax and Plasmodium malariae. In another exemplary embodiment, the disease is associated with Plasmodium falciparum. In another exemplary embodiment, the disease is transmitted to the animal described herein by a mosquito infected with the protozoa. In another exemplary embodiment, the disease is transmitted to the animal described herein by an Anopheles mosquito containing the protozoa. In another exemplary embodiment, the disease is malaria. In another exemplary embodiment, the disease is cerebral malaria. In another exemplary embodiment, the disease is chronic malaria. In an exemplary embodiment, the compound is described herein, or a salt, prodrug, hydrate or solvate thereof, or a combination thereof. In an exemplary embodiment, the invention provides a compound described herein, or a salt, hydrate or solvate thereof. In an exemplary embodiment, the invention provides a compound described herein, or a prodrug thereof. In an exemplary embodiment, the invention provides a compound described herein, or a salt thereof. In another exemplary embodiment, the compound of the invention is a compound described herein, or a pharmaceutically acceptable salt thereof. In another exemplary embodiment, the compound is described by a formula listed herein, or a pharmaceutically acceptable salt thereof. In an exemplary embodiment, the compound is part of a pharmaceutical formulation described herein. In another exemplary embodiment, the contacting occurs under conditions which permit entry of the compound into the organism. In another exemplary embodiment, the compound of the invention is 7-(2-carboxyethyl)-1,3-dihydro-1-hydroxy-2,1-benzoxaborole, or a salt thereof. Such conditions are known to one skilled in the art and specific conditions are set forth in the Examples appended hereto.

In another exemplary embodiment, the animal is a member selected from human, cattle, deer, reindeer, goat, honey bee, pig, sheep, horse, cow, bull, dog, guinea pig, gerbil, rabbit, cat, camel, yak, elephant, ostrich, otter, chicken, duck, goose, guinea fowl, pigeon, swan, and turkey. In another exemplary embodiment, the animal is a human. In another exemplary embodiment, the animal is a mouse. In another exemplary embodiment, the animal is a member selected from a human, cattle, goat, pig, sheep, horse, cow, bull, dog, guinea pig, gerbil, rabbit, cat, chicken and turkey. In another exemplary embodiment, the animal is a human.

In an exemplary embodiment, the disease is treated through oral administration of the compound of the invention. In an exemplary embodiment, the disease is treated through intravenous administration of the compound of the invention. In an exemplary embodiment, the disease is treated through topical administration of the compound of the invention. In an exemplary embodiment, the disease is treated through intraperitoneal administration of the compound of the invention. In an exemplary embodiment, the compound is administered in a topically effective amount. In an exemplary embodiment, the compound is administered in a cosmetically effective amount. In an exemplary embodiment, the pharmaceutical formulation is administered in an orally effective amount.

In an exemplary embodiment, the disease is associated with an infection by a microorganism described herein. In an exemplary embodiment, the disease is associated with an infection by a protozoa described herein.

### VI. Pharmaceutical Formulations

In another aspect, the invention is a pharmaceutical formulation which includes: (a) a pharmaceutically acceptable excipient; and (b) a compound of the invention. In another aspect, the pharmaceutical formulation includes: (a) a pharmaceutically acceptable excipient; and (b) a compound according to a formula described herein. In another aspect, the pharmaceutical formulation includes: (a) a pharmaceutically acceptable excipient; and (b) a compound described herein, or a salt, prodrug, hydrate or solvate thereof, or a combination thereof. In another aspect, the pharmaceutical formulation includes: (a) a pharmaceutically acceptable excipient; and (b) a compound described herein, or a salt, hydrate or solvate thereof, or a combination thereof. In another aspect, the pharmaceutical formulation includes: (a) a pharmaceutically acceptable excipient; and (b) a compound described herein, or a salt, hydrate or solvate thereof. In another aspect, the pharmaceutical formulation includes: (a) a pharmaceutically acceptable excipient; and (b) a salt of a compound described herein. In an exemplary embodiment, the salt is a pharmaceutically acceptable salt. In another aspect, the pharmaceutical formulation includes: (a) a pharmaceutically acceptable excipient; and (b) a prodrug of a compound described herein. In another exemplary embodiment, the pharmaceutical formulation includes: (a) a pharmaceutically acceptable excipient; and (b) a compound described herein. In another exemplary embodiment, the pharmaceutical formulation includes: (a) a pharmaceutically acceptable excipient; and (b) 7-(2-carboxyethyl)-1,3-dihydro-1-hydroxy-2,1-benzoxaborole, or a salt thereof. In an exemplary embodiment, the pharmaceutical formulation is a unit dosage form. In an exemplary embodiment, the pharmaceutical formulation is a single unit dosage form.

The pharmaceutical formulations of the invention can take a variety of forms adapted to the chosen route of administration. Those skilled in the art will recognize various synthetic methodologies that may be employed to prepare non-toxic pharmaceutical formulations incorporating the compounds described herein. Those skilled in the art will recognize a wide variety of non-toxic pharmaceutically acceptable solvents that may be used to prepare solvates of the compounds of the invention, such as water, ethanol, propylene glycol, mineral oil, vegetable oil and dimethylsulfoxide (DMSO).

The pharmaceutical formulation of the invention may be administered orally, topically, intraperitoneally, parenterally, by inhalation or spray or rectally in unit dosage forms containing conventional non-toxic pharmaceutically acceptable carriers, adjuvants and vehicles. It is further understood that the best method of administration may be a combination of methods. Oral administration in the form of a pill, capsule, elixir, syrup, lozenge, troche, or the like is particularly preferred. The term parenteral as used herein includes subcutaneous injections, intradermal, intravascular (e.g., intravenous), intramuscular, spinal, intrathecal injection or like injection or infusion techniques. In an exemplary embodiment, the pharmaceutical formulation is administered orally. In an exemplary embodiment, the pharmaceutical formulation is administered intravenously. In an exemplary embodiment, the pharmaceutical formulation is administered in a topically effective dose. In an exemplary embodiment, the pharmaceutical formulation is administered in a cosmetically effective dose. In an exemplary embodiment, the pharmaceutical formulation is administered in an orally effective dose.

The pharmaceutical formulations containing compounds of the invention are preferably in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known in the art for the manufacture of pharmaceutical formulations, and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; and dispersing or wetting agents, which may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical formulations of the invention may also be in the form of oil-in-water emulsions and water-in-oil emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth; naturally-occurring phosphatides, for example soy bean, lecithin, and esters or partial esters derived from fatty acids and hexitol; anhydrides, for example sorbitan monooleate; and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical formulations may be in the form of a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The composition of the invention may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials are cocoa butter and polyethylene glycols.

Alternatively, the compositions can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

For administration to non-human animals, the composition containing the therapeutic compound may be added to the animal's feed or drinking water. Also, it will be convenient to formulate animal feed and drinking water products so that the animal takes in an appropriate quantity of the compound in its diet. It will further be convenient to present the compound in a composition as a premix for addition to the feed or drinking water. The composition can also added as a food or drink supplement for humans.

Dosage levels of the order of from about 5 mg to about 250 mg per kilogram of body weight per day and more preferably from about 25 mg to about 150 mg per kilogram of body weight per day, are useful in the treatment of the above-indicated conditions. The amount of active ingredient that may be combined with the carrier materials to produce a unit dosage form will vary depending upon the condition being treated and the particular mode of administration. Unit dosage forms will generally contain between from about 1 mg to about 500 mg of an active ingredient.

Frequency of dosage may also vary depending on the compound used and the particular disease treated. However, for treatment of most disorders, a dosage regimen of 4 times daily or less is preferred. It will be understood, however, that the specific dose level for any particular patient will depend upon a variety of factors including the activity of the specific compound employed, the age, body weight, general health, sex, diet, time of administration, route of administration and rate of excretion, drug combination and the severity of the particular disease undergoing therapy.

In an exemplary embodiment, the unit dosage form contains from about 1 mg to about 800 mg of a compound of the invention. In an exemplary embodiment, the unit dosage form contains from about 1 mg to about 500 mg of an active ingredient. In an exemplary embodiment, the unit dosage form contains from about 100 mg to about 800 mg of a compound of the invention. In an exemplary embodiment, the unit dosage form contains from about 200 mg to about 500 mg of a compound of the invention. In an exemplary embodiment, the unit dosage form contains from about 500 mg to about 800 mg of a compound of the invention. In an exemplary embodiment, the unit dosage form contains from about 1 mg to about 100 mg of a compound of the invention. In an exemplary embodiment, the unit dosage form contains from about 10 mg to about 100 mg of a compound of the invention. In an exemplary embodiment, the unit dosage form contains from about 50 mg to about 100 mg of a compound of the invention. In an exemplary embodiment, the unit dosage form contains from about 25 mg to about 75 mg of a compound of the invention. In an exemplary embodiment, the unit dosage form contains from about 40 mg to about 60 mg of a compound of the invention. In an exemplary embodiment, the unit dosage form contains from about 75 mg to about 200 mg of a compound of the invention. In an exemplary embodiment, the unit dosage form contains from about 1 mg to about 5 mg of a compound of the invention. In an exemplary embodiment, the unit dosage form contains from about 10 mg to about 25 mg of a compound of the invention. In an exemplary embodiment, the unit dosage form contains from about 50 mg to about 350 mg of a compound of the invention. In an exemplary embodiment, the unit dosage form contains from about 200 mg to about 400 mg of a compound of the invention.

In an exemplary embodiment, the daily dosage contains from about 1 mg to about 800 mg of a compound of the invention. In an exemplary embodiment, the daily dosage contains from about 1 mg to about 500 mg of an active ingredient. In an exemplary embodiment, the daily dosage contains from about 100 mg to about 800 mg of a compound of the invention. In an exemplary embodiment, the daily dosage contains from about 200 mg to about 500 mg of a compound of the invention. In an exemplary embodiment, the daily dosage contains from about 500 mg to about 800 mg of a compound of the invention. In an exemplary embodiment, the daily dosage contains from about 1 mg to about 100 mg of a compound of the invention. In an exemplary embodiment, the daily dosage contains from about 10 mg to about 100 mg of a compound of the invention. In an exemplary embodiment, the daily dosage contains from about 50 mg to about 100 mg of a compound of the invention. In an exemplary embodiment, the daily dosage contains from about 75 mg to about 200 mg of a compound of the invention. In an exemplary embodiment, the daily dosage contains from about 1 mg to about 5 mg of a compound of the invention. In an exemplary embodiment, the daily dosage contains from about 10 mg to about 25 mg of a compound of the invention. In an exemplary embodiment, the daily dosage contains from about 50 mg to about 350 mg of a compound of the invention. In an exemplary embodiment, the daily dosage contains from about 200 mg to about 400 mg of a compound of the invention.

Preferred compounds of the invention will have desirable pharmacological properties that include, but are not limited to, oral bioavailability, low toxicity, low serum protein binding and desirable in vitro and in vivo half-lives. Penetration of the blood brain barrier for compounds used to treat CNS disorders is necessary, while low brain levels of compounds used to treat peripheral disorders are often preferred.

Assays may be used to predict these desirable pharmacological properties. Assays used to predict bioavailability include transport across human intestinal cell monolayers, including Caco-2 cell monolayers. Toxicity to cultured hepatocycles may be used to predict compound toxicity. Penetration of the blood brain barrier of a compound in humans may be predicted from the brain levels of laboratory animals that receive the compound intravenously.

Serum protein binding may be predicted from albumin binding assays. Such assays are described in a review by Oravcova, et al. (Journal of Chromatography B (1996) volume 677, pages 1-27).

Compound half-life is inversely proportional to the frequency of dosage of a compound. In vitro half-lives of compounds may be predicted from assays of microsomal half-life as described by Kuhnz and Gieschen (Drug Metabolism and Disposition, (1998) volume 26, pages 1120-1127).

The amount of the composition required for use in treatment will vary not only with the particular compound selected but also with the route of administration, the nature of the condition being treated and the age and condition of the patient and will ultimately be at the discretion of the attendant physician or clinician.

### VI. a) Testing

Preferred compounds for use in the pharmaceutical formulations described herein will have certain pharmacological properties. Such properties include, but are not limited to, low toxicity, low serum protein binding and desirable in vitro and in vivo half-lives. Assays may be used to predict these desirable pharmacological properties. Assays used to predict bioavailability include transport across human intestinal cell monolayers, including Caco-2 cell monolayers. Serum protein binding may be predicted from albumin binding assays. Such assays are described in a review by Oravcova et al. (1996, J. Chromat. B677: 1-27). Compound half-life is inversely proportional to the frequency of dosage of a compound. In vitro half-lives of compounds may be predicted from assays of microsomal half-life as described by Kuhnz and Gleschen (Drug Metabolism and Disposition, (1998) volume 26, pages 1120-1127).

Toxicity and therapeutic efficacy of such compounds can be determined by standard pharmaceutical procedures in cell cultures or experimental animals, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds that exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage can vary within this range depending upon the unit dosage form employed and the route of administration utilized. The exact formulation, route of administration and dosage can be chosen by the individual physician in view of the patient's condition. (See, e.g. Fingl et al., 1975, in “The Pharmacological Basis of Therapeutics”, Ch. 1, p. 1).

### VI. b) Administration

For any compound used in the method of the invention, the therapeutically effective dose can be estimated initially from cell culture assays, as disclosed herein. For example, a dose can be formulated in animal models to achieve a circulating concentration range that includes the EC₅₀ (effective dose for 50% increase) as determined in cell culture, i.e., the concentration of the test compound which achieves a half-maximal inhibition of protozoa cell growth. Such information can be used to more accurately determine useful doses in humans.

In general, the compounds prepared by the methods, and from the intermediates, described herein will be administered in a therapeutically or cosmetically effective amount by any of the accepted modes of administration for agents that serve similar utilities. It will be understood, however, that the specific dose level for any particular patient will depend upon a variety of factors including the activity of the specific compound employed, the age, body weight, general health, sex, diet, time of administration, route of administration, and rate of excretion, drug combination, the severity of the particular disease undergoing therapy and the judgment of the prescribing physician. The drug can be administered from once or twice a day, or up to 3 or 4 times a day.

Dosage amount and interval can be adjusted individually to provide plasma levels of the active moiety that are sufficient to maintain protozoa cell growth inhibitory effects. Usual patient dosages for systemic administration range from 0.1 to 1000 mg/day, preferably, 1-500 mg/day, more preferably 10-200 mg/day, even more preferably 100-200 mg/day. Stated in terms of patient body surface areas, usual dosages range from 50-91 mg/m²/day.

The amount of the compound in a formulation can vary within the full range employed by those skilled in the art. Typically, the formulation will contain, on a weight percent (wt %) basis, from about 0.01-10 wt % of the drug based on the total formulation, with the balance being one or more suitable pharmaceutical excipients. Preferably, the compound is present at a level of about 0.1-3.0 wt %, more preferably, about 1.0 wt %.

Exemplary embodiments are summarized herein below.

In an exemplary embodiment, the invention is a compound having a structure according to the following formula:

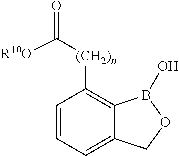

wherein n is 1 or 2 or 3 or 4 or 5, and R¹⁰ is H or C₁-C₆ alkyl, or a salt thereof.

In an exemplary embodiment, according to the above paragraph, the compound has a structure according to the following formula:

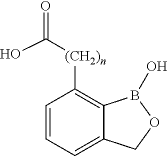

wherein n is 1 or 2 or 3 or 4 or 5.

In an exemplary embodiment, according to any of the above paragraphs, the compound is

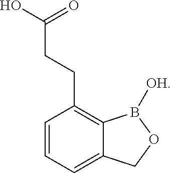

In an exemplary embodiment, the invention provides a combination comprising the compound according to any of the above paragraphs, together with at least one other therapeutically active agent.

In an exemplary embodiment, the invention provides a pharmaceutical formulation comprising: a) the compound according to any of the above paragraphs, or a salt thereof; and b) a pharmaceutically acceptable excipient.

In an exemplary embodiment, according to any of the above paragraphs, the pharmaceutical formulation is a unit dosage form.

In an exemplary embodiment, according to any of the above paragraphs, the salt of the compound according to any of the above paragraphs is a pharmaceutically acceptable salt.

In an exemplary embodiment, the invention provides a method of killing and/or preventing the growth of a protozoa, comprising: contacting the protozoa with an effective amount of the compound of the invention, thereby killing and/or preventing the growth of the protozoa.

In an exemplary embodiment, according to any of the above paragraphs, the compound has a structure described herein.

In an exemplary embodiment, according to any of the above paragraphs, the protozoa is a member of the trypanosome genus.

In an exemplary embodiment, according to any of the above paragraphs, the protozoa is a member of the leishmania genus.

In an exemplary embodiment, according to any of the above paragraphs, the protozoa is a member of the plasmodium genus.

In an exemplary embodiment, according to any of the above paragraphs, the protozoa is Trypanosoma brucei.

In an exemplary embodiment, according to any of the above paragraphs, the Trypanosoma brucei is a member selected from Trypanosoma brucei brucei, Trypanosoma brucei gambiense and Trypanosoma brucei rhodesiense.

In an exemplary embodiment, according to any of the above paragraphs, the protozoa is a member selected from Leishmania donovani, Leishmania infantum, Leishmania chagasi, Leishmania mexicana, Leishmania amazonensis, Leishmania venezuelensis, Leishmania tropica, Leishmania major, Leishmania aethiopica.

In an exemplary embodiment, according to any of the above paragraphs, the protozoa is Leishmania donovani.

In an exemplary embodiment, according to any of the above paragraphs, the protozoa is a member selected from Plasmodium falciparum, Plasmodium vivax, Plasmodium ovale, Plasmodium vivax, Plasmodium malariae and Plasmodium knowlesi.

In another exemplary embodiment, according to any of the above paragraphs, the protozoa is Plasmodium falciparum.

In an exemplary embodiment, the invention provides a method of treating and/or preventing a disease in an animal, comprising: administering to the animal a therapeutically effective amount of the compound of the invention, thereby treating and/or preventing the disease.

In an exemplary embodiment, according to any of the above paragraphs, the compound has a structure described herein.

In an exemplary embodiment, according to any of the above paragraphs, the disease is African sleeping sickness.

In an exemplary embodiment, according to any of the above paragraphs, the disease is leishmaniasis.

In an exemplary embodiment, according to any of the above paragraphs, the leishmaniasis is a member selected from visceral leishmaniasis, cutaneous leishmaniasis, diffuse cutaneous leishmaniasis and mucocutaneous leishmaniasis.

In an exemplary embodiment, according to any of the above paragraphs, the leishmaniasis is visceral leishmaniasis.

In an exemplary embodiment, according to any of the above paragraphs, the leishmaniasis is cutaneous leishmaniasis.

In an exemplary embodiment, according to any of the above paragraphs, the disease is malaria.

In an exemplary embodiment, according to any of the above paragraphs, the disease is cerebral malaria.

In an exemplary embodiment, according to any of the above paragraphs, the animal is a human.

In an exemplary embodiment, according to any of the above paragraphs, the invention is a use of a compound of the invention or a combination of the invention in the manufacture of a medicament for the treatment and/or prophylaxis of protozoal infection.

The invention is further illustrated by the Examples that follow. The Examples are not intended to define or limit the scope of the invention.

### EXAMPLES

The following Examples illustrate the synthesis of representative compounds used in the invention and the following Reference Examples illustrate the synthesis of intermediates in their preparation. These examples are not intended, nor are they to be construed, as limiting the scope of the invention. It will be clear that the invention may be practiced otherwise than as particularly described herein. Numerous modifications and variations of the invention are possible in view of the teachings herein and, therefore, are within the scope of the invention.

All temperatures are given in degrees Centigrade. Room temperature means 20 to 25° C. Reagents were purchased from commercial sources or prepared following standard literature procedures. Unless otherwise noted, reactions were carried out under a positive pressure of nitrogen. Reaction vessels were sealed with either rubber septa or Teflon screw caps. Nitrogen was introduced through Tygon tubing, fitted with a large bore syringe needle. Concentration under vacuum refers to the removal of solvent on a Büchi Rotary Evaporator.

Analytical HPLC was performed using a Supelco discovery C₁₈ 15 cm×4.6 mm/5 μm column coupled with an Agilent 1050 series VWD UV detector at 210 nm. Conditions: Solvent A: H₂O/1% acetonitrile/0.1% HCO₂H; Solvent B: methanol.

Proton magnetic resonance (¹H NMR) spectra were recorded on a Varian INOVA NMR spectrometer [400 MHz (¹H) or 500 MHz (¹H)]. All spectra were determined in the solvents indicated. Although chemical shifts are reported in ppm downfield of tetramethylsilane, they are referenced to the residual proton peak of the respective solvent peak for ¹H NMR. Interproton coupling constants are reported in Hertz (Hz).

LCMS spectra were obtained using a ThermoFinnigan AQA MS ESI instrument utilizing a Phenomenex Aqua 5 micron C₁₈ 125 Å 50×4.60 mm column. The spray setting for the MS probe was at 350 μL/min with a cone voltage at 25 mV and a probe temperature at 450° C. The spectra were recorded using ELS and UV (254 nm) detection. Alternatively, LCMS spectra were obtained using an Agilent 1200SL HPLC equipped with a 6130 mass spectrometer operating with electrospray ionization.

Silica gel chromatography was carried out on either a Teledyne ISCO CombiFlash Companion or Companion Rf Flash Chromatography System with a variable flow rate from 5-100 mL/min. The columns used were Teledyne ISCO RediSep Disposable Flash Columns (4, 12, 40, 80, or 120 g prepacked silica gel), which were run with a maximum capacity of 1 g crude sample per 10 g silica gel. Samples were preloaded on Celite in Analogix Sample Loading Cartridges with fits (1/in, 1/out). The eluent was 0-100% EtOAc in heptane or 0-10% MeOH in CH₂Cl₂ as a linear gradient over the length of the run (14-20 minutes). Peaks were detected by variable wavelength UV absorption (200-360 nm). The resulting fractions were analyzed, combined as appropriate, and evaporated under reduced pressure to provide purified material.

HPLC purification was performed using a 50 mm Varian Dynamax HPLC 21.4 mm Microsorb Guard-8 C₁₈ column, Dyonex Chromeleon operating system coupled with a Varian Prostar 320 UV-vis detector (254 nm) and a Sedex55 ELS detector. Conditions: Solvent A: H₂O/1% acetonitrile/0.1% HCO₂H; Solvent B: MeOH. The appropriate solvent gradient for purification was determined based on the results of analytical HPLC experiments. The resulting fractions were analyzed, combined as appropriate, and evaporated under reduced pressure to provide purified material.

The following experimental sections illustrate procedures for the preparation of intermediates and methods for the preparation of products according to this invention. It should be evident to those skilled in the art that appropriate substitution of both the materials and methods disclosed herein will produce the examples illustrated below and those encompassed by the scope of the invention.

All solvents used were commercially available and were used without further purification. Reactions were typically run using anhydrous solvents under an inert atmosphere of N₂.

Compounds are named using the AutoNom 2000 add-on for MDL ISIS™ Draw 2.5 SP2 or their catalogue name if commercially available.

Starting materials used were either available from commercial sources or prepared according to literature procedures and had experimental data in accordance with those reported. 6-aminobenzo[c][1,2]oxaborol-1(3H)-ol (C50), for example, can be synthesized according to the methods described in U.S. Pat. Pubs. US20060234981 and US20070155699.

### Example 1

### 1 7-Formyl-1-hydroxy-1,3-dihydro-2,1-benzoxaborole

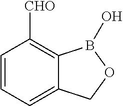

### 2-Bromo-1,3-benzenedicarboxylic acid

To a solution of commercially available 2,6-dimethylbromobenzene (60 g, 324.3 mmol) in t-BuOH (250 mL) and H₂O (250 mL) was added KMnO₄ (128 g, 0.81 mol) in portions while stirring at room temperature. The mixture was stirred at 70° C. for 2 h before it was cooled to room temperature. A second batch of KMnO₄ (128 g, 0.81 mol) was added as before. After stirring at 70° C. for 10 h, the hot reaction mixture was filtered and the residue was washed with water (3×300 mL). After concentration to 300 mL, the filtrate was acidified in ice-bath to pH 2 with conc. HCl to get white precipitate. After extraction with EtOAc (4×500 mL), the organic phase was dried with Na₂SO₄ and concentrated in vacuo to obtain 70.2 g of 2-bromo-1,3-benzenedicarboxylic acid (88.3%). ¹H NMR (400 MHz, DMSO-d₆): δ 13.58 (s, 2H), 7.68 (m, 2H), 7.50 (q, J₁=8 Hz, J₂=7.2 Hz, 4H); ¹³C NMR (100 MHz, DMSO-d₆): δ 168.1, 137.1, 131.1, 128.2, 116.6; HRMS-ES: C₈H₅BrO₄ calcd 243.9371. Found 243.9372.

### 2-Bromo-isophthalic acid dimethyl ester

2-Bromo-1,3-benzenedicarboxylic acid (44.20 g, 180.4 mmol) in SOCl₂ (300 mL) was gradually heated to 100° C. during a period of 5 h and stirred at 100° C. for another 4 h. After SOCl₂ was evaporated in vacuo and the flask was cooled to 0° C., methanol (200 mL) and triethylamine (100 mL) were added slowly while stirring. The reaction mixture was stirred at room temperature for 2 h and was concentrated in vacuo. The residue was extracted with EtOAc, dried over MgSO₄ and concentrated in vacuo to obtain 47.05 g of the title compound (95.5%). ¹H NMR (400 MHz, CDCl₃): δ 7.70 (m, 2H), 7.40 (t, 1H), 3.94 (s, 6H); ¹³C NMR (100 MHz, CDCl₃): δ 166.0, 134.8, 132.0, 126.9, 117.9, 52.5; HRMS-ES: C₁₀H₉BrO₄ calcd 241.9684. Found 241.9683.

### 2,6-Bis(hydroxymethyl) bromobenzene

To a solution of 2-bromo-isophthalic acid dimethyl ester (30.50 g, 112 mmol) in ethyl ether (300 mL) was added LiBH₄ (5.42 g, 245.8 mmol) in THF (100 mL) slowly at 0° C. The reaction mixture was stirred at room temperature overnight, quenched with HCl to pH 6-7, and extracted with ethyl acetate to obtain 24.51 g of 2,6-bis(hydroxymethyl) bromobenzene (100%).

Alternative method: 2-bromo-isophthalic acid dimethyl ester (13.65 g, 50.0 mmol) was dissolved in 250 mL 1,4-Dioxane-H₂O (3:2, 250 mL) and cooled to 0° C. To this mixture was added NaBH₄ (18.90 g, 0.50 mol) and stirred at room temperature for 2 d before it was quenched with 6 M HCl in ice-bath, extracted with ethyl acetate, washed with saturated NaHCO₃ and brine, dried over Na₂SO₄, and concentrated in vacuo to obtain 8.50 g of 2,6-bis(hydroxymethyl) bromobenzene (78.1%). ¹H NMR (400 MHz, DMSO-d₆): δ 7.39 (m, 3H), 5.39 (t, J=5.6 Hz, 2H), 4.52 (d, J=5.6 Hz, 4H); ¹³C NMR (100 MHz, DMSO-d₆): δ 141.0, 127.0, 126.3, 120.4, 62.9; HRMS-ES: C₈H₉BrO₂ calcd 215.9786. Found 215.9783.

### 2-Bromo-benzene-1,3-dicarbaldehyde

A mixture of 2,6-bis(hydroxymethyl) bromobenzene (12.0 g, 55.3 mmol), PCC (35.7 g, 165.9 mmol) and Celite (53.6 g) in CH₂Cl₂ (500 mL) was stirred at room temperature overnight. The reaction mixture was filtered through Celite and silica gel pad and the filtrate was evaporated in vacuo to obtain 10.90 g of 2-bromo-benzene-1,3-dicarbaldehyde (92.4%). ¹H NMR (400 MHz, DMSO-d₆): δ 10.37 (s, 2H), 8.08 (m, 2H) and 7.71 (m, 1H).

### 2-Bromo-3-[1,3]-dioxolan-2-yl-benzaldehyde

2-Bromo-benzene-1,3-dicarbaldehyde (7.1 g, 33.3 mmol) and p-toluenesulfonic acid monohydrate (130 mg, 0.68 mmol) were dissolved in toluene (250 mL) and heated to reflux. Ethylene glycol (1.83 mL, 33.3 mmol) was added dropwise and water was removed by azeotropic evaporation with toluene for 2 h. The reaction mixture was cooled to room temperature, washed with saturated NaHCO₃ (100 mL), and extracted with ethyl acetate. The organic phase was washed with brine, dried over Na₂SO₄, evaporated in vacuo, and purified by column chromatography to obtain 5.35 g of 2-bromo-3-[1,3]dioxolan-2-yl-benzaldehyde (62.5%). ¹H NMR (400 MHz, DMSO-d₆): δ 10.33 (s, 1H), 7.86 (m, 2H), 7.60 (m, 1H), 6.10 (s, 1H) and 4.06 (m, 4H).

### 2-(2-Bromo-3-methoxymethoxymethyl-phenyl)-[1,3]-dioxolane

To a solution of 2-bromo-3-[1,3]dioxolan-2-yl-benzaldehyde (13.10 g, 51.0 mmol) in methanol (500 mL) at 0° C. was added NaBH₄ (3.85 g, 101.9 mmol). The reaction mixture was stirred at room temperature for 1 h before H₂O (100 mL) was added, and the mixture was concentrated in vacuo and extracted with ethyl acetate. The organic phase was washed with brine, dried over Na₂SO₄, and evaporated in vacuo to obtain 13.23 g of compound a (100%). ¹H NMR (400 MHz, DMSO-d₆): δ 7.55 (m, 1H), 7.42 (m, 2H), 6.00 (s, 1H), 5.47 (t, 1H), 4.52 (d, 2H) and 4.01 (m, 4H). To a solution of compound a (5.0 g, 19.31 mmol) and DIPEA (7.0 mL, 40.0 mmol) in CH₂Cl₂ (120 ml) was added MOMCl (2.85 mL, 38.61 mmol) and the mixture was stirred at room temperature for 7 h before the solvent was evaporated in vacuo. The residue was purified by column chromatography to obtain 5.45 g of 2-(2-bromo-3-methoxymethoxymethyl-phenyl)-[1,3]dioxolane (93.2%). ¹H NMR (300 MHz, CDCl₃): δ 7.53 (m, 2H), 7.35 (m, 1H), 6.17 (s, 1H), 4.77 (s, 2H), 4.71 (s, 2H), 4.11 (m, 4H) and 3.43 (s, 3H).

### 7-Formyl-1-hydroxy-1,3-dihydro-2,1-benzoxaborole

To a solution of 2-(2-bromo-3-methoxymethoxymethyl-phenyl)-[1,3]dioxolane (5.45 g, 17.97 mmol) in THF (120 mL) at −78° C. was added n-BuLi (1.6 M in hexane, 12.43 mL, 19.79 mmol) dropwise. The reaction mixture was stirred at −78° C. for 20 min and triisopropyl borate (4.62 mL, 19.79 mmol) was added. The reaction was allowed to warm to room temperature and stirred overnight before it was quenched with 6 M HCl (40 mL), concentrated in vacuo, extracted with ethyl acetate, washed with brine, dried with Na₂SO₄, evaporated in vacuo, and purified by re-crystallization (hexane: EtOAc=3:1) to obtain 2.23 g of 7-formyl-1-hydroxy-1,3-dihydro-2,1-benzoxaborole (76.7%). ¹H NMR (400 MHz, DMSO-d₆): δ10.39 (s, 1H), 9.22 (s, 1H), 7.87 (m, 1H), 7.72 (m, 2H), 5.13 (s, 2H); ¹³C NMR (100 MHz, CD₃OD): δ 142.9, 132.4, 132.3, 132.0, 125.3, 122.5, 104.1, 72.1.

### 2 7-Cyano-1,3-dihydro-1-hydroxy-2,1-benzoxaborole

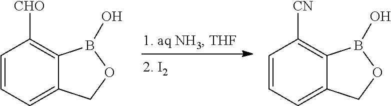

To a solution of 7-formyl-1-hydroxy-1,3-dihydro-2,1-benzoxaborole (0.6 g, 3.7 mmol) in THF (5 ml) was added under stirring concentrated ammonia (50 ml). After stirring for 5 minutes iodine (1.03 g, 4.1 mmol) was added in portions. The reaction was stirred at room temperature for 2 hours before 5% Na₂S₂O₃ (25 mL) was added. After stirring for another 2 hours the mixture was acidified to pH=3 with concentrated HCl, then poured into water (100 mL) and extracted with dichloromethane. The combined organic phase was dried over anhydrous MgSO₄ and evaporated to give the title compound (0.56 g, 96% yield). ¹H NMR (400 MHz, DMSO-d₆): δ 9.47 (s, 1H), 7.81 (d, J=7.6 Hz, 1H), 7.76 (d, J=7.6 Hz, 1H), 7.68 (d, J=7.6 Hz, 1H) and 5.07 (s, 2H) ppm. Mp 151-152° C.

### 3 7-Carboxyl-1-hydroxy-1,3-dihydro-2,1-benzoxaborole

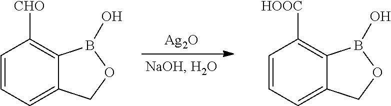

The preparation of silver oxide: silver nitrate (629.6 mg, 3.70 mmol, 2.0 eq) in water (4.5 mL) was added to a solution of sodium hydroxide (0.75 g) in water (4.5 mL). Continuous shaking during the addition ensures complete reaction. A brown semisolid mixture was obtained.

To this mixture, which was cooled to 0° C., was added 7-formyl-1-hydroxy-1,3-dihydro-2,1-benzoxaborole (300 mg, 1.85 mmol) in small portions with stirring for 30 minutes. The mixture was acidified to pH 2 and extracted by ethyl acetate. The organic phase was dried over anhydrous Na₂SO₄ and evaporated to give the crude product (290 mg). The crude product was purified by recrystallization to give the title compound (160 mg, 48.6% yield). ¹H NMR (400 MHz, DMSO-d₆): δ 8.86 (s, 1H), 7.99 (m, 1H), 7.72 (m, 1H), 7.65 (m, 1H) and 5.10 (s, 2H) ppm. Mp 191-193° C.

### 4 7-Methoxycarbonyl-1-hydroxy-1,3-dihydro-2,1-benzoxaborole

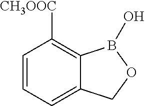

This compound can be prepared by contacting 7-carboxyl-1-hydroxy-1,3-dihydro-2,1-benzoxaborole with methanol and catalytic sulfuric acid, and refluxing the mixture for about 2 hours. ¹H NMR (300 MHz, DMSO-d₆): δ 8.44 (s, 1H), 7.96 (d, 1H), 7.71 (d, 1H), 7.66 (t, 1H), 5.08 (s, 2H) 3.92 (s, 3H) ppm. Mass: m/z=193 (M+1, ESI+).

### 5 2-(1-Hydroxy-1,3-dihydrobenzo[c][1,2]oxaborol-7-yl)acetic acid

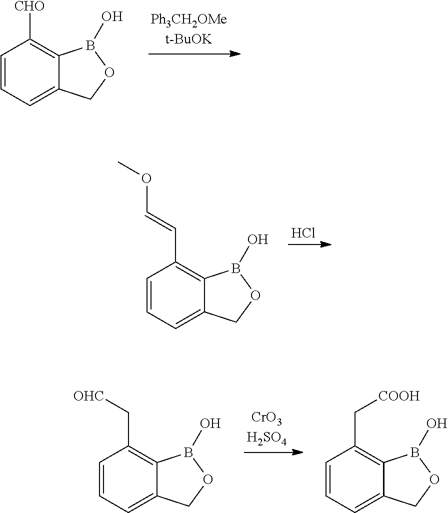

The title compound may be prepared by using the scheme above and following the similar procedures described for compound 64.

### 6 7-(2-Carboxyethyl)-1,3-dihydro-1-hydroxy-2,1-benzoxaborole

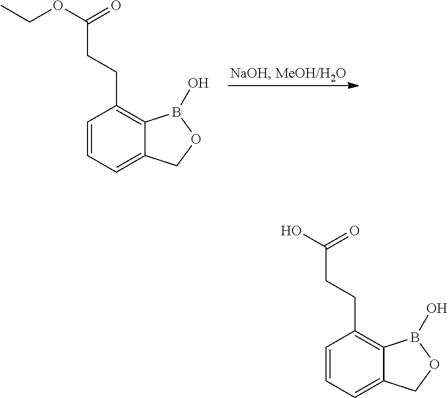

To a solution of [3-(1,3-dihydro-1-hydroxy-2,1-benzoxaborol-7-yl)]propionic acid ethyl ester (320 mg, 1.38 mmol) in 20 ml methanol was added 1M NaOH (10 ml). The mixture was heated to reflux for 1 hour. After it was cooled to room temperature the mixture was acidified to pH=2 and extracted with ethyl acetate. The organic phase was dried over anhydrous Na₂SO₄ and evaporated to give the title compound (240 mg, 85.7% yield). ¹H NMR (400 MHz, DMSO-d₆): δ 12.07 (s, 1H), 8.98 (s, 1H), 7.36 (m, 1H), 7.21 (m, 1H), 7.14 (m, 1H), 4.96 (s, 2H), 2.99 (t, J=8.2 Hz, 2H) and 2.54 (t, J=8.0 Hz, 2H) ppm. Mp 142-144° C.

### 7 Methyl 3-(1-hydroxy-1,3-dihydrobenzo[c][1,2]oxaborol-7-yl)propanoate

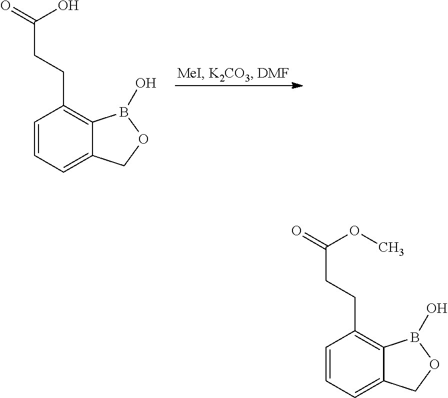

To a solution of 3-(1-hydroxy-1,3-dihydrobenzo[c][1,2]oxaborol-7-yl)propanoic acid (300 mg, 1.45 mmol, 1.0 eq) in DMF (7 mL) was added K₂CO₃ (501 mg, 3.625 mmol, 2.5 eq). The reaction was stirred at room temperature for 10 min. Iodomethane (907 uL, 14.56 mmol, 10 eq) was added. The reaction was stirred at room temperature for 1 h. The reaction was quenched with 1N HCl and extracted with t-butyl methyl ether (TBME). The organic phase was washed with saturated NaHCO₃, then brine, dried over anhydrous Na₂SO₄ and filtered. The residue after rotary evaporation was purified by column chromatography to give the desired product as white solid (220 mg, 69% yield). ¹H NMR (300 MHz, DMSO-d₆): δ 9.00 (s, 1H), 7.40 (t, J=7.5 Hz, 1H), 7.21 (d, J=3 Hz, 1H), 7.15 (d, J=4.5 Hz, 1H), 4.96 (s, 2H), 3.81 (s, 3H), 3.00 (t, J=7.5, 2H), 2.65 (t, J=4.5, 2H) ppm. Mass: m/z=221.1 (M+1, ESI+).

### 8 [3-(1,3-Dihydro-1-hydroxy-2,1-benzoxaborol-7-yl)]propionic acid ethyl ester

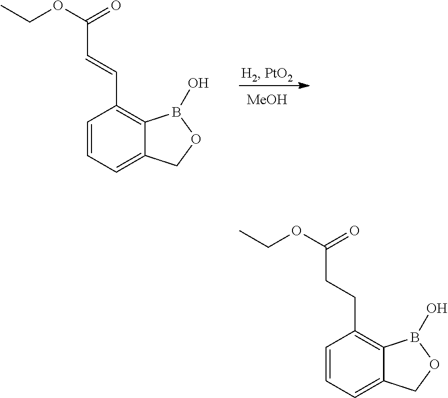

To a solution of (E)-[3-(1,3-dihydro-1-hydroxy-2,1-benzoxaborol-7-yl)]acrylic acid ethyl ester (1.20 g, 5.17 mmol) in methanol (10 mL) was added platinum dioxide trihydrate (73 mg, 0.26 mmol, 0.05 eq). The reaction mixture was vacuumed and backfilled hydrogen for 3 times, then stirred overnight at room temperature. The mixture was filtered and the filtrate was evaporated to give the title compound (1.16 g, 96% yield). ¹H NMR (400 MHz, CD₃OD): δ 7.34 (t, J=7.6 Hz, 1H), 7.18 (d, J=7.6 Hz, 1H), 7.11 (d, J=7.2 Hz, 1H), 5.02 (s, 2H), 4.06 (q, J=7.2 Hz, 2H), 3.05 (t, J=7.6 Hz, 2H), 2.61 (t, J=7.6 Hz, 2H) and 1.19 (t, J=7.2 Hz, 3H) ppm. Mp 93-95° C.

### 9 2,2-Difluoro-3-(1-hydroxy-1,3-dihydrobenzo[c][1,2]oxaborol-7-yl)propanoic acid

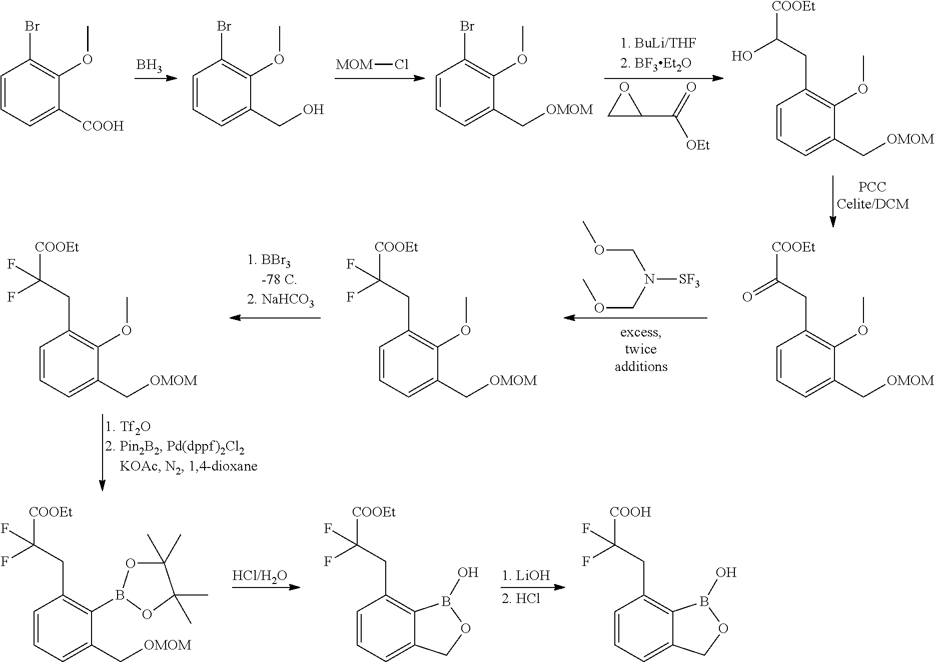

The title compound may be prepared from commercially available 3-bromo-2-methoxybenzoic acid by using the scheme above.

### 10 3,3-Difluoro-3-(1-hydroxy-1,3-dihydrobenzo[c][1,2]oxaborol-7-yl)propanoic acid

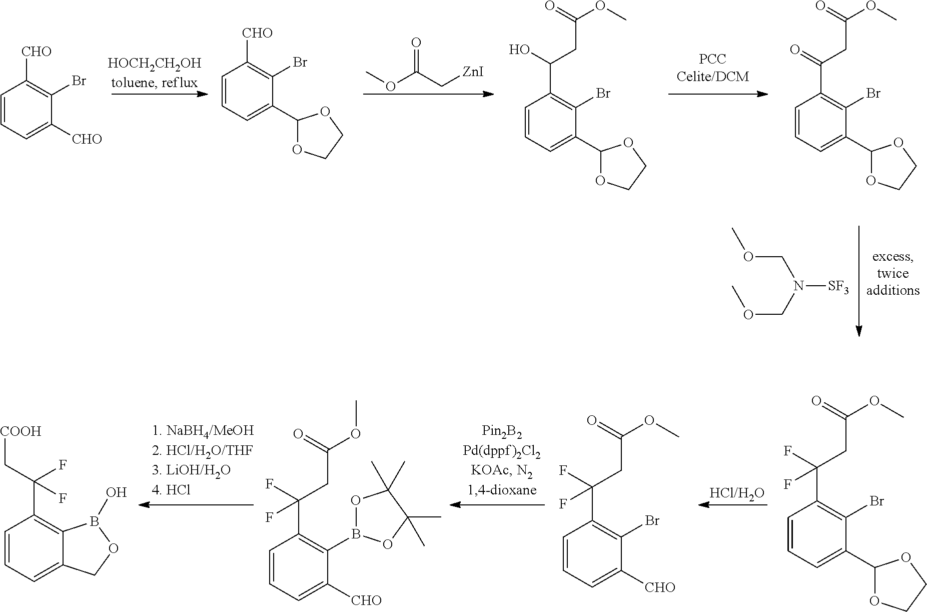

The title compound may be prepared from commercially available 2,6-diformylphenylbromide by using the scheme above.

### 11 2-Amino-3-(1-hydroxy-1,3-dihydrobenzo[c][1,2]oxaborol-7-yl)propanoic acid

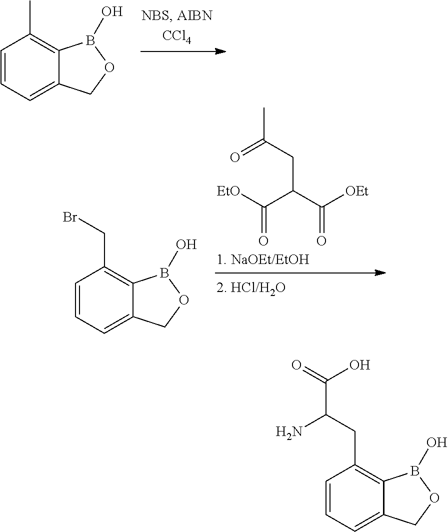

The title compound may be prepared from 7-methylbenzo[c][1,2]oxaborol-1(3H)-ol by using the scheme above. Method for the synthesis of 7-methylbenzo[c][1,2]oxaborol-1(3H)-ol has been reported in WO2009111676 A2 and WO2007095638 A2, WO2007078340 A2 and US2007155699 A1.

### 12 2-Hydroxy-3-(1-hydroxy-1,3-dihydrobenzo[c][1,2]oxaborol-7-yl)propanoic acid

The title compound may be prepared according to the following scheme.

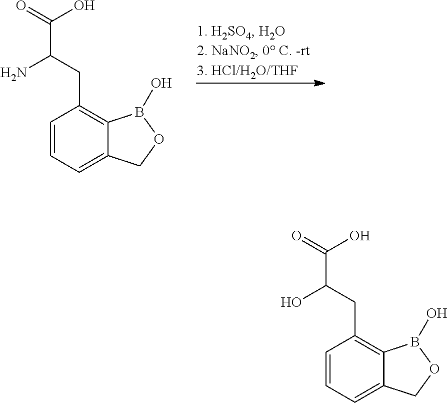

### 13 4-(1-Hydroxy-1,3-dihydrobenzo[c][1,2]oxaborol-7-yl)butanoic acid

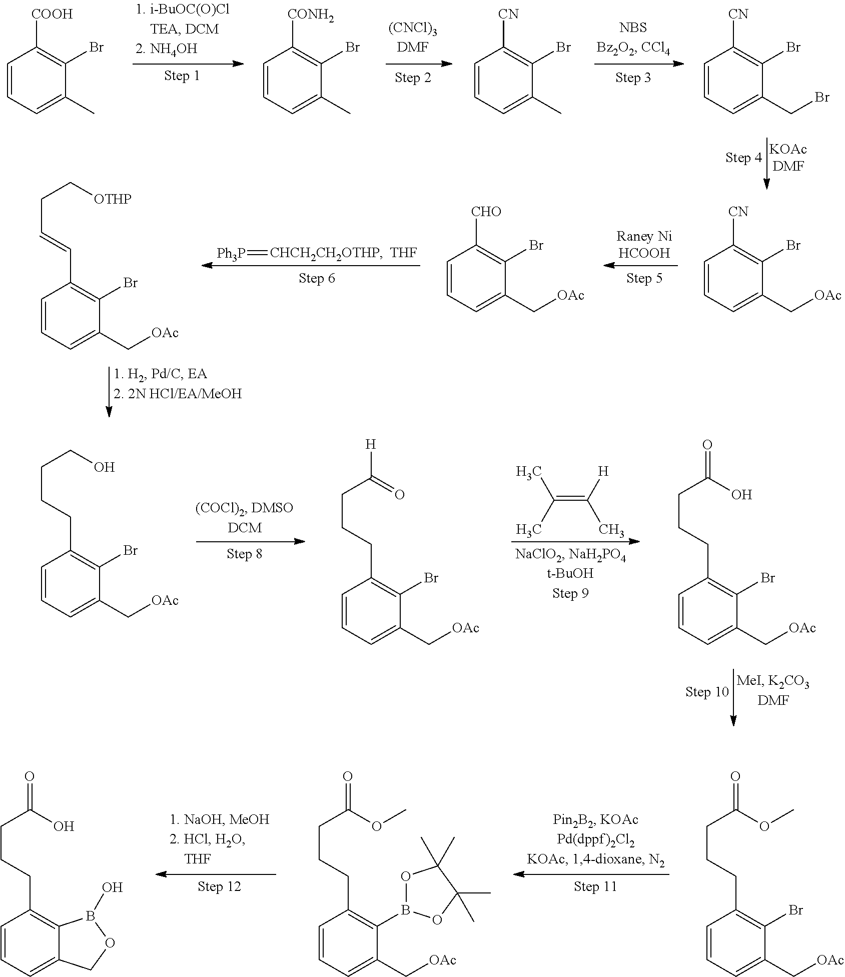

### Step 1: Preparation of 2-bromo-3-methylbenzamide

To a solution of 2-bromo-3-methylbenzoic acid (20 g, 92 mmol, 1.0 eq) in dichloromethane (125 mL) was added TEA (14.7 mL, 100 mmol, 1.1 eq). Isobutyl chloroformate (12.5 mL, 100 mmol, 1.1 eq) in dichloromethane (25 mL) was added dropwise to the solution at ice-water for 10 min. And then ammonia (40.37 mL, 650 mmol, 7.0 eq) was added dropwise at ice-water for 2 min. The reaction mixture was added water (25 mL) and cooled to the r.t. and filtered. Solid was washed with water (2×100 mL), and 0.5N HCl (2×25 mL). The solid was dried in vacuum until constant weight to give solid (14.1 g, yield 71%); Purity by ¹H-NMR: 90%. This intermediate was used for the next reaction without further purification.

### Step 2: Preparation of 2-bromo-3-methylbenzonitrile

To a solution of 2-bromo-3-methylbenzamide (54.5 g, 254 mmol, 1.0 eq) in DMF (327 mL) cooled with an ice-water bath was added cyanuric chloride (70.4 g, 382 mmol, 1.5 eq). The reaction was stirred at r.t. overnight. The reaction mixture was quenched with water and extracted with ethyl acetate. The organic phase was washed with 0.5N HCl, brine, dried over anhydrous sodium sulfate and concentrated to give 2-bromo-3-methylbenzonitrile (47.4 g, yield 94.9%); Purity by 1H-NMR: 90%. This intermediate was used for the next reaction without further purification.

### Step 3: Preparation of 2-bromo-3-(bromomethyl)benzonitrile

To a solution of 2-bromo-3-methylbenzonitrile (12 g, 51 mmol, 1.0 eq) in tetrachloromethane (127.5 mL) was added N-bromosuccinimide (10 g, 56.1 mmol, 1.1 eq) and Bz₂O₂ (0.075 g, 0.31 mmol, 0.006 eq) at r.t. The reaction flask was vacuumed and backfilled by nitrogen. The reaction was stirred at reflux overnight. The reaction was cooled and stirred at r.t. Insoluble matter was removed by filtration. The filtrate was cooled with ice-water, and then filtered to give 2-bromo-3-(bromomethyl)benzonitrile (7.6 g, yield 54%): Purity by ¹H-NMR: 90%.

### Step 4: Preparation of 2-bromo-3-cyanobenzyl acetate

To a solution of 2-bromo-3-(bromomethyl)benzonitrile (30 g, 109 mmol, 1 eq) in DMF (253 mL) was added KOAc (13 g, 131 mmol, 1.2 eq). The reaction was stirred at 82° C. for 1 h. The reaction was quenched with water and extracted with ethyl acetate. The organic phase was washed with 0.5N HCl, brine, and dried over anhydrous sodium sulfate. The organic phase was evaporated to give the crude residue that was stirred with petroleum ether (PE) and filtered to give 2-bromo-3-cyanobenzyl acetate (24.2 g, yield 87.4%); Purity by ¹H-NMR: 90%.

### Step 5: Preparation of 2-bromo-3-formylbenzyl acetate

To Raney Ni (0.533 g, 9 mmol, 2.3 eq) in formic acid (10 mL) and water (2 mL) was added 2-bromo-3-cyanobenzyl acetate (1 g, 3.9 mmol, 1.0 eq) at r.t. After addition, the reaction was stirred at 100° C. for 1 h. The reaction was then cooled to r.t. Insoluble material was removed by filtration. The filtrate was concentrated in vacuum to give a solid residue that was purified by column chromatography (EA:PE=1:4) to give 2-bromo-3-formylbenzyl acetate as a solid (600 mg, yield 60%): Purity by 1H-NMR: 90%.

### Step 6: Preparation of (E)-2-bromo-3-(4-(tetrahydro-2H-pyran-2-yloxy)but-1-enyl)benzyl acetate

A mixture of Ph₃P—CH₂CH₂CH₂OTHP bromide (3.77 g, 7.78 mmol, 2.0 eq) in THF (19.5 mL) was treated n-BuLi (2.644 mL, 6.61 mmol, 2.5M/hexanes) at −78° C., then stirred for 1 h. 2-Bromo-3-formylbenzyl acetate (1 g, 3.89 mmol, 1.0 eq) was then added, followed by removal of the cooling bath. After 20 h, the reaction mixture was diluted with ethyl acetate, and then washed with water, brine, dried over anhydrous sodium sulfate and evaporated. The crude was purified by column chromatography (EA/DCM=5%) to give (E)-2-bromo-3-(4-(tetrahydro-2H-pyran-2-yloxy)but-1-enyl)benzyl acetate (1.25 g, yield 87%); Purity by ¹H-NMR: 90%.

### Step 7: Preparation of 2-bromo-3-(4-hydroxybutyl)benzyl acetate

To a solution of (E)-2-bromo-3-(4-(tetrahydro-2H-pyran-2-yloxy)but-1-enyl)benzyl acetate (3.1 g, 8.09 mmol, 1 eq) in ethyl acetate (40 mL) was added Pd/C (310 mg, 10%). The reaction flask was vacuumed and backfilled with hydrogen for 3 times. The reaction was stirred at rt for 1 h. The reaction was filtered and evaporated. The residue was dissolved in ethyl acetate (8 mL), methanol (15 mL) and 2N HCl (8 ml), and then stirred at 40° C. for 0.5 h. The reaction was extracted with ethyl acetate. The organic phase was washed with brine, dried over anhydrous sodium sulfate and evaporated to give a crude residue. The residue was purified by column chromatography (EA/PE=30%) to give 2-bromo-3-(4-hydroxybutyl)benzyl acetate (1.54 g, yield 63.4%); Purity by ¹H NMR: 90%.

### Step 8: Preparation of 2-bromo-3-(4-oxobutyl)benzyl acetate

To a stirred solution of oxalyl chloride (480 ul, 5.6 mmol, 1.4 eq) in DCM (28 mL) was added DMSO (513 uL, 7.2 mmol, 1.8 eq) dropwise at −78° C. After gas evolution was subsided, 2-bromo-3-(4-hydroxybutyl)benzyl acetate (1.2 g, 4 mmol, 1.0 eq) in DCM (5 mL) was added. After 15 min, the white suspension was treated dropwise with TEA (2.8 ml, 20 mmol, 5.0 eq). After addition was completed, the cooling bath was removed and stirring was continued for 2 h. The reaction was diluted with DCM and then washed with water, brine, dried over anhydrous sodium sulfate, and evaporated to give a crude residue. The residue was purified by column chromatography (EA/PE=20%) to give 2-bromo-3-(4-oxobutyl)benzyl acetate (0.981 mg, yield 82%); Purity by ¹H NMR: 90%.

### Step 9: Preparation of 4-(3-(acetoxymethyl)-2-bromophenyl)butanoic acid

To a solution of 2-bromo-3-(4-oxobutyl)benzyl acetate (1.2 g, 4 mmol, 1 eq) in tert-butyl alcohol (28.5 mL) was added 2-methyl-2-butene (3 ml) and a solution of NaClO₂ (723.5 mg, 8 mmol, 2 eq) and NaH₂PO₄ (1.872 g, 12 mmol, 3 eq) in water (12 mL) at r.t. The reaction was stirred at rt for 90 min. The reaction was quenched with 1N HCl and extracted with ethyl acetate (EA). The organic phase was washed with brine, dried over anhydrous sodium sulfate, filtered and concentrated in vacuum. The crude was added petroleum ether and stirred for 10 min, then filtered to give 4-(3-(acetoxymethyl)-2-bromophenyl)butanoic acid (1.1 g, yield 87%): Purity by ¹H NMR: 90%.

### Step 10: Preparation of methyl 4-(3-(acetoxymethyl)-2-bromophenyl)butanoate

To a solution of 4-(3-(acetoxymethyl)-2-bromophenyl)butanoic acid (1 g, 3.2 mmol, 1 eq) in DMF (16 mL) was added potassium carbonate (0.877 g, 6.35 mmol, 2 eq). The reaction was stirred at rt for 20 min. To the mixture was added iodomethane (987 uL, 16 mmol, 5 eq), and then stirred at for 1 h. The reaction was quenched with water and extracted with ethyl acetate. The organic phase was washed with 0.5N HCl, saturated sodium carbonate, brine, dried over anhydrous sodium sulfate and filtered. The organic phase was evaporated to give a crude residue. The residue was purified by column chromatography (EA/PE=20%) to give methyl 4-(3-(acetoxymethyl)-2-bromophenyl)butanoate (0.9 mg, yield 89%); Purity by ¹H-NMR: 90%.

### Step 11: Preparation of methyl 4-(3-(acetoxymethyl)-2-(4,4,5,5-tetramethyl-1,3,2-dioxaborolan-2-yl)phenyl)butanoate

To a solution of 4-(3-(acetoxymethyl)-2-bromophenyl)butanoate (300 mg, 0.91 mmol, 1 eq) in dioxane (4.55 mL) was added bis(pinacolato)diboron (277 mg, 1.09 mmol, 1.17 eq), KOAc (384.24 mg, 4.08 mmol, 4.3 eq). The reaction was vacuumed and protected by nitrogen for 15 min. To the reaction mixture was added Pd(dppf)₂C12 (74 mg, 0.09 mmol, 0.1 eq), then vacuumed and backfilled with nitrogen. The reaction was stirred at 85° C. overnight. The reaction was cooled and filtered, then evaporated to give a crude residue that was purified by column chromatography (EA/PE=10%) to give methyl 4-(3-(acetoxymethyl)-2-(4,4,5,5-tetramethyl-1,3,2-dioxaborolan-2-yl)phenyl)butanoate (350 mg, yield 100%); Purity by ¹H-NMR: 70%.

### Step 12: Preparation of 4-(1-hydroxy-1,3-dihydrobenzo[c][1,2]oxaborol-7-yl)butanoic acid

To a solution of methyl 4-(3-(acetoxymethyl)-2-(4,4,5,5-tetramethyl-1,3,2-dioxaborolan-2-yl)phenyl)butanoate (342 mg, 0.9 mmol, 1.0 eq) in methanol was added sodium hydroxide (118 mg, 2.97 mmol, 3.3 eq) at 0° C., then stirred at rt for 2 h. The reaction mixture was concentrated under vacuum at 35° C. The residue was dissolved in HCl/THF (2N, 1.5 mL) and the reaction was stirred at rt for 1 h. The mixture was extracted with ethyl acetate. The organic phase was washed with brine, dried over anhydrous sodium sulfate, and evaporated to give a crude residue that was purified by column chromatography (EA/PE=30%) to give the desired final compound 4-(1-hydroxy-1,3-dihydrobenzo[c][1,2]oxaborol-7-yl)butanoic acid. ¹H-NMR (500 MHz, DMSO-d₆): δ 12.00 (broad s, 1H), 8.89 (broad s, 1H), 7.39-7.36 (m, 1H), 7.20 (d, 1H), 7.10 (d, 1H), 4.96 (s, 2H), 2.80-2.76 (m, 2H), 2.20-2.16 (m, 2H) and 1.84-1.77 (m, 2H) ppm; MS: m/z=219 (M−1, ESI−).

### 14 5-(1-Hydroxy-1,3-dihydrobenzo[c][1,2]oxaborol-7-yl)pentanoic acid

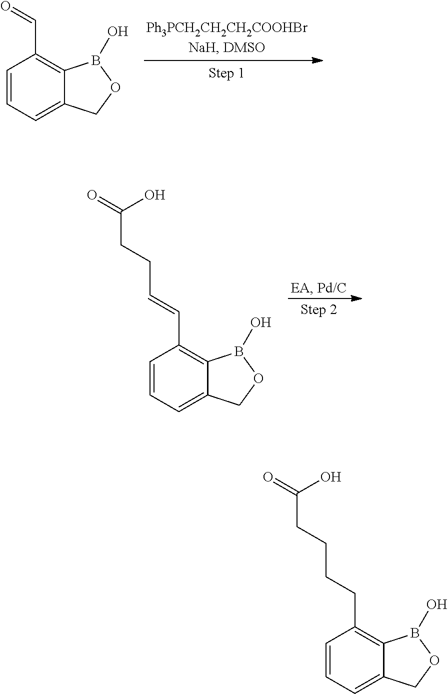

### Step 1: Preparation of (E)-5-(1-hydroxy-1,3-dihydrobenzo[c][1,2]oxaborol-7-yl)pent-4-enoic acid

To a solution of Ph₃PCH₂CH₂CH₂COOH bromide (5.3 g, 12.348 mmol, 4 eq) in DMSO (16 mL) was added a suspension of NaH (1.18 g of 50% oil dispersion) in DMSO (19.7 mL). After being stirred for 20 min at room temperature, a DMSO (6.4 mL) solution of 1-hydroxy-1,3-dihydrobenzo[c][1,2]oxaborole-7-carbaldehyde (500 mg, 3.09 mmol), which was prepared by methods similar to those described in steps 1-5 for 65, was added in one portion. The reaction was stirred at room temperature for 4 h. The reaction was quenched with saturated NH₄Cl, adjusted pH=1-2 with 1N HCl and extracted with ethyl acetate (EA). The organic phase was washed with brine and dried over anhydrous Na₂SO₄. The residue after rotary evaporation was purified by column chromatography to give the desired product (E)-5-(1-hydroxy-1,3-dihydrobenzo[c][1,2]oxaborol-7-yl)pent-4-enoic acid as a solid (120 mg, yield 16%). TLC analysis (silica gel plate, EA:PE=50%): R_{f}=0.3.

### Step 2: Preparation of 5-(1-hydroxy-1,3-dihydrobenzo[c][1,2]oxaborol-7-yl)pentanoic acid

To a solution of (E)-5-(1-hydroxy-1,3-dihydrobenzo[c][1,2]oxaborol-7-yl)pent-4-enoic acid (120 mg, 0.517 mmol) in ethyl acetate (2.6 mL) was added Pd/C (120 mg). The reaction was stirred under hydrogen at rt for 1 h. The reaction was filtered. The residue after rotary evaporation was purified by preparative TLC plate to give the title compound 5-(1-hydroxy-1,3-dihydrobenzo[c][1,2]oxaborol-7-yl)pentanoic acid (30 mg, yield 25%). ¹H NMR (300 MHz, DMSO-d₆): δ 11.90 (s, 1H), 8.85 (s, 1H), 7.37 (t, J=4.5 Hz, 1H), 7.19 (d, J=3 Hz, 1H), 7.09 (d, J=3 Hz, 1H), 4.95 (s, 2H), 2.79 (t, J=4.5 Hz, 2H), 2.19 (t, J=4.5 Hz, 2H), 1.48-1.60 (m, 4H) ppm. Mass: m/z=235 (M+1, ESI+).

### 15 (E)-[3-(1,3-dihydro-1-hydroxy-2,1-benzoxaborol-7-yl)]acrylic acid ethyl ester

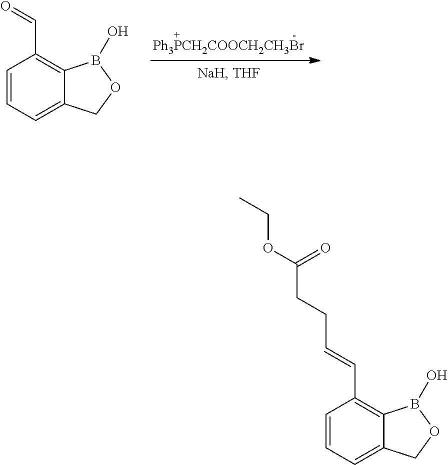

To a mixture of 7-formyl-1-hydroxy-1,3-dihydro-2,1-benzoxaborole (400 mg, 2.47 mmol), (ethyloxycarbonylmethyl) triphenylphosphonium bromide (1060 mg, 2.47 mmol, 1.0 eq) and THF (25 ml) was added under stirring NaH (60% in mineral, 99 mg, 2.47 mmol, 1.0 eq) in portions. The reaction was stirred at room temperature for 12 hours. Another portion of NaH (50 mg, 1.24 mmol, 0.5 eq) was added after cooled to 0° C. After stirring at room temperature for 8 hours, the mixture was quenched with water and acidified to pH=2-3 before it was extracted with ethyl acetate and dried over anhydrous Na₂SO₄. The residue after rotary evaporation was purified by column chromatography and recrystallization to give the title compound (200 mg, 34.9% yield). ¹H NMR (300 MHz, DMSO-d₆): δ 9.33 (s, 1H), 8.10 (d, J=16.2 Hz, 1H), 7.82 (d, J=10 Hz, 1H), 7.52 (t, J=7.5 Hz, 1H), 7.44 (m, 1H), 6.81 (d, J=16.2 Hz, 1H), 5.02 (s, 2H), 4.19 (q, J=7.1 Hz, 2H) and 1.26 (t, J=7.2 Hz, 3H) ppm. Mp 151-152° C.

### 16 7-[(E)-2-carboxyvinyl]-1-hydroxy-1,3-dihydro-2,1-benzoxaborole

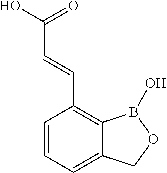

The title compound was prepared by hydrolysis of the corresponding carboxylic ethyl ester, (E)-ethyl 3-(1-hydroxy-1,3-dihydrobenzo[c][1,2]oxaborol-7-yl)acrylate, with a base such as sodium hydroxide followed by neutralization with HCl.

¹H NMR (400 MHz, DMSO-d₆): δ 12.36 (s, 1H), 9.28 (s, 1H), 8.04 (d, J=16.4 Hz, 1H) 7.77 (m, 1H), 7.497 (m, 1H), 7.41 (m, 1H), 6.66 (d, J=16.0 Hz, 1H) and 5.00 (s, 2H) ppm. Mp 219-221° C.

### 17 7-(3′-Hydroxypropyl)-1,3-dihydro-1-hydroxy-2,1-benzoxaborole

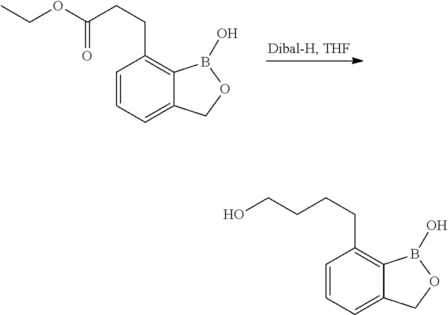

To a solution of [3-(1,3-dihydro-1-hydroxy-2,1-benzoxaborol-7-yl)]propionic acid ethyl ester (500 mg, 2.15 mmol) in THF (20 mL) was added dropwise Dibal-H (1.0M in Hexane, 12.9 mmol, 6.0 eq) at 0° C. The reaction mixture was stirred overnight at room temperature before quenched with 1M HCl at 0° C. The mixture was extracted with ethyl acetate, washed with brine, and dried over anhydrous Na₂SO₄. After rotary evaporation, the residue was purified column chromatography over silica gel to give the title compound (270 mg, 65.5% yield). ¹H NMR (400 MHz, CD₃OD, Sodium was added): δ 7.03 (m, 1H), 6.91 (m, 1H), 6.83 (m, 1H), 4.81 (s, 2H), 3.48 (m, 2H), 2.77 (m, 2H) and 1.84 (m, 2H) ppm. Mp 91-93° C.

### 18 (E)-7-(3-hydroxy-propenyl)-1,3-dihydro-1-hydroxy-2,1-benzoxaborole

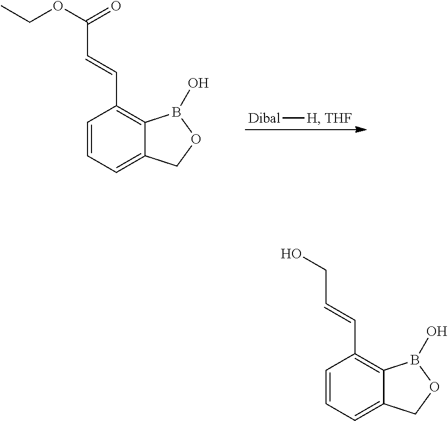

(E)-[3-(1,3-dihydro-1-hydroxy-2,1-benzoxaborol-7-yl)]acrylic acid ethyl ester (0.35 g, 1.51 mmol) was dissolved in anhydrous THF (15 mL) and cooled to −80° C. To this solution under nitrogen was added 1.0 M Dibal-H in hexane (7.6 mL, 7.54 mmol, 5.0 eq). The mixture was allowed to warm to room temperature and stirred overnight. The reaction was quenched with 1M HCl (10 ml), evaporated and extracted with ethyl acetate. The organic layer was washed with water, saturated brine and dried over anhydrous Na₂SO₄. After rotary evaporation, the residue was purified by crystallization to give the title compound (150 mg, 52.4% yield). ¹H NMR (400 MHz, CD₃OD): δ 7.53 (d, J=7.6 Hz, 1H), 7.39 (t, J=7.6 Hz, 1H), 7.21 (d, J=7.2 Hz, 1H), 7.09 (d, J=16.4 Hz, 1H), 6.47 (m, 1H), 5.03 (s, 2H) and 4.23 (dd, J=6.0 &1.6 Hz, 2H) ppm. Mp 211-212° C.

### 19 4-(1-Hydroxy-1,3-dihydrobenzo[c][1,2]oxaborol-7-yl)butan-2-one

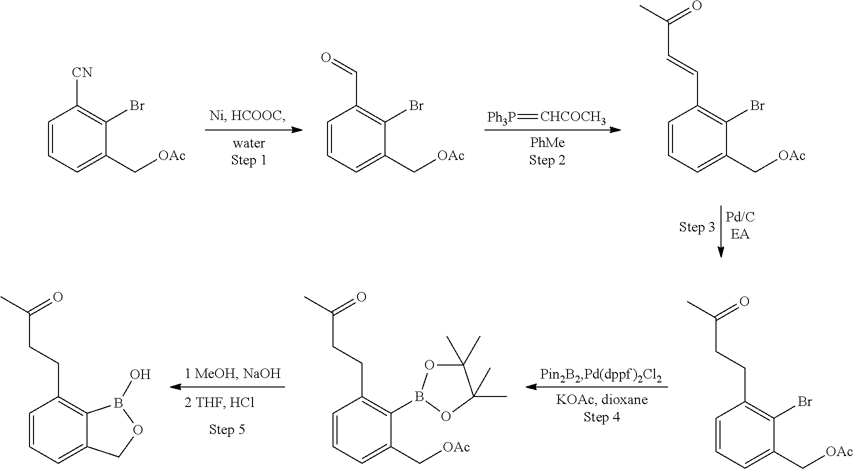

### Step 1: Preparation of 2-bromo-3-formylbenzyl acetate

To Raney Ni (1.6 g, 27.04 mmol, 2.3 eq) was added HCOOH (30 mL), water (9 mL) and 2-bromo-3-cyanobenzyl acetate (3 g, 11.8 mmol, 1 eq). The reaction was stirred at 100° C. for 1 h, cooled and filtered. The residue after rotary evaporation was purified by column chromatography to give the desired aldehyde product (1.5 g, 50% yield).

### Step 2: Preparation of (E)-2-bromo-3-(3-oxobut-1-enyl)benzyl acetate

To a solution of 2-bromo-3-formylbenzyl acetate (1 g, 3.9 mmol, 1 eq) in toluene (30 mL) was added the Wittig reagent Ph₃P═CHC(O)CH₃ (1.48 g, 5 mmol, 1.3 eq). The reaction was stirred at 90° C. for 1 h. The residue after rotary evaporation was purified by column chromatography to give the desired product (0.5 g, 43.5% yield). TLC analysis (silica gel plate, EA: PE=10%): R_{f}=0.2.

### Step 3: Preparation of 2-bromo-3-(3-oxobutyl)benzyl acetate

To a solution of (E)-2-bromo-3-(3-oxobut-1-enyl)benzyl acetate (2.1 g, 7.06 mmol, 1 eq) in ethyl acetate (EA, 35 mL) under nitrogen was added Pd/C (600 mg). The reaction vessel was vacuumed and backfilled with H₂ for 3 times. The reaction was stirred at room temperature for 1 h, filtered and evaporated. The residue was purified by column chromatography to give the desired reduced product (1.1 g, 53% yield). ¹H NMR (300 MHz, DMSO-d₆): δ 7.30 (m, 3H), 5.11 (s, 2H), 2.91 (t, J=4.5 Hz, 2H), 2.76 (t, J=4.5 Hz, 2H), 2.10 (s, 3H), 2.08 (s, 3H) ppm.

### Step 4: Preparation of 3-(3-oxobutyl)-2-(4,4,5,5-tetramethyl-1,3,2-dioxaborolan-2-yl)benzyl acetate

To a solution of 2-bromo-3-(3-oxobutyl)benzyl acetate (500 mg, 1.67 mmol, 1 eq) in dioxane (8.4 mL) was added KOAc (709 mg, 7.22 mmol, 4.3 eq), bis(pinacolato)diboron (640 mg, 2.52 mmol, 1.5 eq) and Pd(dppf)₂Cl₂ (137 mg, 0.167 mmol, 0.1 eq). The reaction vessel was vacuumed and backfilled by N₂ for 3 times. The reaction was stirred at 103° C. overnight. The reaction was filtered and evaporated. The residue was purified by column chromatography to give the desired product (600 mg, 100% yield).

### Step 5: Preparation of 4-(1-hydroxy-1,3-dihydrobenzo[c][1,2]oxaborol-7-yl)butan-2-one

To a solution of 3-(3-oxobutyl)-2-(4,4,5,5-tetramethyl-1,3,2-dioxaborolan-2-yl)benzyl acetate (580 mg, 1.67 mmol, 1 eq) in MeOH (5 mL) was added NaOH (154 mg, 3.86 mmol, 2.3 eq). The reaction was stirred at room temperature for 1 h and then rotary evaporated. THF (2.5 mL) and 2N HCl (2.4 mL) were added. The reaction was stirred at room temperature for half an hour and then extracted with ethyl acetate. The organic phase was washed with brine and dried over anhydrous Na₂SO₄. The residue after rotary evaporation was purified by column chromatography to give the final desired title compound 4-(1-hydroxy-1,3-dihydrobenzo[c][1,2]oxaborol-7-yl)butan-2-one as a slight yellow solid (50 mg, 15% yield). ¹H NMR (300 MHz, DMSO-d₆): δ 8.92 (s, 1H), 7.36 (t, J=4.5 Hz, 1H), 7.20 (d, J=3 Hz, 1H), 7.12 (d, J=3 Hz, 1H), 4.95 (s, 2H), 2.9 (t, J=4.5 Hz, 2H), 2.76 (t, J=4.5 Hz, 2H), 2.08 (s, 3H) ppm; HPLC purity: 99.6% at 220 nm and 100% at 254 nm; MS: m/z=227.2 (M+23, ESI+).

### 20 (E)-[3-(1,3-dihydro-1-hydroxy-2,1-benzoxaborol-7-yl)-1-phenyl]propenone

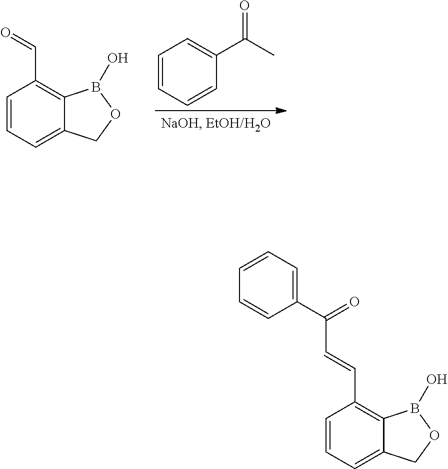

To a mixture of acetophenone (0.22 mL, 222 mg, 1.85 mmol, 1.0 eq), ethanol (5 ml), and water (8 ml) was added NaOH (296 mg, 7.41 mmol, 4.0 eq). After stirring for 5 minutes 7-formyl-1-hydroxy-1,3-dihydro-2,1-benzoxaborole (300 mg, 1.85 mmol, 1.0 eq) was added in portions. The reaction was stirred at room temperature overnight before quenched with 6M HCl to pH=2 under ice-bath. The mixture was evaporated and extracted with ethyl acetate and dried over anhydrous Na₂SO₄. The residue after rotary evaporation was purified by column chromatography and recrystallization to give the title compound (240 mg, 49.1% yield). ¹H NMR (300 MHz, DMSO-d₆): δ 9.39 (s, 1H), 8.14 (m, 5H), 7.68 (t, J=7.4 Hz, 1H), 7.58 (t, J=8 Hz, 3H), 7.48 (m, 1H) and 5.05 (s, 2H) ppm. Mp 136-137° C.

### 21 3-(1-Hydroxy-1,3-dihydrobenzo[c][1,2]oxaborol-7-yl)propanamide

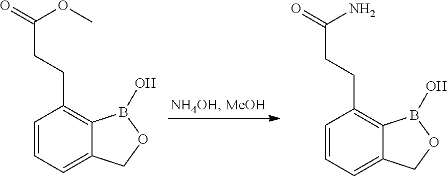

To a solution of methyl 3-(1-hydroxy-1,3-dihydrobenzo[c][1,2]oxaborol-7-yl)propanoate (1 g, 4.5 mmol, 1 eq) in MeOH (22 mL) was added NH₄OH (20 mL, 337.5 mmol, 75 eq). The reaction was stirred at 50° C. overnight. The reaction mixture was evaporated and ethyl acetate (100 mL) was added. The organic phase was washed with 0.1N HCl, then brine, dried over anhydrous Na₂SO₄ and filtered. The residue after rotary evaporation was purified by stirring with Et₂O to give the desired amide compound as white solid (700 mg, 82% yield). ¹H NMR (300 MHz, DMSO-d₆): δ 8.98 (s, 1H), 7.38 (t, J=7.5 Hz, 1H), 7.24 (broad s, 1H), 7.21 (d, J=4.5 Hz, 2H), 7.14 (d, J=4.5 Hz, 1H), 6.76 (broad s, 1H), 4.95 (s, 2H), 2.98 (t, J=9 Hz, 2H), 2.37 (t, J=9 Hz, 2H) ppm. Mass: m/z=206.5 (M+1, ESI+).

### 22 7-[2′-(Ethylcarbamoyl)ethyl]-1,3-dihydro-1-hydroxy-2,1-benzoxaborole

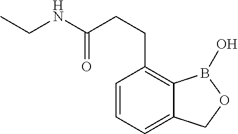

This compound can be prepared by using the same protocol as 7-[2′-(phenylcarbamoyl)ethyl)-1,3-dihydro-1-hydroxy-2,1-benzoxaborole but using ethylamine instead of aniline.

### 23 7-[2′-(Tert-butylcarbamoyl)ethyl]-1,3-dihydro-1-hydroxy-2,1-benzoxaborole

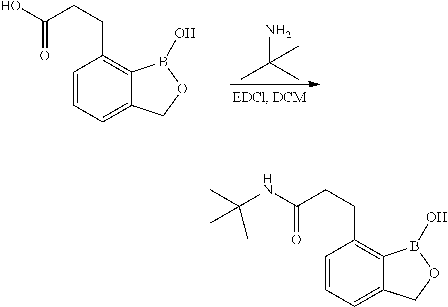

7-(2-Carboxyethyl)-1,3-dihydro-1-hydroxy-2,1-benzoxaborole (258 mg, 1.25 mmol) was added to SOCl₂ (5 ml). The mixture was heated to 80° C. for 1 hour. After evaporation CH₂Cl₂ (10 ml), TEA (0.35 ml, 2.50 mmol, 2.0 eq) and t-butyl amine (0.27 ml, 2.50 mmol, 2.0 eq) were added to the residue sequentially. The mixture was stirred overnight at room temperature before quenched with 1M HCl (10 ml). Then the mixture was extracted with ethyl acetate, washed with brine, and dried over anhydrous Na₂SO₄. The residue after rotary evaporation was purified by column chromatography over silica gel to give the title compound (90 mg, 27.6% yield). ¹H NMR (300 MHz, CDCl₃): δ 8.04 (s, 1H), 7.36 (m, 1H), 7.19 (m, 1H), 7.10 (m, 1H), 5.47 (s, 1H), 5.04 (s, 2H), 3.11 (t, J=7.2 Hz, 2H), 2.45 (t, J=7.2 Hz, 2H) and 1.33 (s, 9H) ppm. Mp 121-122° C.

### 24 7-{2′-[2″-(Dimethylamino)ethylcarbamoyl]ethyl}-1,3-dihydro-1-hydroxy-2,1-benzoxaborole

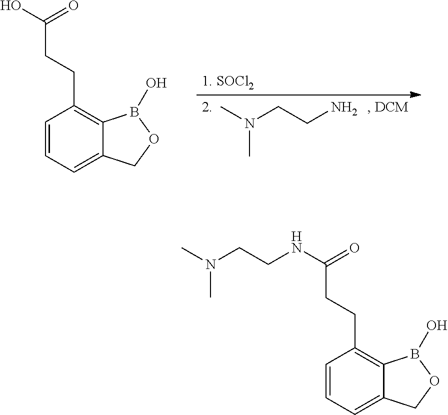

7-(2-Carboxyethyl)-1,3-dihydro-1-hydroxy-2,1-benzoxaborole (380 mg, 1.84 mmol) was added to SOCl₂ (6 ml). The mixture was heated to 80° C. for 1 hour. After evaporation CH₂Cl₂ (15 ml), TEA (0.52 ml, 3.69 mmol, 2.0 eq) and N,N-dimethylethyldiamine (184 ul, 1.84 mmol, 1.0 eq) were added to the residue sequentially. The mixture was stirred overnight at room temperature before quenched with water (5 ml). Then the mixture was extracted with ethyl acetate, washed with brine, and dried over anhydrous Na₂SO₄. The residue after rotary evaporation was purified by column chromatography over silica gel to give the title compound (130 mg, 25.6% yield). ¹H NMR (300 MHz, CD₃OD): δ 7.28 (m, 1H), 7.13 (m, 1H), 7.07 (m, 1H), 5.00 (s, 2H), 3.33 (t, J=6.6 Hz, 2H), 3.04 (t, J=7.5 Hz, 2H), 2.62 (t, J=6.6 Hz, 2H), 2.51 (t, J=7.5 Hz, 2H) and 2.43 (s, 6H) ppm. Mp 132-133° C.

### 25 7-{2′-[3″-(1H-imidazol-1-yl)propylcarbamoyl]ethyl}-1,3-dihydro-1-hydroxy-2,1-benzoxaborole

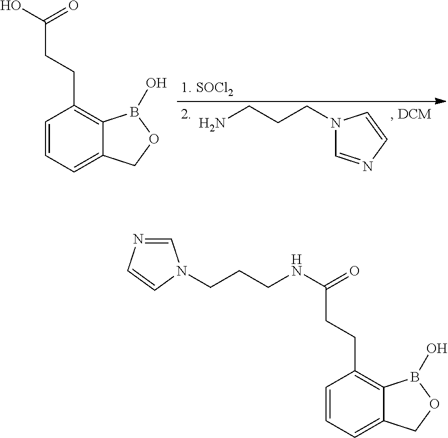

7-(2-Carboxyethyl)-1,3-dihydro-1-hydroxy-2,1-benzoxaborole (258 mg, 1.25 mmol) was added to SOCl₂ (5 ml). The mixture was heated to 80° C. for 1 hour. After evaporation CH₂Cl₂ (10 ml), TEA (0.35 ml, 2.50 mmol, 2.0 eq) and N-(3-Aminopropyl)imidazole (147 ul, 1.25 mmol, 1.0 eq) were added to the residue sequentially. The mixture was stirred overnight at room temperature before quenched by addition of water (10 ml). Then the mixture was extracted with ethyl acetate, washed with brine, and dried over anhydrous Na₂SO₄. The residue after rotary evaporation was purified by column chromatography over silica gel to give the title compound (81 mg, 20.7% yield). ¹H NMR (300 MHz, CD₃OD): δ 7.83 (m, 1H), 7.27 (m, 1H), 7.10 (m, 4H), 5.01 (s, 2H), 3.92 (t, J=6.8 Hz, 2H), 3.13 (t, J=6.5 Hz, 2H), 3.02 (t, J=4.5 Hz, 2H), 2.43 (t, J=7.5 Hz, 2H) and 1.89 (t, J=6.6 Hz, 2H) ppm.

### 26 7-[2′-(Phenylcarbamoyl)ethyl)-1,3-dihydro-1-hydroxy-2,1-benzoxaborole

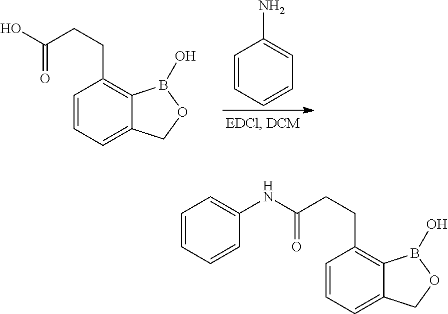

To a mixture of 7-(2-carboxyethyl)-1,3-dihydro-1-hydroxy-2,1-benzoxaborole (258 mg, 1.25 mmol), aniline (125 ul, 1.375 mmol, 1.1 eq) and 5 ml CH₂Cl₂ was added dropwise EDCI (478.8 mg, 2.50 mmol, 2.0 eq) in 5 ml CH₂Cl₂ at 0° C. The mixture was stirred overnight at room temperature then washed with water and brine, and dried over anhydrous Na₂SO₄. The residue after rotary evaporation was purified by crystallization to give the title compound (250 mg, 71.2% yield). ¹H NMR (400 MHz, DMSO-d₆): δ 9.81 (s, 1H), 8.99 (s, 1H), 7.56 (m, 2H), 7.36 (m, 1H), 7.27 (m, 2H), 7.21 (m, 1H), 7.16 (m, 1H), 7.01 (m, 1H), 4.97 (s, 2H), 3.10 (t, J=7.6 Hz, 2H) and 2.64 (t, J=7.6 Hz, 2H) ppm. Mp 181-183° C.

### 27 7-[2′-(4″-Methoxyphenylcarbamoyl)ethyl]-1,3-dihydro-1-hydroxy-2,1-benzoxaborole

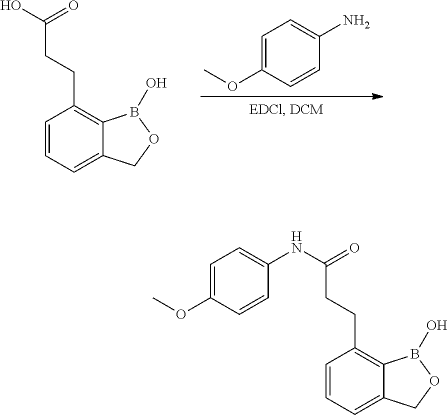

To a mixture of 7-(2-Carboxyethyl)-1,3-dihydro-1-hydroxy-2,1-benzoxaborole (258 mg, 1.25 mmol), p-anisidine (162 mg, 1.315 mmol, 1.05 eq) and 5 ml CH₂Cl₂ was added dropwise EDCI (478.8 mg, 2.50 mmol, 2.0 eq) in 5 ml CH₂Cl₂ at 0° C. The mixture was stirred overnight at room temperature then washed with water and brine, and dried over anhydrous Na₂SO₄. The residue after rotary evaporation was purified by crystallization to give the title compound (115 mg, 29.1% yield). ¹H NMR (300 MHz, DMSO-d₆): δ 9.67 (s, 1H), 8.99 (s, 1H), 7.46 (m, 2H), 7.36 (m, 1H), 7.18 (m, 2H), 6.85 (m, 2H), 4.97 (s, 2H), 3.70 (s, 3H), 3.09 (t, J=7.8 Hz, 2H) and 2.64 (t, J=7.7 Hz, 2H) ppm. Mp 194-196° C.

### 28 {4-[3-(1,3-Dihydro-1-hydroxy-2,1-benzoxaborol-7-yl)propionylamino]benzyl} carbamic acid tert-butyl ester

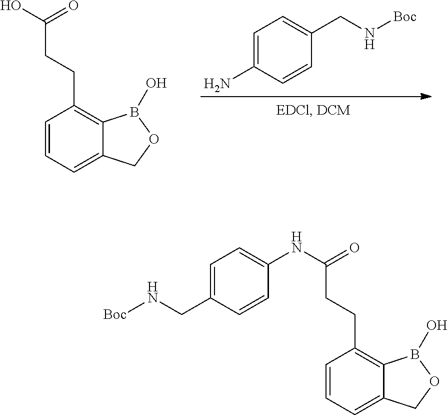

To a mixture of 7-(2-carboxyethyl)-1,3-dihydro-1-hydroxy-2,1-benzoxaborole (258 mg, 1.25 mmol), tert-butyl 4-aminobenzylcarbamate (292 mg, 1.32 mmol, 1.05 eq) and dichloromethane (5 mL) was added EDCI (479 mg, 2.5 mmol, 2.0 eq) in dichloromethane (5 mL) at 0° C. The reaction was allowed to warm to room temperature and stirred overnight. The mixture was washed with water, saturated brine and dried over anhydrous Na₂SO₄. After rotary evaporation, the residue was purified by crystallization to give the title compound (310 mg, 60.4% yield). ¹H NMR (400 MHz, CD₃OD): δ 9.76 (s, 1H), 8.97 (s, 1H), 7.46 (d, J=8.4 Hz, 2H), 7.34 (t, J=7.6 Hz, 1H), 7.32 (q, J=7.5 Hz, 2H), 7.15 (m, 4H), 4.94 (s, 2H), 4.02 (d, J=6 Hz, 2H), 3.06 (t, J=7.8 Hz, 2H), 2.60 (t, J=7.8 Hz, 2H) and 1.36 (s, 9H) ppm. Mp 200-201° C.

### 29 7-{2′-[4″-(Aminomethyl)phenylcarbamoyl]ethyl}-1,3-dihydro-1-hydroxy-2,1-benzoxaborole

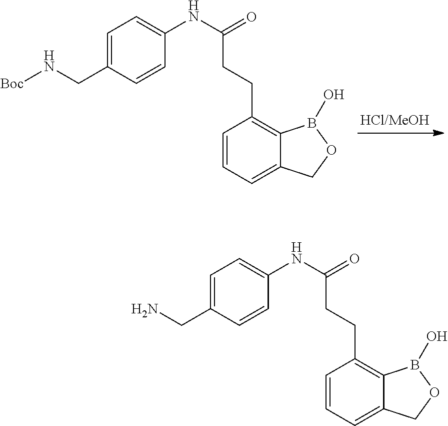

To a solution of {4-[3-(1,3-dihydro-1-hydroxy-2,1-benzoxaborol-7-yl)propionylamino]benzyl} carbamic acid tert-butyl ester (256 mg, 0.62 mmol) in 10 ml methanol at 0° C. was added HCl in methanol (3M, 16 ml). The reaction mixture was stirred for 6 hours at room temperature. After evaporation the residue was dissolved in 1M NaOH and washed with ethyl acetate. The aqueous phase was acidified to pH=7 and extracted and Ethyl acetate. The organic phase was dried over anhydrous Na₂SO₄ and evaporated to give the title compound (100 mg, 51.7% yield). ¹H NMR (300 MHz, DMSO-d₆): δ 9.73 (s, 1H), 9.02 (s, 1H), 7.48 (m, 2H), 7.36 (m, 1H), 7.19 (m, 4H), 4.97 (s, 2H), 3.65 (s, 2H), 3.09 (t, J=7.8 Hz, 2H) and 2.62 (t, J=7.8 Hz, 2H) ppm. Mp 137-138° C.

### 30 3-(1-Hydroxy-1,3-dihydrobenzo[c][1,2]oxaborol-7-O—N-(methylsulfonyl) propanamide

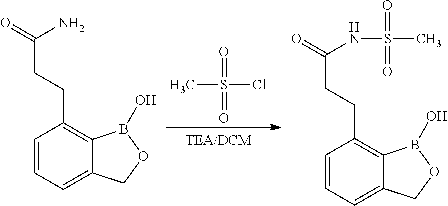

The title compound may be prepared from 3-(1-hydroxy-1,3-dihydrobenzo[c][1,2]oxaborol-7-yl)propanamide and methanesulfonyl chloride in the presence of a base such as triethylamine.

### 31 N-(cyclopropylsulfonyl)-3-(1-hydroxy-1,3-dihydrobenzo[c][1,2]oxaborol-7-yl)propanamide

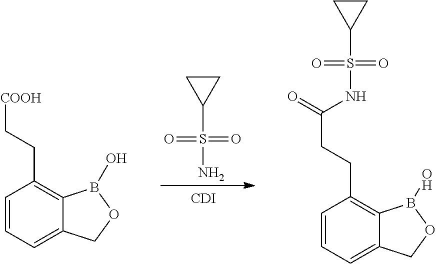

3-(1-Hydroxy-1,3-dihydrobenzo[c][1,2]oxaborol-7-yl)propanoic acid (0.377 g, 1.83 mmol) and CDI (0.892 g, 5.5 mmol) in THF (15 mL) were heated to reflux for 1 h. After being cooled down to r.t, the resulting solution was transferred by syringe into a solution of cyclopropanesulfonamide (0.667 g, 5.5 mmol) in THF (5 mL), followed by addition of DBU (0.56 g, 3.66 mmol). The resulting mixture was stirred overnight before being quenched with 1N HCl and extracted with EtOAc (100 mL). The organic layer was concentrated and the residue was purified by preparative HPLC (column: Luna 300×50.0 mm, 10 g; liquid phase: [A-H₂O; B—CH₃CN+0.1% TFA] B %: 18%-48%, 25 min) and freeze-dried to afford the title compound (270 mg, yield 48.2%). ¹H NMR (400 MHz, DMSO-d₆) δ 11.57 (s, 1H), 8.97 (s, 1H), 7.39-7.34 (m, 1H), 7.22-7.20 (d, 1H), 7.14-7.12 (d, 1H), 4.95 (s, 1H), 3.03-2.99 (t, 2H), 2.92-2.89 (m, 1H), 2.61-2.57 (t, 2H), 1.05-1.03 (m, 4H) ppm. HPLC purity: 99.58% at 220 nm and 100% at 254 nm.

### 32 3-(1-Hydroxy-1,3-dihydrobenzo[c][1,2]oxaborol-7-yl)-N-sulfamoylpropanamide

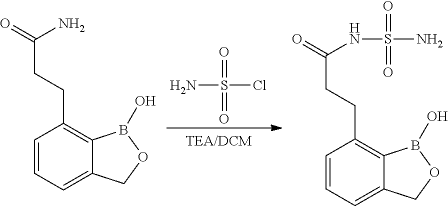

The title compound may be prepared from 3-(1-hydroxy-1,3-dihydrobenzo[c][1,2]oxaborol-7-yl)propanamide and sulfamoyl chloride in the presence of a base such as triethylamine.

### 33 7-[3′-(4″-Methylpiperazin-1″-yl)-3′-oxopropyl]-1,3-dihydro-1-hydroxy-2,1-benzoxaborole

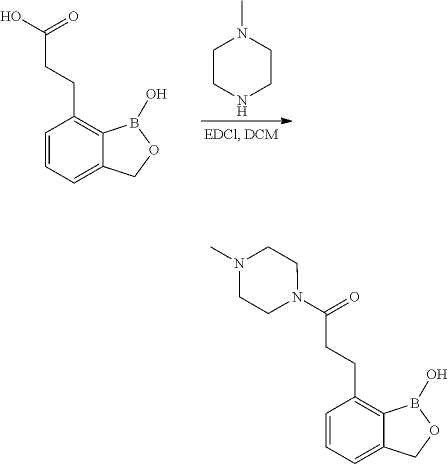

To a mixture of 7-(2-carboxyethyl)-1,3-dihydro-1-hydroxy-2,1-benzoxaborole (258 mg, 1.25 mmol), N-methylpiperazine (153 ul, 1.375 mmol, 1.1 eq) and 5 ml CH₂Cl₂ was added dropwise EDCI (478.8 mg, 2.50 mmol, 2.0 eq) in 5 ml CH₂Cl₂ at 0° C. The mixture was stirred overnight at room temperature then washed with water and brine, and dried over anhydrous Na₂SO₄. The residue after rotary evaporation was purified by crystallization to give the title compound (195 mg, 54.2% yield). ¹H NMR (400 MHz, DMSO-d₆): δ 8.98 (s, 1H), 7.36 (m, 1H), 7.21 (m, 1H), 7.15 (m, 1H), 4.96 (s, 2H), 3.42 (m, 4H), 2.97 (t, J=8.0 Hz, 2H), 2.59 (t, J=8.0 Hz, 2H), 2.21 (m, 4H) and 2.15 (s, 3H) ppm. Mp 156-158° C.

### 34 3-(1-Hydroxy-1,3-dihydrobenzo[c][1,2]oxaborol-7-yl)propanenitrile

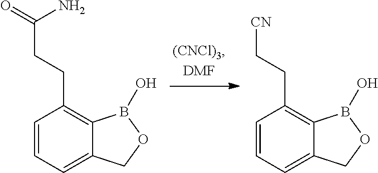

To a solution of 3-(1-hydroxy-1,3-dihydrobenzo[c][1,2]oxaborol-7-yl)propanamide (700 mg, 3.414 mmol, 1 eq) in DMF (17 mL) was added (CNCl)₃ (945 mg, 5.1 mmol, 1.5 eq). The reaction was stirred at room temperature overnight. The reaction was quenched with water and extracted with ethyl acetate (EA). The organic phase was washed with brine, dried over anhydrous Na₂SO₄ and filtered. The residue after rotary evaporation was purified by column chromatography to give the desired cyano product as white solid (370 mg, 58% yield). ¹H NMR (300 MHz, DMSO-d₆): δ 9.07 (s, 1H), 7.41 (t, J=4.5 Hz, 1H), 7.27 (d, J=3 Hz, 1H), 7.21 (d, J=3 Hz, 1H), 4.98 (s, 2H), 3.06 (t, J=4.5, 2H), 2.80 (t, J=4.5, 2H) ppm. Mass: m/z=186.2 (M−1, ESI−).

### 35 7-(2-(1H-Tetrazol-5-yl)ethyl)benzo[c][1,2]oxaborol-1(3H)-ol

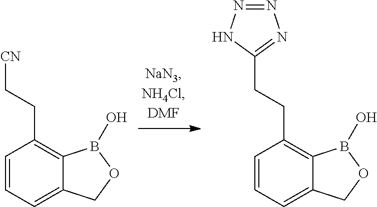

To a solution of 3-(1-hydroxy-1,3-dihydrobenzo[c][1,2]oxaborol-7-yl)propanenitrile (130 mg, 0.695 mmol, 1 eq) in DMF (3.5 mL) was added NaN₃ (76.8 mg, 1.18 mmol, 1.7 eq) and NH₄Cl (63.13 mg, 1.18 mmol, 1.7 eq). The reaction was stirred at 95° C. for 3 d. The reaction was quenched with 0.5N HCl and extracted with ethyl acetate. The organic phase was washed with brine and dried over anhydrous Na₂SO₄. The residue after rotary evaporation was purified by preparative TLC plate to give the title product 7-(2-(1H-tetrazol-5-yl)ethyl)benzo[c][1,2]oxaborol-1(3H)-ol (20 mg, 12.5% yield). ¹H NMR (300 MHz, DMSO-d₆): δ 15.9 (broad s, 1H), 8.96 (s, 1H), 7.36-7.24 (m, 1H), 7.20 (d, J=9 Hz, 1H), 7.04 (d, J=6 Hz, 1H), 4.97 (s, 2H), 3.40 (m, 4H) ppm. Mass: m/z=231 (M+1, ESI+) and 229 (M−1, ESI−).

### 36 5-(2-(1-Hydroxy-1,3-dihydrobenzo[c][1,2]oxaborol-7-yl)ethyl)thiazolidine-2,4-dione

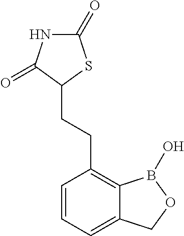

The title compound may be prepared by the following scheme.

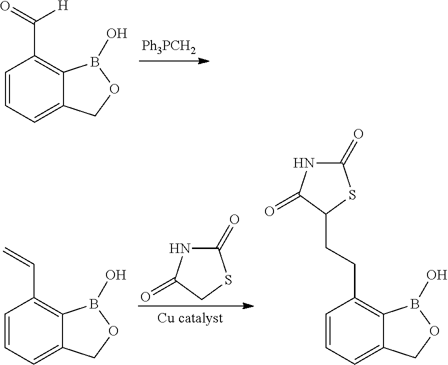

### 37 5-((1-Hydroxy-1,3-dihydrobenzo[c][1,2]oxaborol-7-yl)methyl)thiazolidine-2,4-dione

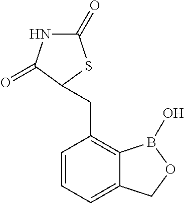

The title compound may be prepared by the following scheme.

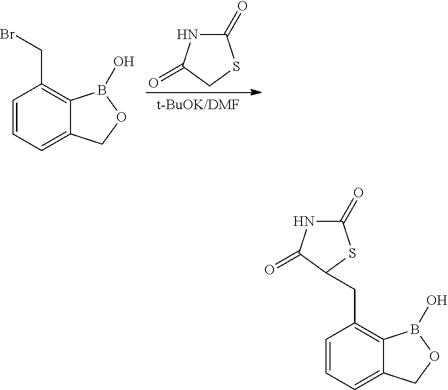

### 38 7-Aminomethyl-1,3-dihydro-1-hydroxy-2,1-benzoxaborole

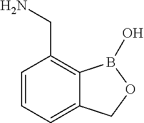

This compound can be prepared by contacting 7-cyano-1,3-dihydro-1-hydroxy-2,1-benzoxaborole with lithium aluminum hydride in THF. ¹H NMR (300 MHz, DMSO-d₆): δ 8.30 (s, 1H), 7.35 (m, 3H), 4.95 (s, 2H), 4.18 (s, 2H). MS: m/z=164 (M+1, ESI+).

### 39 7-(3-Aminopropyl)benzo[c][1,2]oxaborol-1(3H)-ol HCl salt

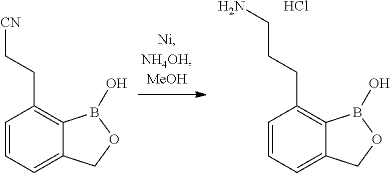

To a solution of 3-(1-hydroxy-1,3-dihydrobenzo[c][1,2]oxaborol-7-yl)propanenitrile (70 mg, 0.37 mmol, 1 eq) in MeOH (2 mL) was added Ni (21 mg) and NH₄OH (140 uL). The reaction flask was vacuumed and backfilled with H₂ for 3 times. The reaction was stirred at room temperature for 1 hour. The reaction was filtered and evaporated. The residue was purified by preparative TLC plate and treated with HCl to give the title compound 7-(3-aminopropyl)benzo[c][1,2]oxaborol-1(3H)-ol HCl salt (19 mg, 27% yield). ¹H NMR (300 MHz, DMSO-d₆): δ 9.00 (s, 1H), 7.86 (broad s, 3H), 7.41 (t, J=4.5 Hz, 1H), 7.24 (d, J=3 Hz, 1H), 7.14 (d, J=1.5 Hz, 1H), 4.98 (s, 2H), 2.84-2.73 (m, 4H), 1.85 (t, J=4.5 Hz, 2H) ppm. Mass: m/z=193 (M−1, ESI−).

### 40 7-[(Propylamino)methyl]-1-hydroxy-1,3-dihydro-2,1-benzoxaborole

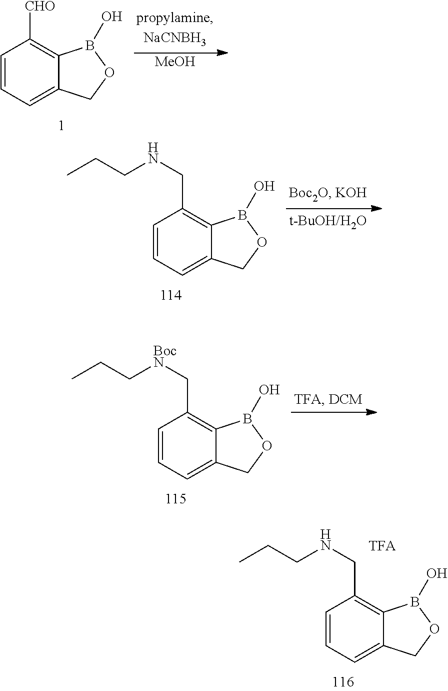

To a mixture of 7-formyl-1-hydroxy-1,3-dihydro-2,1-benzoxaborole (510 mg, 3.145 mmol), MgS₄ (600 mg) and propylamine (743.6 mg, 12.48 mmol, 4.0 eq) in methanol (50 mL) was added NaCNBH₃ (790 mg, 12.58 mmol, 4.0 eq). The mixture was stirred overnight before quenched by the addition of H₂O (5 mL). The mixture was acidified to pH 3-4 with concentrated hydrochloric acid, evaporated under reduced pressure and extracted with ethyl acetate. The organic phase was dried over anhydrous Na₂SO₄ and evaporated to give the crude product 114 (430 mg).

To a solution of the crude product 114 (329 mg, 1.60 mmol) in t-BuOH (3 mL) was added KOH (197.5 mg, 3.52 mmol, 2.2 eq) in water (3.6 mL). To the mixture was added Boc₂O (384.12 mg, 1.76 mmol, 1.1 eq) at 0° C. and the mixture was stirred at room temperature for 2.5 hours before extracted with ethyl acetate. The organic phase was dried over anhydrous Na₂SO₄ and evaporated to give the crude product which was purified by column chromatography to give compound 115 (213 mg).

To a solution of compound 115 (100 mg) in CH₂Cl₂ (2 mL) was added trifluoroacetic acid (2 mL) at 0° C. After the mixture was stirred at room temperature for 1 hour it was evaporated to give the crude product. The crude product was washed by Et₂O and purified by prep-HPLC to give compound 116 (60 mg, 57.0% yield). ¹H NMR (400 MHz, CD₃OD): δ 7.55 (m, 1H), 7.47 (m, 1H), 7.42 (m, 1H), 5.14 (s, 2H), 4.34 (s, 2H), 3.04 (t, J=7.8 Hz, 2H), 1.76 (m, 2H) and 1.03 (t, J=7.4 Hz, 3H) ppm.

### 41 7-[(Aminoethylamino)methyl]-1-hydroxy-1,3-dihydro-2,1-benzoxaborole

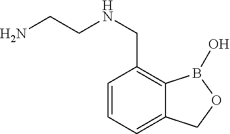

This compound can be prepared by using the same protocol as 7-{[4-(hydroxymethyl)piperidin-1-yl]methyl}-1-hydroxy-1,3-dihydro-2,1-benzoxaborole but using 4-ethoxycarbonyl piperadine instead of (piperidin-4-yl)methanol. ¹H NMR (400 MHz, D₂O): δ 7.37 (t, J=7.2 Hz, 1H), 7.25 (d, J=7.6 Hz, 1H), 7.18 (d, J=7.2 Hz, 1H), 4.99 (s, 2H), 3.91 (s, 2H) and 3.10-2.92 (m, 4H) ppm. (HCl salt).

### 42 7-[(2-Hydroxyethylamino)methyl]-1-hydroxy-1,3-dihydro-2,1-benzoxaborole

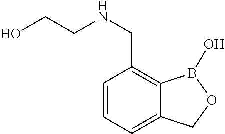

This compound can be prepared by using the same protocol as 7-{[4-(hydroxymethyl)piperidin-1-yl]methyl}-1-hydroxy-1,3-dihydro-2,1-benzoxaborole but using 2-hydroxyethylamino instead of (piperidin-4-yl)methanol. Title compound data: ¹H NMR (400 MHz, CD₃OD): δ 7.06-6.93 (m, 3H), 4.85 (s, 2H), 3.73 (s, 2H), 3.69 (t, J=5.6 Hz, 2H) and 2.71 (t, J=5.6 Hz, 2H) ppm. Mp 186-189° C.

### 43 7-[(N-methoxyethylamino)methyl]-1-hydroxy-1,3-dihydro-2,1-benzoxaborole

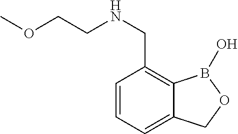

This compound can be prepared by using the same protocol as 7-{[4-(hydroxymethyl)piperidin-1-yl]methyl}-1-hydroxy-1,3-dihydro-2,1-benzoxaborole but using 2-methoxyethylamine instead of (piperidin-4-yl)methanol. ¹H NMR (400 MHz, CDCl₃): δ 7.54 (t, J=7.6 Hz, 1H), 7.47 (d, J=8 Hz, 1H), 7.40 (d, J=7.6 Hz, 1H), 5.15 (s, 2H), 4.36 (s, 2H), 3.65 (t, J=4.8 Hz, 2H), 3.41 (s, 3H) and 3.25 (t, J=4.8 Hz, 2H) ppm. (TFA salt).

### 44 {2-[(1,3-Dihydro-1-hydroxy-2,1-benzoxaborol-7-ylmethylene)-amino]-ethyl}-carbamic acid tert-butyl ester complexed with (2-aminoethyl)-carbamic acid tert-butyl ester

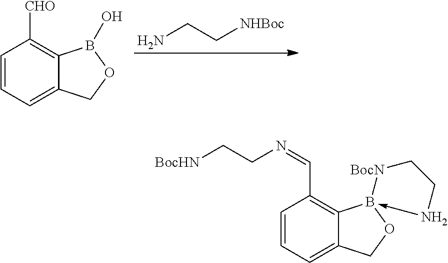

To a mixture of 7-formyl-1-hydroxy-1,3-dihydro-2,1-benzoxaborole (258 mg, 1.59 mmol) in dichloromethane (10 mL) was added tert-butyl 2-aminoethylcarbamate (0.28 mg, 1.75 mmol, 1.1 eq). The mixture was stirred overnight. After evaporation the residue was dissolved in THF (20 mL) and refluxed for 4 hours before MgSO₄ (400 mg) and tert-butyl 2-aminoethylcarbamate (0.15 mL, 0.55 eq) were added. After it was stirred for 8.5 hours, the mixture was filtered, evaporated to give the title compound (610.8 mg, 86.1% yield). ¹H NMR (300 MHz, CDCl₃): δ 8.34 (s, 1H), 7.50 (m, 3H), 4.86 (b, 2H), 3.80 (t, J=4.2 Hz, 2H), 3.51 (q, J=4.5 Hz, 2H), 3.18 (q, J=4.2 Hz, 2H), 2.81 (t, J=4.5 Hz, 2H), 1.45 (s, 9H) and 1.43 (s, 9H) ppm.

### 45 (E)-(1,3-dihydro-1-hydroxy-2,1-benzoxaborol-7-yl)-O-methyl oxime

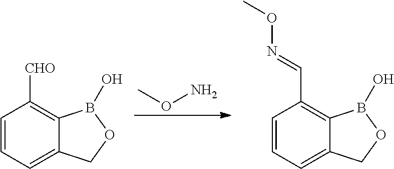

To a mixture of 7-formyl-1-hydroxy-1,3-dihydro-2,1-benzoxaborole (200 mg, 1.24 mmol), O-methylhydroxylamine hydrochloride (125 mg, 1.50 mmol, 1.2 eq) and sodium formate (240 mg, 2.3 mmol, 1.88 eq) was added 88% formic acid (0.95 mL). The mixture was heated to 85° C. and stirred overnight. Then water (10 mL) was added and extracted with ethyl acetate (20 mL×3). The residue after rotary evaporation was purified by column chromatography over silica gel to give the title compound (206.5 mg, 87.9% yield). ¹H NMR (300 MHz, DMSO-d₆): δ 9.13 (s, 1H), 8.52 (s, 1H), 7.69 (d, J=7.6 Hz, 1H), 7.53 (t, J=7.6 Hz, 1H), 7.46 (d, J=7.6 Hz, 1H), 5.04 (s, 2H) and 3.93 (s, 3H) ppm. Mp 65-66° C.

### 46 (E)-(1,3-dihydro-1-hydroxy-2,1-benzoxaborol-7-yl)-O-methyl amine

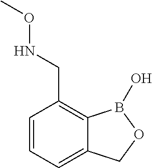

This compound can be prepared by contacting (E)-(1,3-dihydro-1-hydroxy-2,1-benzoxaborol-7-yl)-O-methyl oxime with sodium cyanoborohydride in methanol. ¹H NMR (300 MHz, DMSO-d₆): δ 9.46 (s, 1H), 7.44-7.39 (m, 1H), 7.30-7.27 (d, 1H), 7.15-7.12 (m, 1H), 5.01 (s, 2H), 4.15 (d, 2H) and 3.41 (s, 3H) ppm. MS: m/z=194 (M+1, ESI+).

### 47 (E)-(1,3-dihydro-1-hydroxy-2,1-benzoxaborol-7-yl)-O-benzyl oxime

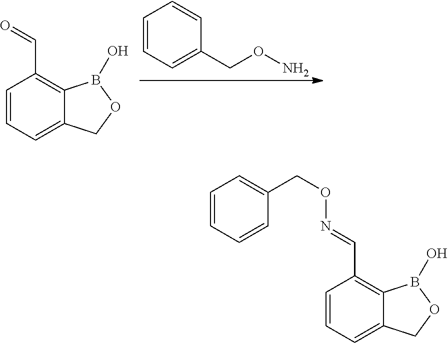

To a mixture of 7-formyl-1-hydroxy-1,3-dihydro-2,1-benzoxaborole (200 mg, 1.24 mmol), O-benzylhydroxylamine hydrochloride (240 mg, 1.50 mmol, 1.2 eq) and sodium formate (0.48 g, 7.1 mmol, 5.7 eq) was added 88% formic acid (1.9 mL). The mixture was heated to 85° C. and stirred overnight. Then water (10 mL) was added and extracted with ethyl acetate (20 mL×3). The residue after rotary evaporation was purified by column chromatography over silica gel to give the title compound (222 mg, 59.3% yield). ¹H NMR (300 MHz, DMSO-d₆): δ 9.06 (s, 1H), 8.58 (s, 1H), 7.67 (d, J=5.4 Hz, 1H), 7.51 (t, J=5.4 Hz, 1H), 7.39 (m, 6H), 5.19 (s, 2H) and 5.02 (s, 2H) ppm. Mp 56-58° C.

### 48 7-[(N-methyl-N-(2-aminoethyl)amino)methyl]-1-hydroxy-1,3-dihydro-2,1-benzoxaborole

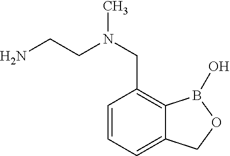

This compound can be prepared by using the same protocol as 7-{[4-(hydroxymethyl)piperidin-1-yl]methyl}-1-hydroxy-1,3-dihydro-2,1-benzoxaborole but using N-methyl-N-(2-aminoethyl)amine instead of (piperidin-4-yl)methanol. ¹H NMR (400 MHz, CD₃OD): δ 7.59-7.44 (m, 3H), 5.13 (s, 2H), 4.47 (s, 2H), 3.49 (m, 4H) and 2.85 (s, 3H) ppm. (TFA salt).

### 49 7-[(N-methyl-N-cyclohexylamino)methyl]-1-hydroxy-1,3-dihydro-2,1-benzoxaborole

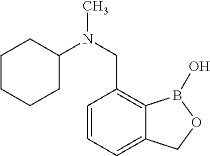

This compound can be prepared by using the same protocol as 7-{[4-(hydroxymethyl)piperidin-1-yl]methyl}-1-hydroxy-1,3-dihydro-2,1-benzoxaborole but using N-methyl-N-cyclohexyl-amine instead of (piperidin-4-yl)methanol. ¹H NMR (400 MHz, CD₃OD): δ 7.21-7.13 (m, 2H), 7.06 (d, J=6.8 Hz, 1H), 4.93 (s, 2H), 4.26 (s, 2H), 3.26-3.17 (m, 1H), 2.54 (s, 3H), 2.12-1.88 (m, 4H) and 1.74-1.60 (m, 6H) ppm.

### 50 7-[(N-propyl-N-acetyl-amino)methyl]-1-hydroxy-1,3-dihydro-2,1-benzoxaborole

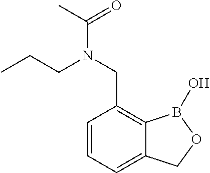

This compound can be prepared by using the same protocol as 7-{[4-(hydroxymethyl)piperidin-1-yl]methyl}-1-hydroxy-1,3-dihydro-2,1-benzoxaborole but using propylamine instead of (piperidin-4-yl)methanol, followed by acetylation by acetic anhydride. ¹H NMR (400 MHz, CDCl₃): δ 8.77 (s, 1H), 7.45-7.15 (m, 3H), 5.03 (s, 2H), 4.75 (s, 2H), 3.19-3.13 (m, 2H), 2.17 (s, 3H), 1.16-1.52 (m, 2H) and 0.913 (t, J=7.1 Hz, 3H) ppm.

### 51 7-[N-acetyl-N-(2-aminoethyl)aminomethyl]-1,3-dihydro-1-hydroxy-2,1-benzoxaborole trifluoroacetate

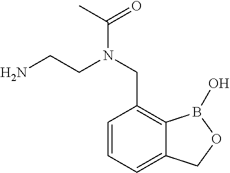

This compound can be prepared by reduction of 42 with sodium cyanoborohydride, followed by acetylation with acetyl anhydride and deprotection with TFA. Title compound data: ¹H NMR (400 MHz, CD₃OD): δ 7.56 (t, J=7.6 Hz, 1H), 7.49 (d, J=4 Hz, 1H), 7.41 (d, J=7.6 Hz, 1H), 5.15 (s, 2H), 4.38 (s, 2H), 3.50 (t, J=5.6 Hz, 2H), 3.19 (t, J=5.6 Hz, 2H) and 1.97 (s, 3H) ppm. Mp 164-166° C.

### 52 7-[(N-acetyl-N—(N-acetylaminoethyl)amino)methyl]-1-hydroxy-1,3-dihydro-2,1-benzoxaborole

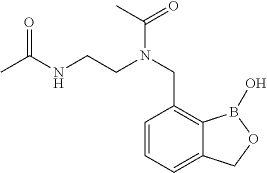

This compound can be prepared by contacting 7-[(aminoethylamino) methyl]-1-hydroxy-1,3-dihydro-2,1-benzoxaborole with at least 2 eq. of acetic anhydride. MS: m/z=291 (M+1, ESI+). ¹H NMR (400 MHz, DMSO-d₆): δ 9.10 (s, 1H), 8.04 (t, J=6.0 Hz, 1H), 7.42 (t, J=7.6 Hz, 1H), 7.29 (t, J=7.6 Hz, 1H), 7.10 (d, J=7.6 Hz, 1H), 4.98 (s, 2H), 4.71 (s, 2H), 3.30-3.10 (m, 4H), 2.09 (s, 3H) and 1.78 (s, 3H) ppm.

### 53 7-[(N-methoxyethyl-N-t-butoxycarbonyl-amino)methyl]-1-hydroxy-1,3-dihydro-2,1-benzoxaborole

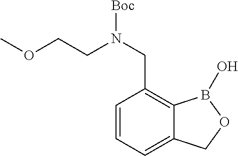

This compound can be prepared by contacting 7-[(N-methoxyethylamino) methyl]-1-hydroxy-1,3-dihydro-2,1-benzoxaborole with t-BOC anhydride. ¹H NMR (400 MHz, CDCl₃): δ 8.68 (s, 1H), 7.42 (t, J=7.2 Hz, 1H), 7.32-7.23 (m, 2H), 5.03 (s, 2H), 4.67 (s, 2H), 3.43 (t, J=5.6 Hz, 2H), 3.33 (s, 3H), 3.26 (t, J=5.6 Hz, 2H) and 1.49 (s, 9H) ppm.

### 54 (S)-7-[2-((hydroxymethyl)pyrrolidin-1-yl)methyl]-1-hydroxy-1,3-dihydro-2,1-benzoxaborole

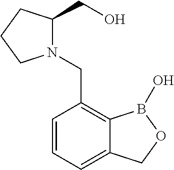

This compound can be prepared by using the same protocol as 7-{[4-(hydroxymethyl)piperidin-1-yl]methyl}-1-hydroxy-1,3-dihydro-2,1-benzoxaborole but using L-prolinol instead of (piperidin-4-yl)methanol. ¹H NMR (400 MHz, CD₃OD, sodium was added): δ 7.05-7.00 (m, 2H), 6.92 (d, J=6.8 Hz, 1H), 4.88-4.78 (m, 2H), 4.22 (d, J=11.6 Hz, 1H), 3.78 (dd, J=11.2 & 3.6 Hz, 1H), 3.43 (dd, J=11.2 & 2.8 Hz, 1H), 3.13 (d, J=12 Hz, 1H), 2.93-2.86 (m, 1H), 2.56-2.46 (m, 1H), 2.27-2.18 (m, 1H) and 1.88-1.55 (m, 4H) ppm.

### 55 (R)-7-[2-((hydroxymethyl)pyrrolidin-1-yl)methyl]-1-hydroxy-1,3-dihydro-2,1-benzoxaborole

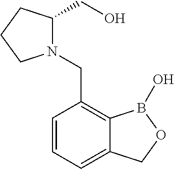

This compound can be prepared by using the same protocol as 7-{[4-(ethoxycarbonyl)piperidin-1-yl]methyl}-1-hydroxy-1,3-dihydro-2,1-benzoxaborole but using D-prolinol instead of (piperidin-4-yl)methanol. Title compound data: ¹H NMR (400 MHz, CD₃OD): δ 7.06-7.00 (m, 2H), 6.91 (d, J=6.8 Hz, 1H), 4.83 (dd, J=19.2 & 13.2 Hz, 2H), 4.21 (d, J=3.6 Hz, 1H), 3.79 (dd, J=11.2 & 3.6 Hz, 1H), 3.43 (dd, J=11.2 & 3.2 Hz, 1H), 3.13 (d, J=12 Hz, 1H) 2.89 (t, J=8 Hz, 1H), 2.52 (m, 1H), 2.22 (m, 1H) and 1.87-1.56 (m, 4H) ppm.

### 56 7-{[4-(Hydroxymethyl)piperidin-1-yl]methyl}-1-hydroxy-1,3-dihydro-2,1-benzoxaborole

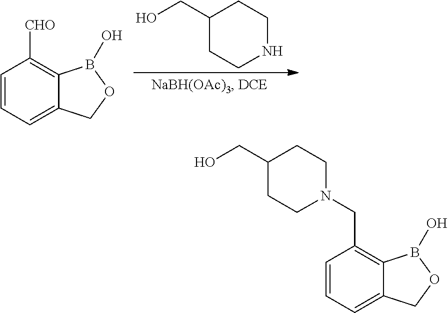

To a mixture of 7-formyl-1-hydroxy-1,3-dihydro-2,1-benzoxaborole (320 mg, 1.98 mmol) and (piperidin-4-yl)methanol (227 mg, 1.98 mmol, 1.0 eq) in 1,2-dichloroethane (10 mL) under ice-bath was added NaBH(OAc)₃ (587.5 mg, 2.77 mmol, 1.4 eq) in portions. The mixture was stirred at room temperature overnight before the reaction was quenched by addition of saturated NaHCO₃ solution and washed by ethyl acetate. The aqueous phase was evaporated to give the crude product which was purified by reversed phase column chromatography to give the title compound (173 mg, 33.7% yield). ¹H NMR (400 MHz, CD₃OD): δ 7.27 (m, 2H), 7.17 (m, 1H), δ 4.83 (s, 2H), 4.29 (s, 2H), 3.48 (m, 4H), 2.93 (m, 2H), 1.96 (m, 1H), 1.82 (m, 2H) and 1.38 (m, 2H) ppm. Mp 109-113° C.

### 57 7-{[4-(Ethoxycarbonyl)piperidin-1-yl]methyl}-1-hydroxy-1,3-dihydro-2,1-benzoxaborole

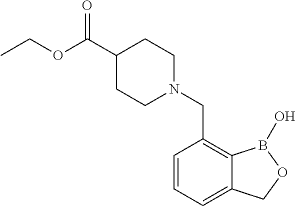

This compound can be prepared by using the same protocol as 7-{[4-(hydroxymethyl)piperidin-1-yl]methyl}-1-hydroxy-1,3-dihydro-2,1-benzoxaborole but using 4-ethoxycarbonyl piperadine instead of (piperidin-4-yl)methanol. ¹H NMR (400 MHz, CD₃OD): δ 7.21-7.13 (m, 2H), 7.06 (d, J=6.8 Hz, 1H), 4.93 (s, 2H), 4.19-4.13 (m, 4H), 3.37-3.05 (m, 2H), 3.01-2.90 (m, 2H), 2.75-2.65 (m, 1H), 2.17-2.07 (m, 2H), 1.97-1.83 (m, 2H) and 1.25 (t, J=7.2 Hz, 3H) ppm.

### 58 7-[(Morpholino)methyl]-1-hydroxy-1,3-dihydro-2,1-benzoxaborole

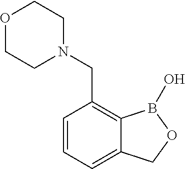

This compound can be prepared by using the same protocol as 7-{[4-(hydroxymethyl)piperidin-1-yl]methyl}-1-hydroxy-1,3-dihydro-2,1-benzoxaborole but using morpholine instead of (piperidin-4-yl)methanol. ¹H NMR (400 MHz, CD₃OD): δ 7.21-7.07 (m, 3H), 4.93 (s, 2H), 4.17 (s, 2H), 3.82 (s, 4H) and 3.09 (s, 4H) ppm.

### 59 7-[(N-methyl-piperizinyl)methyl]-1-hydroxy-1,3-dihydro-2,1-benzoxaborole

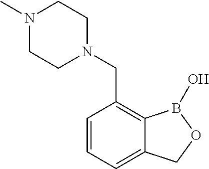

This compound can be prepared by using the same protocol as 7-{[4-(hydroxymethyl)piperidin-1-yl]methyl}-1-hydroxy-1,3-dihydro-2,1-benzoxaborole but using N-methyl piperazine instead of (piperidin-4-yl)methanol. ¹H NMR (400 MHz, CD₃OD): δ 7.21-7.13 (m, 2H), 7.06 (d, J=6.8 Hz, 1H), 4.92 (s, 2H), 4.16 (s, 2H), 3.20-3.02 (m, 4H), 2.82-2.45 (m, 4H) and 2.33 (s, 3H) ppm.

### 60 2-(1-Hydroxy-1,3-dihydrobenzo[c][1,2]oxaborol-7-ylamino)acetic acid

### Step 1: Preparation of 2-bromo-1-methyl-3-nitrobenzene

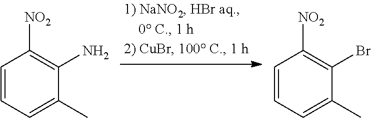

2-Methyl-6-nitrobenzenamine (30.4 g, 0.2 mol) was suspended in water (250 ml) and HBr (100 ml, 40% aq.), and the mixture was heated to reflux for 10 min. Then the mixture was cooled to 0° C. and NaNO₂ (13.8 g, 0.2 mol) in water (80 ml) was added dropwise at such a rate that the temperature did not exceed 5° C. The diazonium solution was stirred for a further 30 min at 0-5° C. and then added slowly to a stirred mixture of CuBr (28.7 g, 0.2 mol) in HBr (80 ml) and water (150 ml) at room temperature. The mixture was stirred at room temperature for 30 min and then on a steam-bath for 1 h. The mixture was washed with saturated NaHCO₃, brine, dried over MgSO₄ and concentrated under vacuum. The residue was purified by column chromatography with petroleum ether as eluent to give a pale yellow solid (25.9 g, yield 60%).

### Step 2: Preparation of 2-bromo-1-(bromomethyl)-3-nitrobenzene

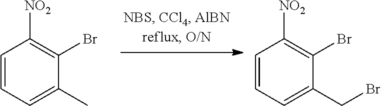

The mixture of 2-bromo-1-methyl-3-nitrobenzene (14.3 g, 0.066 mol), NBS (17.7 g, 0.099 mol) and AIBN (0.3 g, 0.0018 mol) in CCl₄ (250 ml) was refluxed overnight. The mixture was filtered and the filtrate was concentrated to give a red liquid (21 g) as a crude product which was used in the next step without any purification.

### Step 3: Preparation of 2-bromo-3-nitrobenzyl acetate

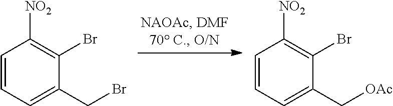

2-bromo-1-(bromomethyl)-3-nitrobenzene (21 g) and NaOAc (16.4 g, 0.2 mol) in DMF (300 ml) was stirred at 70° C. overnight. The mixture was then diluted with water and extracted with ethyl acetate. The combined organic layer was washed with brine, dried over Na₂SO₄ and concentrated under vacuum. The residue was purified by column chromatography with petroleum ether/ethyl acetate (20/1, v/v) as eluent to give a white solid (7.7 g, 42% over two steps).

### Step 4: Preparation of 3-Nitro-2-(4,4,5,5-tetramethyl-1,3,2-dioxaborolan-2-yl)benzyl acetate

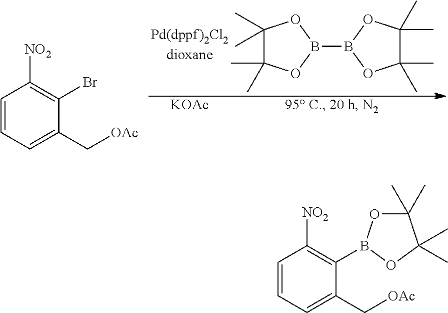

To the solution of 2-bromo-3-nitrobenzyl acetate (16.5 g, 0.06 mol) in 1,4-dioxane (250 ml) was bubbled with nitrogen for 20 min. Potassium acetate (20.6 g, 0.21 mol), 1,1′-bis(diphenylphosphino)ferrocene-palladium(II) dichloride dichloromethane complex (3.92 g, 4.8 mmol) and bis(pinacolato)diboron (22.9 g, 0.09 mol) were added and the reaction mixture was stirred under nitrogen at 95° C. for 20 hours. The reaction mixture was then cooled and was evaporated under vacuum. The residue was partitioned between EtOAc and water. The organic layer was washed with brine, dried over Na₂SO₄ and concentrated under vacuum. The residue was purified by column chromatography with petroleum ether/ethyl acetate (20/1, v/v) as eluent to give a yellow oil (9.9 g, 51%). MS: m/z=322 (M+1, ESI+).

### Step 5: Preparation of 7-nitrobenzo[c][1,2]oxaborol-1(3H)-ol

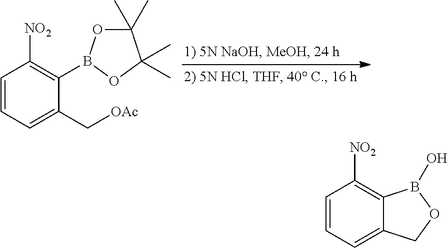

To the solution of 2-(4,4,5,5-tetramethyl-1,3,2-dioxaborolan-2-yl)-3-nitrobenzyl acetate (9.9 g, 0.03 mol) in methanol (300 mL) was added NaOH (5N) (12 mL, 0.06 mol). The reaction mixture was stirred and refluxed under nitrogen for 24 h. The reaction mixture was then concentrated under vacuum and was dissolved in tetrahydrofuran (THF) (100 mL). HCl (5N) (60 mL, 0.3 mol) was added and the reaction mixture was stirred and heated at 40° C. for 16 h. The reaction mixture was cooled, diluted with EtOAc and poured into brine. The separated organic layer was washed with brine, dried over Na₂SO₄ and concentrated under vacuum. The residue was recrystallized from the mixed solvents of ethyl acetate and petroleum ether to give a yellow solid (3.8 g, 71%). ¹H NMR (300 MHz, DMSO-d₆): δ 8.99 (s, 1H), 8.01 (d, 1H), 7.81 (m, 2H), 5.05 (d, 2H) ppm. MS: m/z=180 (M+1, ESI+).

### Step 6: Preparation of 7-aminobenzo[c][1,2]oxaborol-1(3H)-ol

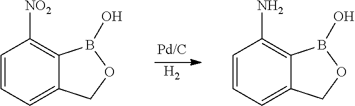

To the solution of 7-nitrobenzo[c][1,2]oxaborol-1(3H)-ol (0.92 g, 5.1 mmol) in methanol (50 ml) was added Pd/C (0.5 g) and the hydrogenation was conducted at one atmosphere and room temperature (r.t.) for 2.5 h to provide the desired product 7-aminobenzo[c][1,2]oxaborol-1(3H)-ol as a solid (0.68 g, yield 88%). ¹H NMR (300 MHz, DMSO-d₆): δ 8.78 (s, 1H), 7.10 (t, 1H), 6.47 (d, 1H), 6.39 (d, 1H), 5.32 (s, 2H), 4.82 (s, 2H) ppm. MS: m/z=150 (M+1, ESI+).

### Step 7: Preparation of ethyl 2-(1-hydroxy-1,3-dihydrobenzo[c][1,2]oxaborol-7-ylamino) acetate

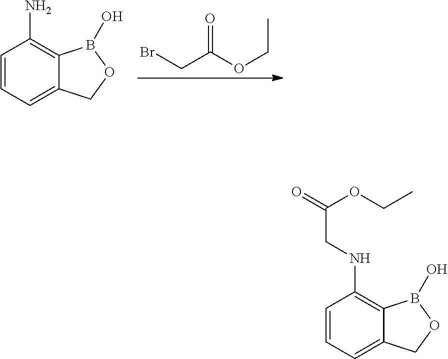

To a mixture of 7-aminobenzo[c][1,2]oxaborol-1(3H)-ol (800 mg, 0.00537 mol) and potassium carbonate (2.23 g, 0.0161 mol) in N,N-dimethyl acetamide (17.9 mL) was added ethyl bromoacetate (0.623 mg, 0.00376 mol). The reaction was stirred overnight at r.t. The mixture was diluted with water and extracted with ethyl acetate. The combined organic layer was washed with 2N HCl, brine, dried over anhydrous Na₂SO₄ and concentrated. The residue was purified by column chromatography (10% EA/DCM) to give the desired product ethyl 2-(1-hydroxy-1,3-dihydrobenzo[c][1,2]oxaborol-7-ylamino) acetate as a solid (380 mg, 30%). ¹H NMR (300 MHz, DMSO-d₆): δ 8.98 (s, 1H), 7.20 (t, J=7.71 Hz, 1H), 6.60 (d, J=3.7 Hz, 1H), 6.25 (d, J=4 Hz, 1H), 5.63 (s, 1H), 4.86 (s, 2H), 4.14 (q. J=10.6 Hz, 2H), 3.8 (s, 2H), 1.20 (t, J=7.09 Hz, 3H) ppm.

### Step 8: Preparation of 2-(1-hydroxy-1,3-dihydrobenzo[c][1,2]oxaborol-7-ylamino)acetic acid

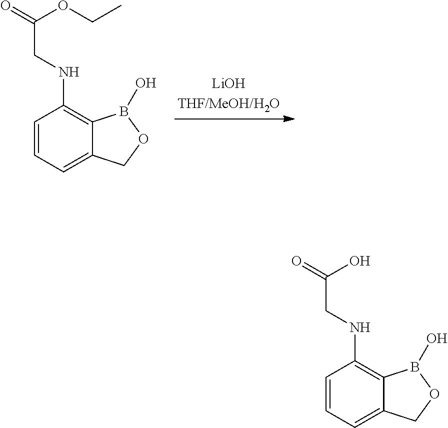

The mixture of ethyl 2-(1-hydroxy-1,3-dihydrobenzo[c][1,2]oxaborol-7-ylamino) acetate (30 mg, 0.127 mmol) and LiOH.H₂O (10.7 mg, 0.255 mmol) in THF:MeOH:H₂O=3:2:1 (0.51 mL total) was stirred for 2 hrs. The mixture was purified by prepared TLC plate to give the desired title compound as a solid (11 mg, yield 42.3%). ¹H NMR (300 MHz, DMSO-d₆): δ 8.99 (s, 1H), 7.19 (s, 1H), 6.53 (d, J=0.66 Hz, 1H), 6.23 (s, 1H), 5.72 (s, 1H), 4.83 (s, 2H), 3.75 (s, 2H) ppm. MS: m/z=205.8 (M−1, ESI−).

### 61 2-((1-Hydroxy-1,3-dihydrobenzo[c][1,2]oxaborol-7-yl)methyl)amino)acetic acid

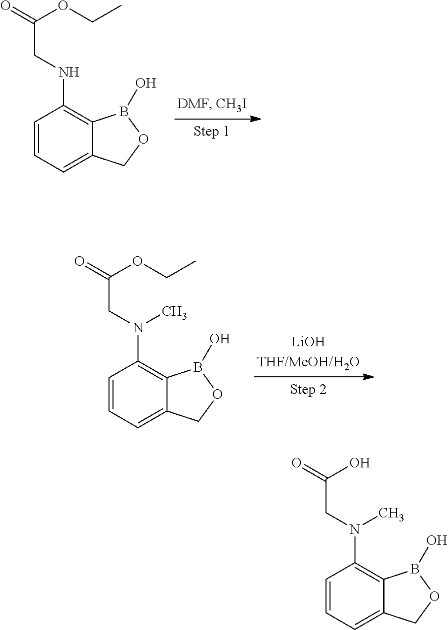

### Step 1: Preparation of ethyl 2-((1-hydroxy-1,3-dihydrobenzo[c][1,2]oxaborol-7-yl)(methyl)amino)acetate

To ethyl 2-(1-hydroxy-1,3-dihydrobenzo[c][1,2]oxaborol-7-ylamino)acetate, prepared in steps 1-7 in 2-(1-hydroxy-1,3-dihydrobenzo[c][1,2]oxaborol-7-ylamino)acetic acid, (100 mg, 0.435 mmol) and K₂CO₃ (176 mg, 1.27 mmol) in DMF (2.1 mL) was added CH₃I (302 mg, 2.12 mmol). The reaction was stirred for 2 h. The mixture was diluted with water and extracted with ethyl acetate. The combined organic layer was washed with 0.5N HCl, brine, dried over Na₂SO₄ and concentrated. The mixture was purified by preparative TLC plate to give the desired product ethyl 2-((1-hydroxy-1,3-dihydrobenzo[c][1,2]oxaborol-7-yl)(methyl)amino)acetate (39 mg, yield 37%) as a solid. ¹H NMR (500 Hz, DMSO-d₆): δ 9.03 (s, 1H), 7.28 (t, J=7.77 Hz, 1H), 6.70 (d, J=3.7 Hz, 1H), 6.57 (d, J=4.1 Hz, 1H), 4.87 (s, 2H), 4.46 (s, 2H), 4.05 (q, J=10.7 Hz, 2H), 2.95 (s, 3H), 1.15 (t, J=4.3 Hz, 3H) ppm. Mass: m/z=250 (M−1, ESI−).

### Step 2: Preparation of 2-((1-hydroxy-1,3-dihydrobenzo[c][1,2]oxaborol-7-yl)(methyl)amino)acetic acid

A mixture of ethyl 2-((1-hydroxy-1,3-dihydrobenzo[c][1,2]oxaborol-7-yl)(methyl)amino)acetate (60 mg, 0.24 mmol) and LiOH.H₂O (20.2 mg, 0.255 mmol) in THF:MeOH:H₂O=3:2:1 (0.936 mL) was stirred for 2 h. The mixture was purified by preparative TLC plate to give 2-((1-hydroxy-1,3-dihydrobenzo[c][1,2]oxaborol-7-yl)(methyl)amino)acetic acid (17 mg, yield 34.9%) as a solid. ¹H NMR (300 Hz, DMSO-d₆): δ 7.26 (t, J=7.7 Hz, 1H), 6.67 (d, J=3.6 Hz, 1H), 6.56 (d, J=4.0 Hz, 1H), 4.86 (s, 2H), 4.39 (s, 2H), 2.96 (s, 3H) ppm. Mass: m/z=220 (M−1, ESI−).

### 62 2-((1-Hydroxy-1,3-dihydrobenzo[c][1,2]oxaborol-7-yl)(acetamido)amino) acetic acid

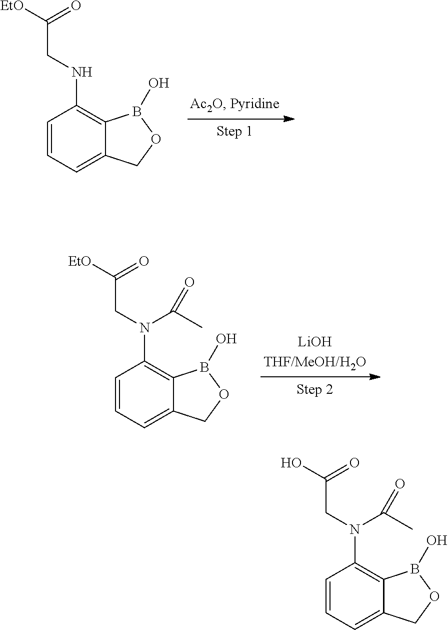

Ethyl 2-(1-hydroxy-1,3-dihydrobenzo[c][1,2]oxaborol-7-yl-amino) acetate can be treated with 2.5 equivalents of acetic anhydride in pyridine for 24 hours at room temperature. The reaction can be quenched with water and extracted with ethyl acetate. The organic phase can be washed with aqueous sodium bicarbonate followed by brine solution and can be dried over anhydrous Na₂SO₄. The residue after rotary evaporation can be purified by preparative TLC plate to give a 50% yield of ethyl 2-((1-hydroxy-1,3-dihydrobenzo[c][1,2]oxaborol-7-yl)(acetamido)amino) acetate. This material can be hydrolyzed to the corresponding carboxylic as follows. A mixture of the ethyl ester (0.24 mmol) and LiOH.H₂O (20.2 mg, 0.255 mmol) in THF:MeOH:H₂O=3:2:1 (0.936 mL) can be stirred for 12 h at room temperature. After aqueous work-up, the organic extract can be purified by preparative TLC plate to give 2-((1-hydroxy-1,3-dihydrobenzo[c][1,2]oxaborol-7-yl)(acetamido)amino)acetic acid.

### 63 1-Hydroxy-1,3-dihydrobenzo[c][1,2]oxaborole-6-carboxylic acid

### 3-Bromo-4-(hydroxymethyl)benzonitrile

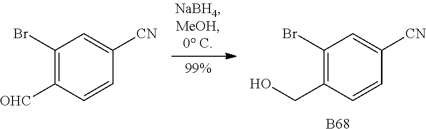

A solution of 3-bromo-4-formylbenzonitrile (1.0 g, 4.8 mmol) in CH₃OH (30 mL) was cooled to 0° C. NaBH₄ (180 mg, 4.8 mmol) was added portionwise. The mixture was allowed to warm to room temperature and stirred at room temperature for 1 h. The mixture was quenched with 1N HCl and concentrated under vacuum. The residue was extracted with ethyl acetate (25 mL*3). The combined organic layers were washed with brine (20 mL), dried (Na₂SO₄) and concentrated under vacuum to give a white solid of the desired compound (1.0 g, 99%). NMR (300 MHz, CDCl₃): δ 7.82 (s, 1H), 7.49-7.71 (m, 2H), 4.75 (s, 2H). LC-MS: 212 (M+1)⁺.

### 1-Hydroxy-1,3-dihydrobenzo[c][1,2]oxaborole-6-carbonitrile

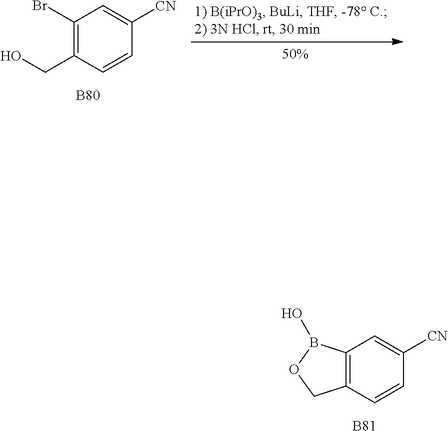

A solution of Intermediate B80 (211 mg, 1.0 mmol) and triisopropyl borate (282 mg, 1.5 mmol) in anhydrous THF (10 mL) at N₂ atmosphere was cooled to −78° C. n-BuLi (0.9 mL, 2.25 mmol) was added dropwise at −78° C. Then the mixture was allowed to warm to room temperature and stirred at room temperature for 1 h. The mixture was quenched with 1N HCl and extracted with ethyl acetate (25 mL*3). The combined organic layers were washed with brine (20 mL), dried (Na₂SO₄) and concentrated under vacuum. The residue was purified by column chromatography (eluting with CH₃OH and EtOAc=1:1) on silica gel to give the desired compound as a yellow solid (80 mg, 50%). ¹H NMR (300 MHz, CDCl₃): δ 9.50 (s, 1H), 8.08 (s, 1H), 7.83-7.92 (m, 1H), 7.61-7.66 (m, 1H), 5.06 (s, 2H). LC-MS: 160 (M+1)⁺.

### 1-Hydroxy-1,3-dihydrobenzo[c][1,2]oxaborole-6-carboxylic acid

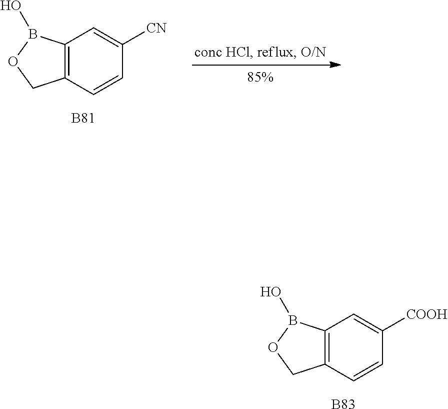

A solution of Intermediate B81 (100 mg, 0.63 mmol) in conc. HCl (10 mL) was refluxed for 3 h and cooled to RT. The mixture was filtrated. The solid was washed with water, dried to give the desired product as a white solid (95 mg, 85%). ¹H NMR (300 MHz, DMSO-d₆): δ 12.92 (s, 1H), 9.36 (s, 1H), 8.10 (s, 1H), 8.05 (d, 1H), 7.54 (d, 1H), 5.08 (s, 2H). LC-MS: 177 (M−1)⁺. Purity on HPLC: 50.5% (214 nm).

### 64 2-(1-Hydroxy-1,3-dihydrobenzo[c][1,2]oxaborol-6-yl) acetic acid

### 1-Hydroxy-1,3-dihydrobenzo[c][1,2]oxaborole-6-carbaldehyde

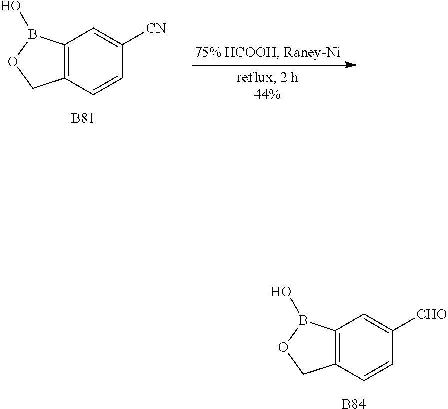

To a solution of B81 (2.0 g, 12.6 mmol) in HCOOH (30 mL) and water (6 mL) was added Raney-Ni (0.82 g, 13.9 mmol) and heated to reflux for 2 h. Then the reaction mixture was cooled to RT and filtered. The filtrate was added water and extracted with EtOAc (30 mL*3). The combined organic layers were washed with brine (50 mL), dried (Na₂SO₄) and concentrated under vacuum. The residue was purified by column chromatography (eluting with CH₃OH and CH₂Cl₂=1:30) on silica gel to give the desired compound as a white solid (0.89 g, 44%). ¹H NMR (300 MHz, DMSO-d₆): δ 10.06 (s, 1H), 9.46 (s, 1H), 8.28 (s, 1H), 8.01 (d, 1H, J=7.8 Hz), 7.64 (d, 1H, J=7.8 Hz), 5.09 (s, 2H). LC-MS: 163 (M+1)⁺.

### Preparation of 2-(1-hydroxy-1,3-dihydrobenzo[c][1,2]oxaborol-6-yl)acetic acid

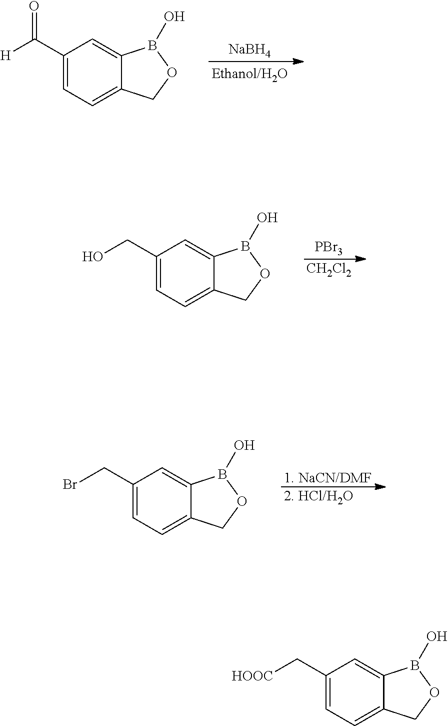

The title compound may be prepared by using the scheme above.

### 65 3-(1-Hydroxy-1,3-dihydrobenzo[c][1,2]oxaborol-6-yl)propanoic acid

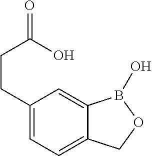

### Step 1: Preparation of 3-bromo-4-methylbenzonitrile

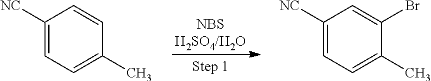

To a mixture of 4-methylbenzonitrile (168 g, 1.46 mol) in H₂SO₄/H₂O (800 mL, v:v=1:1) was added NBS (256 g, 1.43 mol) at 10° C. and stirred for 48 h in the dark. The mixture was filtered and the filter cake was dissolved in ethyl acetate (1000 mL). The organic layer was washed with water and brine, neutralized to pH=7 with NaOH and washed with brine. The organic layer was dried over anhydrous Na₂SO₄ and concentrated in vacuum to give 3-bromo-4-methylbenzonitrile as a yellow solid (276.5 g, 89% yield).

### Step 2: Preparation of 3-bromo-4-(bromomethyl)benzonitrile

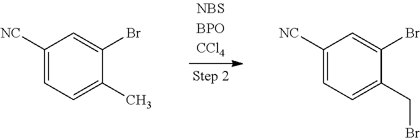

To a solution of 3-bromo-4-methylbenzonitrile (276 g, 1.41 mol) in CCl₄ (3.0 L) was added benzoyl peroxide (BPO, 3.0 g) and NBS (298.0 g, 1.65 mol). The mixture was stirred at refluxing temperature overnight. The mixture was cooled and diluted with DCM, washed with water and concentrated in vacuum to give 3-bromo-4-(bromomethyl)benzonitrile (415 g) as a crude product which was used for next step directly without further purification.

### Step 3: Preparation of 2-bromo-4-cyanobenzyl acetate

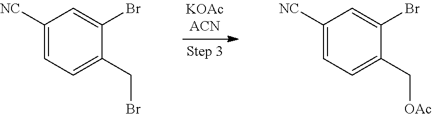

To a solution of the crude 3-bromo-4-(bromomethyl)benzonitrile in CH₃CN (2.0 L) was added KOAc (296 g, 3.02 mol) at 10° C. and the resulting mixture was stirred at 70° C. for 24 h. The solvent was removed in vacuum and the residue was dissolved in ethyl acetate and washed with water. The solution was dried over anhydrous Na₂SO₄, concentrated in vacuum and purified by chromatography on silica gel (PE:EA=20:1) to give 2-bromo-4-cyanobenzyl acetate as a white solid (115 g, yield 32% over two steps).

### Step 4: Preparation of 4-cyano-2-(4,4,5,5-tetramethyl-1,3,2-dioxaborolan-2-yl)benzyl acetate

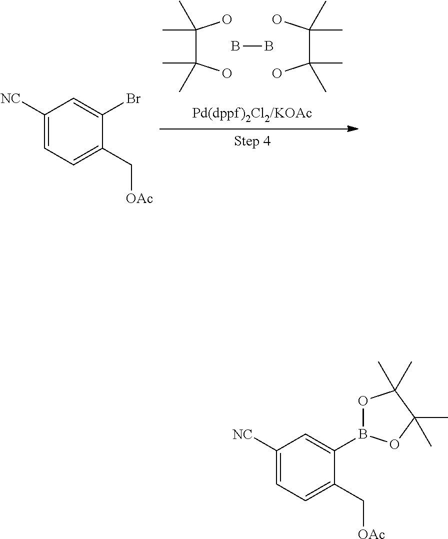

Under a nitrogen atmosphere, 2-bromo-4-cyanobenzyl acetate (150 g, 0.591 mol), KOAc (115.7 g, 1.18 mol), bis(pinacolato)diborane (195 g, 0.768 mol) and Pd(dppf)₂Cl₂ (15 g) were dissolved in dioxane (2 L, degassed before use), and then refluxed for 10 hrs. After starting material was consumed, the mixture was filtered and the filter cake was washed with ethyl acetate. The combined organic solvent was washed with water and brine. The solution was dried over anhydrous Na₂SO₄ and concentrated. The residue was purified by chromatography over silica gel (PE:EA=5:1-2:1) to give a solid. The solid was washed with PE:EA=100:1 to give 4-cyano-2-(4,4,5,5-tetramethyl-1,3,2-dioxaborolan-2-yl)benzyl acetate as a white solid (135.5 g, yield 76%).

### Step 5: Preparation of 1-hydroxy-1,3-dihydrobenzo[c][1,2]oxaborole-6-carbonitrile

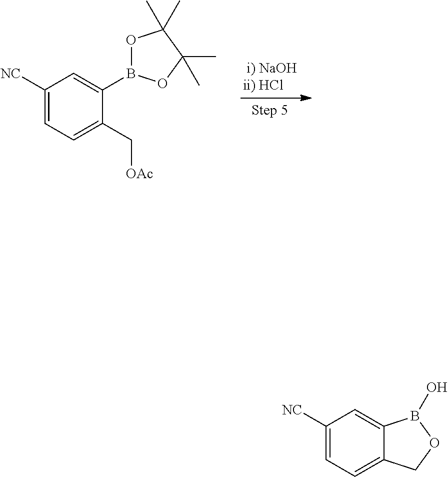

To a solution of 4-cyano-2-(4,4,5,5-tetramethyl-1,3,2-dioxaborolan-2-yl)benzyl acetate (135.5 g, 0.45 mol) in methanol (480 mL) was added a solution of NaOH (40 g, 1.0 mol) in methanol (800 mL) at 30° C. over a period of 1 h. The mixture was stirred for additional 2 h. The solvent was removed in vacuum and the residue was dissolved in a mixed solution of THF (720 mL) and aqueous HCl (2.0 L, 2N). The mixture was stirred at 30° C. for 1 h. After cooling to 15° C., the mixture was filtered and the filter cake was washed with water and petroleum ether to give 1-hydroxy-1,3-dihydrobenzo[c][1,2]oxaborole-6-carbonitrile as a white solid (56.7 g, yield 80%). ¹H NMR (400 MHz, DMSO-d₆): δ 9.53 (s, 1H), 8.10 (s, 1H), 7.93 (m, 1H), 7.66 (m, 1H) and 5.09 (s, 2H) ppm.

### Step 6: Preparation of 1-hydroxy-1,3-dihydrobenzo[c][1,2]oxaborole-6-carbaldehyde

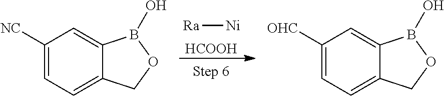

To a solution of 1-hydroxy-1,3-dihydrobenzo[c][1,2]oxaborole-6-carbonitrile (2.0 g, 12.6 mmol) in HCOOH (50 mL) and water (10 mL) was added Raney-Ni (1.0 g). The mixture was stirred under reflux for 5 h and filtered. The filtrate was concentrated to dryness and water was added to give a white solid that was collected by filtration to give 1-hydroxy-1,3-dihydrobenzo[c][1,2]oxaborole-6-carbaldehyde as a white solid (1.5 g, yield 70%). ¹H NMR (400 MHz, DMSO-d₆): δ 10.06 (s, 1H), 9.46 (s, 1H), 8.28 (s, 1H), 8.01 (d, 1H), 7.64 (d, 1H) and 5.09 (s, 2H) ppm.

### Step 7: Preparation of 3-(1-hydroxy-1,3-dihydrobenzo[c][1,2]oxaborol-6-yl)propanoic acid

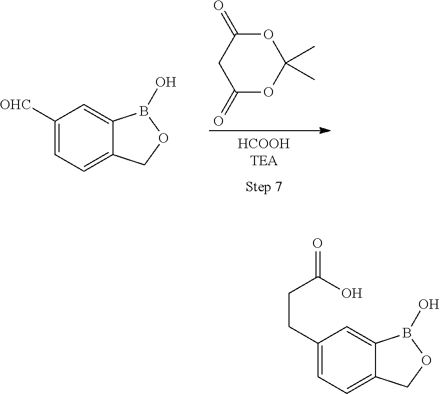

A mixture of 1-hydroxy-1,3-dihydrobenzo[c][1,2]oxaborole-6-carbaldehyde (200 mg, 1.24 mmol, 1.0 eq) and 2,2-dimethyl-1,3-dioxane-4,6-dione (196.5 mg, 1.36 mmol, 1.1 eq) in (HCOOH/TEA=5:2/volume) (1.2 mL) was heated at 110° C. for 2 h. TLC showed no starting material remained. The mixture was poured into ice-water (10 mL) and adjusted to pH=10 with 1N NaOH. The solution was extracted with ethyl acetate twice. The aqueous phase was adjusted pH=2 with 1N HCl, extracted with ethyl acetate for three times. The organic layers were combined, washed with brine, dried over anhydrous Na₂SO₄, filtered and concentrated. The crude product was purified by preparative TLC plate to give the desired final compound 3-(1-hydroxy-1,3-dihydrobenzo[c][1,2]oxaborol-6-yl)propanoic acid as white solid (51 mg, yield 20.4%). ¹H NMR (300 MHz, DMSO-d₆): δ 12.08 (s, 1H), 9.09 (s, 1H), 7.56 (s, 1H), 7.32 (s, 2H), 4.94 (s, 2H), 2.86 (t, J=7.5 Hz, 2H), 2.53 (t, J=8.9 Hz, 2H). MS: m/z=205 (M−1, ESI−). Purity: 95.64% at 220 nm and 95.78% at 254 nm.

### 66 4-(1-Hydroxy-1,3-dihydrobenzo[c][1,2]oxaborol-6-yl)butanoic acid

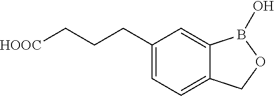

### Step 1: Preparation of (E)-6-(2-methoxyvinyl)benzo[c][1,2]oxaborol-1(3H)-ol

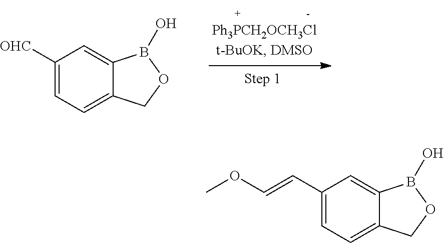

To a solution of Ph₃PCH₂OCH₃ Cl (3.8 g, 11.11 mmol, 3.6 eq) in DMSO (25 mL) was added t-BuOK (1.18 g, 10.50 mmol, 3.4 eq) at 0° C. for 5 min. The solution was stirred at 0° C. under nitrogen for 1 hr. A solution of 1-hydroxy-1,3-dihydrobenzo[c][1,2]oxaborole-6-carbaldehyde (500 mg, 3.08 mmol, 1.0 eq) in DMSO (6 mL) was added over 3 min. The mixture was stirred at room temperature overnight. The reaction mixture was poured into water (60 mL), extracted with ethyl acetate (EA) (2×30 mL), washed with brine and dried over anhydrous sodium sulfate. The solvent was removed to give oil (2.7 g). The oil residue was purified by column chromatography to give a white solid (0.6 g). This material was further purified by preparative TLC plate to give the desired product (E)-6-(2-methoxyvinyl)benzo[c][1,2]oxaborol-1(3H)-ol (195 mg, yield 32%) as a white solid. Mass: m/z=191 (M+1, ESI+), 213 (M+Na), 403 (2M+Na).

### Step 2: Preparation of 2-(1-hydroxy-1,3-dihydrobenzo[c][1,2]oxaborol-6-yl)acetaldehyde

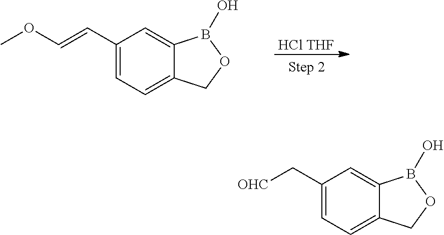

To a solution of (E)-6-(2-methoxyvinyl)benzo[c][1,2]oxaborol-1(3H)-ol (150 mg, 0.789 mmol) in THF (1.3 mL) was added 6N HCl. The reaction mixture was refluxed for 2 h. The mixture was cooled, and then 5 ml water was added. The mixture was extracted with EA (3×20 ml), washed with brine and dried over anhydrous sodium sulfate. The solvent was removed to give the crude product 2-(1-hydroxy-1,3-dihydrobenzo[c][1,2]oxaborol-6-yl)acetaldehyde (200 mg) as oil.

### Step 3: Preparation of 4-(1-hydroxy-1,3-dihydrobenzo[c][1,2]oxaborol-6-yl)butanoic acid

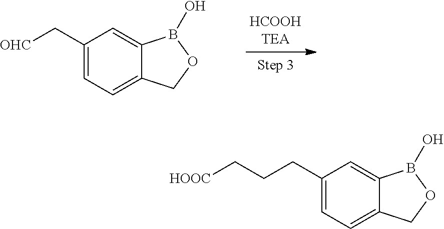

A mixture of 2-(1-hydroxy-1,3-dihydrobenzo[c][1,2]oxaborol-6-yl)acetaldehyde (138 mg, 0.789 mmol, 1.0 eq) and 2,2-dimethyl-1,3-dioxane-4,6-dione (136 mg, 0.941 mmol, 1.2 eq) in (HCOOH/TEA=5:2/volume) (1.0 mL) was heated at 110° C. overnight. TLC showed no starting material remained. The mixture was powered into ice-water (10 mL) and adjusted pH=10 with 1N NaOH. The solution was extracted with ethyl acetate twice. The aqueous phase was adjusted pH=2 with 1N HCl, extracted with ethyl acetate for three times. The organic layers were combined, washed with brine, dried over anhydrous Na₂SO₄, filtered and concentrated. The crude product was purified by preparative TLC plate and then crystallization to give the title compound 4-(1-hydroxy-1,3-dihydrobenzo[c][1,2]oxaborol-6-yl)butanoic acid (12.6 mg, 7.3%). ¹H NMR (300 MHz, DMSO-d₆): δ 12.06 (broad s, 1H), 9.09 (s, 1H), 7.54 (s, 1H), 7.33 (d, J=4.2 Hz, 2H), 4.94 (s, 2H), 2.63 (t, J=7.5 Hz, 2H), 2.21 (t, J=7.3 Hz, 2H), 1.82 (m, 2H). Mass: m/z=255 (M+Na, ESI+) and m/z=219 (M−1, ESI−).

### 67 3-(1-Hydroxy-1,3-dihydrobenzo[c][1,2]oxaborol-6-yl)-N-(cyclopropylsulfonyl) propanamide

The title compound may be prepared by the following scheme.

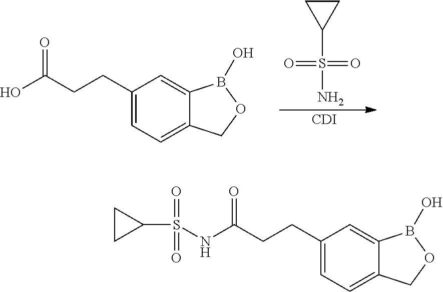

### 68 6-(2-(1H-Tetrazol-5-yl)ethyl)benzo[c][1,2]oxaborol-1(3H)-ol

The title compound may be prepared by the following scheme.

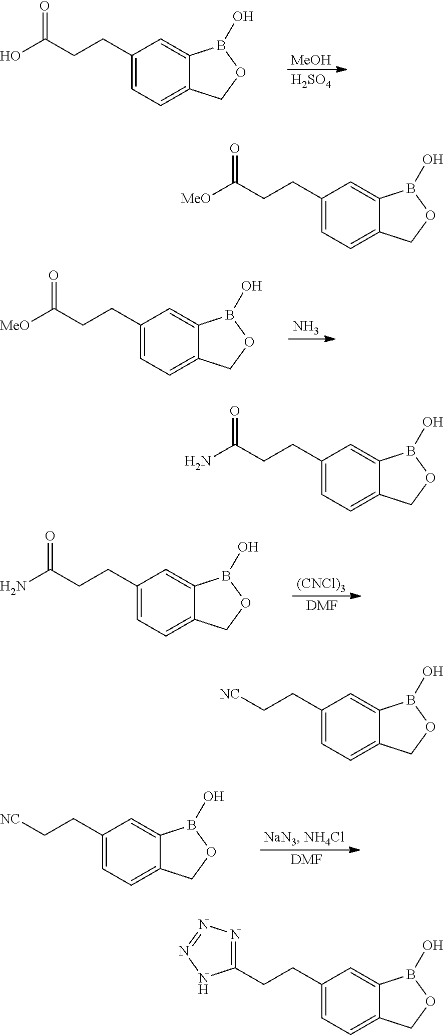

### 69 5-(2-(1-Hydroxy-1,3-dihydrobenzo[c][1,2]oxaborol-6-yl)ethyl)thiazolidine-2,4-dione

The title compound may be prepared by the following scheme.

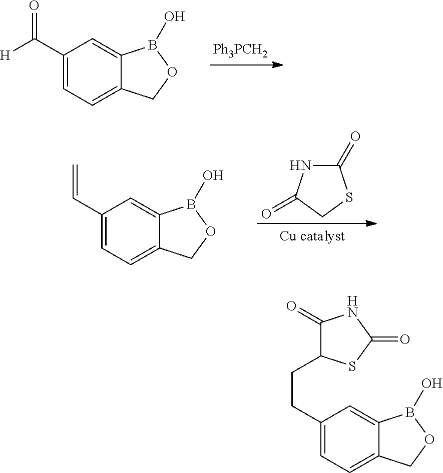

### 70 3-(1-Hydroxy-1,3-dihydrobenzo[c][1,2]oxaborol-5-yl)propanoic acid

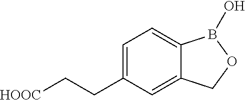

### Step 1: Preparation of 4-bromo-3-(bromomethyl)benzonitrile

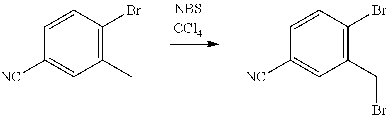

To a solution of 4-bromo-3-methylbenzonitrile (10.0 g, 51.2 mmol, 1 eq) in CCl₄ (128 mL) was added NBS (9.1 g, 51.2 mmol, 1.0 eq), followed by addition of Bz₂O₂ (0.07 g, 0.31 mmol, 0.6% eq). The reaction was heated to reflux under nitrogen overnight. The reaction mixture was cooled to room temperature and filtered. The filtrate was concentrated. The residue was dissolved with ethyl acetate (100 mL), washed with 0.5 HCl (2×50 mL), brine (50 mL) and dried over anhydrous sodium sulfate. The solvent was removed under reduced pressure to give the desired product 4-bromo-3-(bromomethyl)benzonitrile (13.9 g, yield 100%, ˜70% purity). TLC analysis (silica gel plate, EA:PE=20%): R_{f}=0.4.

### Step 2: Preparation of 2-bromo-5-cyanobenzyl acetate

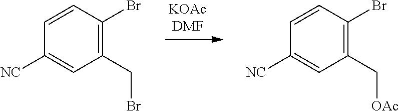

To a solution of 4-bromo-3-(bromomethyl)benzonitrile (53.7 g, 196.8 mmol, 1 eq) in DMF (458 mL) was added KOAc (23.1 g, 236.1 mmol, 1.2 eq). The reaction mixture was stirred at 80° C. for 1.5 h. After being cooled to room temperature, water (1.5 L) was added. The mixture was extracted with ethyl acetate (1 L), washed with 0.5N HCl (3×200 mL), 2% NaHCO₃ (200 mL) and dried over anhydrous sodium sulfate. The solvent was removed. The residue was purified by column chromatography to give desired product 2-bromo-5-cyanobenzyl acetate as a solid (29.6 g, yield 59.5%). TLC analysis (silica gel plate, EA:PE=10%): R_{f}=0.3.

### Step 3: Preparation of 5-cyano-2-(4,4,5,5-tetramethyl-1,3,2-dioxaborolan-2-yl)benzyl acetate

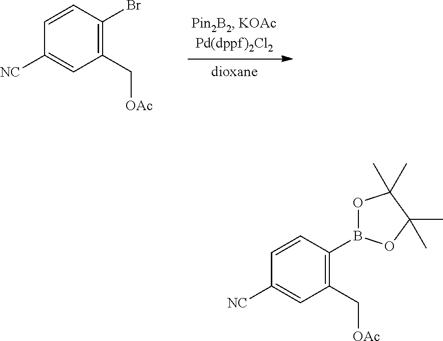

To 2-bromo-5-cyanobenzyl acetate (11 g, 43.3 mmol, 1 eq) in 1,4-dioxane (216.5 mL) was added bis(pinacolato)diboron (16.5 g, 64.9 mmol, 1.5 eq), KOAc (18.2 g, 186.2 mmol, 4.3 eq). The reaction flask was vacuumed and backfilled with nitrogen for 15 min. Pd(dppf)₂Cl₂ (0.8 g, 1.08 mmol, 0.025 eq) was added. The reaction was stirred under nitrogen at reflux overnight. The reaction mixture was cooled and filtered. The filtrate was concentrated. The residue was purified by column chromatography to give desired product 5-cyano-2-(4,4,5,5-tetramethyl-1,3,2-dioxaborolan-2-yl)benzyl acetate (8.0 g, yield 61.5%, purity ˜70%). TLC analysis (silica gel plate, EA:PE=10%): R_{f}=0.3.

### Step 4: Preparation of 1-hydroxy-1,3-dihydrobenzo[c][1,2]oxaborole-5-carbonitrile

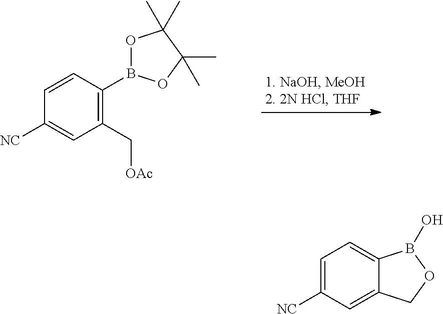

To 5-cyano-2-(4,4,5,5-tetramethyl-1,3,2-dioxaborolan-2-yl)benzyl acetate (8.0 g, 26.5 mmol) in methanol (40.4 mL) was added a solution of NaOH in MeOH (2.4 g in 26 mL, 61.1 mmol, 2.3 eq). The reaction was stirred at room temperature for 2 h. The reaction mixture was concentrated. The residue was dissolved in THF (40 mL) and 2N HCl (11.9 mL, 23.9 mmol, 0.9 eq). The reaction was stirred at room temperature for 50 min. The reaction mixture was concentrated. The residue was purified by column chromatography to give desired product 1-hydroxy-1,3-dihydrobenzo[c][1,2]oxaborole-5-carbonitrile (1.94 g, yield 46.2%). TLC analysis (silica gel plate, EA: PE=25%): R_{f}=0.2.

### Step 5: Preparation of 1-hydroxy-1,3-dihydrobenzo[c][1,2]oxaborole-5-carbaldehyde

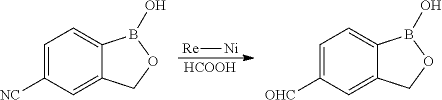

To a mixture of Raney Ni (424 mg, 7.2 mmol, 2.3 eq), formic acid (5 mL) and water (1 mL) was added 1-hydroxy-1,3-dihydrobenzo[c][1,2]oxaborole-5-carbonitrile (500 mg, 3.14 mmol, 1 eq). The reaction was stirred at 100° C. for 1.5 h. The mixture was filtered. The solvent was removed. The residue was purified by column chromatography to give desired product 1-hydroxy-1,3-dihydrobenzo[c][1,2]oxaborole-5-carbaldehyde as a solid (0.310 mg, yield 60.8%). TLC analysis (silica gel plate, EA:PE=25%): R_{f}=0.4. ¹H NMR (300 MHz, DMSO-d₆): δ 10.09 (s, 1H), 9.45 (s, 1H), 7.92 (m, J=10.7 Hz, 3H), 5.09 (s, 2H) ppm.

### Step 6: Preparation of 3-(1-hydroxy-1,3-dihydrobenzo[c][1,2]oxaborol-5-yl)propanoic acid

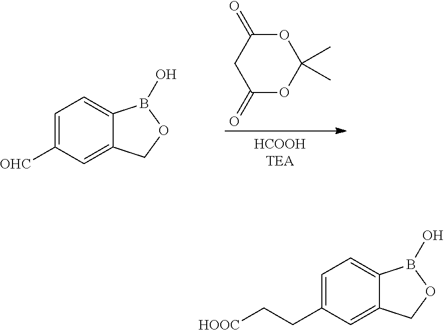

A mixture of 1-hydroxy-1,3-dihydrobenzo[c][1,2]oxaborole-5-carbaldehyde (200 mg, 1.23 mmol, 1.0 eq) and 2,2-dimethyl-1,3-dioxane-4,6-dione (196.5 mg, 1.35 mmol, 1.1 eq) in (HCOOH/TEA=5:2/volume) (1.2 mL) was heated at 110° C. for 2 h. TLC showed no starting material remained. The mixture was powered into ice-water (10 mL) and adjusted pH=10 with 1N NaOH. The solution was extracted with ethyl acetate twice. The aqueous phase was adjusted pH=2 with 1N HCl, extracted with ethyl acetate for three times. The organic layers were combined, washed with brine, dried over anhydrous Na₂SO₄, filtered and concentrated. The crude product was purified by preparative TLC plate to give 3-(1-hydroxy-1,3-dihydrobenzo[c][1,2]oxaborol-5-yl)propanoic acid (37 mg, yield 14.5%) as a white solid. ¹H NMR (300 MHz, DMSO-d₆): δ 12.13 (s, 1H), 9.10 (s, 1H), 7.64 (d, J=3 Hz, 1H), 7.22 (m, J=9 Hz, 2H), 4.97 (s, 2H), 2.87 (t, J=9 Hz, 2H), 2.58 (m, 2H) ppm. Mass: m/z=205.3 (M−1, ESI−).

### 71 3-(1-Hydroxy-1,3-dihydrobenzo[c][1,2]oxaborol-4-yl)propanoic acid

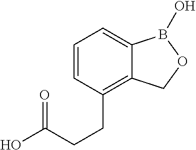

The title compound was prepared with a method similar to that described in 3-(1-hydroxy-1,3-dihydrobenzo[c][1,2]oxaborol-5-yl)propanoic acid by starting with 3-bromo-2-methylbenzonitrile. ¹H NMR (300 MHz, DMSO-d₆): δ 12.10 (s, 1H), 9.08 (s, 1H), 7.57 (d, J=2.5 Hz, 1H), 7.29 (m, J=1.8 Hz, 2H), 5.03 (s, 2H), 2.76 (t, J=2.3 Hz, 2H), 2.53 (m, 2H) ppm. Mass: m/z=205.3 (M−1, ESI−). Purity: 96.72% at 220 nm.

### 72 3-(1-Hydroxy-1,3-dihydrobenzo[c][1,2]oxaborol-6-yl)benzoic acid

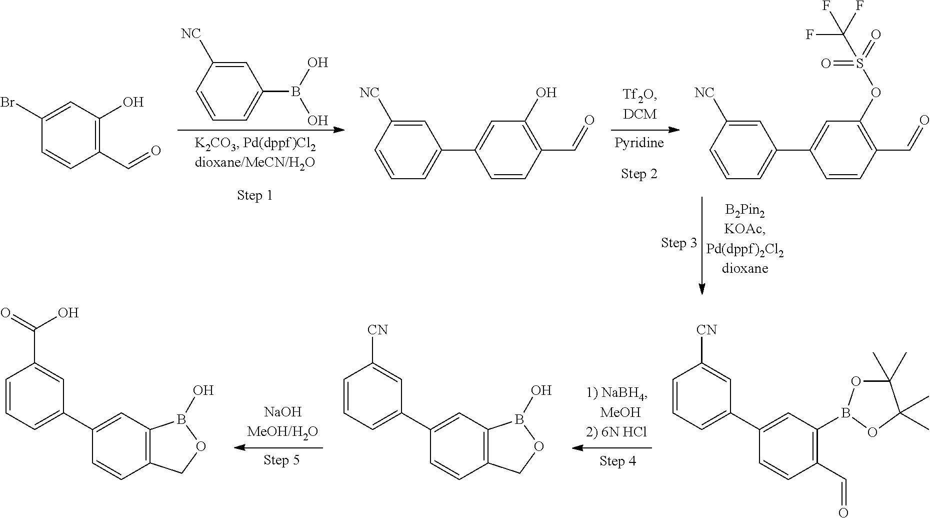

### Step 1: Preparation of 4′-formyl-3′-hydroxybiphenyl-3-carbonitrile

To a solution of 4-bromo-2-hydroxybenzaldehyde (4 g, 20 mmol) in dioxane/MeCN/H₂O (72 ml/24 ml/24 ml) was added 3-cyanophenylboronic acid (3.52 g, 24 mmol), K₂CO₃ (4.14 g, 30 mmol) and Pd(dppf)₂Cl₂ (0.74 g, 1 mmol). The solution was stirred at 80° C. under N₂ overnight. The mixture was filtered through celite and the filtrate was concentrated to give the crude product that was purified by silica-gel column chromatography (PE:EA 15:15:1) to give the coupling product 4′-formyl-3′-hydroxybiphenyl-3-carbonitrile (3.8 g, yield 85%) as a yellow solid. ¹H NMR (400 MHz, CDCl₃): δ 11.12 (s, 1H), 9.95 (s, 1H), 7.89 (s, 1H), 7.83-7.89 (m, 1H), 7.66-7.72 (m, 2H), 7.59 (t, J=8 Hz, 1H), 7.18-7.23 (m, 2H) ppm.

### Step 2: Preparation of 3′-cyano-4-formylbiphenyl-3-yl trifluoromethanesulfonate

To a solution of 4′-formyl-3′-hydroxybiphenyl-3-carbonitrile (3.189 g, 14.3 mmol) in dry DCM (500 ml) was added pyridine (2.32 ml, 28.6 mmol) at 0° C. and stirred for 0.5 hour. Then Tf₂O (3.62 ml, 21.45 mmol) was added at 0° C. to the reaction mixture. After stirring at 0° C. for 0.5 hour, the solution was stirred at room temperature for 1 hour. The mixture was washed with water and brine. The DCM layer was dried over Na₂SO₄ and concentrated to give 3′-cyano-4-formylbiphenyl-3-yl trifluoromethanesulfonate (5.0 g, 98.48%) as a yellow solid. ¹H NMR (400 MHz, CDCl₃): δ 10.32 (s, 1H), 8.12 (d, J=8.0 Hz, 1H), 7.89 (s, 1H), 7.84 (d, J=10.8 Hz, 1H), 7.79-7.74 (m, 2H), 7.68-7.64 (m, 1H), 7.58 (s, 1H) ppm.

### Step 3: Preparation of 4′-formyl-3′-(4,4,5,5-tetramethyl-1,3,2-dioxaborolan-2-yl)biphenyl-3-carbonitrile

To a solution of 3′-cyano-4-formylbiphenyl-3-yl trifluoromethanesulfonate (2.38 g, 6.7 mmol) in dioxane (80 ml) was added Pin₂B₂ (1.87 g, 7.37 mmol), KOAc (1 g, 10 mmol) and Pd(dppf)₂Cl₂ (245 mg, 034 mmol) under N₂. The solution was stirred at 80° C. under N₂ overnight. The mixture was purified by silica-gel column chromatography to give 4′-formyl-3′-(4,4,5,5-tetramethyl-1,3,2-dioxaborolan-2-yl)biphenyl-3-carbonitrile (1.3 g, 58%) as a yellow solid. ¹H NMR (400 MHz, CDCl₃): δ 10.62 (s, 1H), 8.05-8.09 (m, 2H), 7.94 (s, 1H), 7.85-7.87 (m, 1H), 7.75˜7.77 (m, 1H), 7.67˜7.69 (m, 1H), 7.61 (t, 1H, J=8 Hz), 1.42 (s, 12H) ppm.

### Step 4: Preparation of 3-(1-hydroxy-1,3-dihydrobenzo[c][1,2]oxaborol-6-yl)benzonitrile

To a stirring suspension of 4′-formyl-3′-(4,4,5,5-tetramethyl-1,3,2-dioxaborolan-2-yl)biphenyl-3-carbonitrile (333 mg, 1 mmol) in dry MeOH (7 ml) was added NaBH₄ (270 mg, 7.1 mmol) in portions at 0° C. The solution was stirred at 0° C. for 0.5 h. Then the solution was concentration under reduced pressure, and 6N HCl (6 ml) was added to the mixture. And it was stirred at room temperature for 2 h. The mixture was concentrated in vacuum and the residue washed with MeOH to give 3-(1-hydroxy-1,3-dihydrobenzo[c][1,2]oxaborol-6-yl)benzonitrile (200 mg, yield 85%) as a white solid. ¹H NMR (400 MHZ, DMSO-d₆): δ 9.40 (s, 1H), 8.12 (d, 1H, J=8.0 Hz), 8.00 (d, 1H, J=8.0 Hz), 7.82-7.84 (m, 3H), 7.69 (t, J=8. Hz, 1H), 7.53 (d, J=8. Hz, 1H), 5.04 (s, 2H) ppm; MS: m/z 236 (M+1, ESI+); HPLC purity: 99.65% at 220 nm.

### Step 5: Preparation of 3-(1-hydroxy-1,3-dihydrobenzo[c][1,2]oxaborol-6-yl)benzoic acid

To a solution of 3-(1-hydroxy-1,3-dihydrobenzo[c][1,2]oxaborol-6-yl)benzonitrile (306 mg, 1.3 mmol) in MeOH/H₂O (7 ml/7 ml) was added NaOH (520 mg, 13 mmol). The solution was stirred at 80° C. for overnight. MeOH was evaporated in vacuum. The resulting aqueous layer was extracted with t-butyl methyl ether and the aqueous solution was acidified to pH 1 with 2N HCl to give the desired product 3-(1-hydroxy-1,3-dihydrobenzo[c][1,2]oxaborol-6-yl)benzoic acid as a white solid (210 mg, yield 63.6%) as a white solid. ¹H NMR (400 MHz, DMSO-d₆): δ 9.40 (s, 1H), 8.21 (s, 1H), 8.07 (s, 1H), 7.95-7.91 (m, 2H), 7.81 (d, J=9.6 Hz, 1H), 7.63-7.59 (m, 1H), 7.54-7.52 (m, 1H) and 5.04 (s, 2H) ppm; MS: m/z 255 (M+1, ESI+); HPLC purity: 96.36% at 220 nm.

### Example 2

### Trypanosoma brucei brucei High-Throughput Screening Assay Procedure

All experiments were conducted with the bloodstream-form trypanosome T. brucei brucei 427 strain obtained from Seattle Biomedical Research Institute (Seattle, Wash.). Parasites were cultured in T-25 vented cap flasks and kept in humidified incubators at 37° C. and 5% CO₂. The parasite culture media was complete HMI-9 medium (c.f. Hirumi, Journal of Parasitology 1989, Volume 75, page 985 et seq) containing 10% FBS, 10% Serum Plus medium and penicillin/streptomycin. To ensure log growth phase, trypanosomes were sub-cultured at appropriate dilutions every 2-3 days.

### In Vitro Drug Sensitivity Assays

Approximately 50 microliters of log phase cultures were diluted 1:10 in HMI-9 and 10 uL of the diluted culture was removed and counted using a hemocytometer to determine parasite concentration. Parasites were diluted by addition of an appropriate volume of HMI-9 to achieve a final parasite concentration of 2×10⁵/mL. Compounds of the invention to be tested were serially diluted in DMSO and 0.5 uL added to 49.5 uL HMI-9 in triplicate 96-well plates using a Biomek NX liquid handler. Parasites from the diluted stock were added to each well (50 uL) using a Multidrop 384 dispenser to give a final concentration of 1.0×105/ml parasites in 0.4% for DMSO. Trypanosomes were incubated with compounds for 72 hrs at 37° C. with 5% CO₂. Resazurin (20 uL of 12.5 mg/ml stock) from Sigma-Aldrich was added to each well and plates were incubated for an additional 2-4 hrs. Assay plates were read using an EnVision plate reader at an excitation wavelength of 544 nm and emission of 590 nm. Triplicate data points were averaged to generate sigmoidal dose response curve and determine IC₅₀ values using XLfit curve fitting software from IDBS (Guildford, UK).

Biological data for exemplary compounds of the invention is provided in FIG. 1.

### Example 3

### Activity Against Plasmodium falciparum

Chloroquine-resistant P. falciparum (W2 strain) parasites were cultured in human erythrocytes in RPMI culture media containing 2% human serum and 0.5% Albumax serum substitute. After a 48 h incubation with test concentrations or serial dilutions of compounds in microtiter plates, cultures were fixed in 1% formaldehyde, incubated with YOYO-1 nuclear stain and evaluated by flow cytometry with gating to separate infected from uninfected cells. Infected erythrocytes/10,000 cells were counted and IC50 values calculated using Prism (GraphPad Software).

Biological data for exemplary compounds of the invention is provided in FIG. 1.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound having a structure according to the following formula: 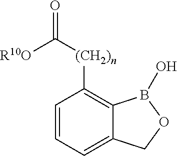
wherein n is 1 or 2 or 3 or 4 or 5, and R¹⁰ is H or C₁-C₆ alkyl, or a salt thereof.

2. The compound of claim 1, having a structure according to the following formula: 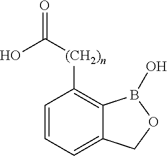
wherein n is 1 or 2 or 3 or 4 or 5.

3. The compound of claim 1, which is

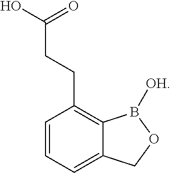

4. A combination comprising the compound of a preceding claim, together with at least one other therapeutically active agent.

5. A pharmaceutical formulation comprising:
a) the compound of a preceding claim, or a salt thereof; and
b) a pharmaceutically acceptable excipient.

6. The pharmaceutical formulation of claim 5, wherein the pharmaceutical formulation is a unit dosage form.

7. The pharmaceutical formulation of claim 5, wherein the salt of said compound of a preceding claim is a pharmaceutically acceptable salt.

8. A method of killing and/or preventing the growth of a protozoa, comprising: contacting the protozoa with an effective amount of the compound of the invention, thereby killing and/or preventing the growth of the protozoa.

9. The method of claim 8, wherein the compound is according to claim 1.

10. The method of claim 8, wherein the protozoa is a member selected from the trypanosoma genus and the plasmodium genus.

11. The method of claim 8, wherein the protozoa is Trypanosoma brucei.

12. The method of claim 11, wherein the Trypanosoma brucei is a member selected from Trypanosoma brucei brucei, Trypanosoma brucei gambiense and Trypanosoma brucei rhodesiense.

13. The method of claim 8, wherein the protozoa is a member selected from Plasmodium falciparum, Plasmodium vivax, Plasmodium ovale, Plasmodium vivax, Plasmodium malariae and Plasmodium knowlesi.

14. The method of claim 13, wherein the protozoa is Plasmodium falciparum.

15. A method of treating and/or preventing a disease in an animal, comprising: administering to the animal a therapeutically effective amount of the compound of the invention, thereby treating and/or preventing the disease.

16. The method of claim 15, wherein the compound is according to claim 1.

17. The method of claim 15, wherein the disease is African sleeping sickness.

18. The method of claim 15, wherein the disease is malaria.

19. The method of claim 15, wherein the animal is a human.

20. A use of the compound of claim 1 in the manufacture of a medicament for the treatment and/or prophylaxis of protozoal infection.
